(19) United States
(12) Patent Application Publication
(10) Pub. No.: US 2017/0218006 A1
(43) Pub. Date: Aug. 3, 2017

(54) Synthesis of Phosphoramidates

(71) Applicant: Sandoz AG, Basel (CH)

(72) Inventors: Thorsten Wilhelm, Kundl/Tirol (AT); Hans-Peter Spitzenstätter, Kundl/Tirol (AT); Olga Schöne, Kundl/Tirol (AT); Frank Richter, Kundl/Tirol (AT); Erwin Schreiner, Kundl/Tirol (AT)

(73) Assignee: Sandoz AG, Basel (CH)

(21) Appl. No.: 15/500,627

(22) PCT Filed: Jul. 31, 2015
(86) PCT No.: PCT/EP2015/067720

§ 371 Date: Jan. 31, 2017

(30) Foreign Application Priority Data
Jul. 31, 2014 (EP) 14179385.1

(51) Int. Cl. C07H 19/06; C07F 9/653; C07F 9/6521; C07F 9/572

(52) U.S. Cl. CPC C07H 19/06; C07F 9/5725; C07F 9/65324; C07F 9/65215

(57) ABSTRACT

A process for the preparation of a compound of formula (I) including all isomers, stereoisomers, enantiomers and diastereomers thereof (I), and salts thereof; the process comprising providing a mixture comprising a compound of formula (II) and a compound of formula (III) subjecting the mixture provided in a) to reaction conditions in the presence of one or more Lewis acids to the mixture provided in a), obtaining a mixture comprising the compound of formula (I).

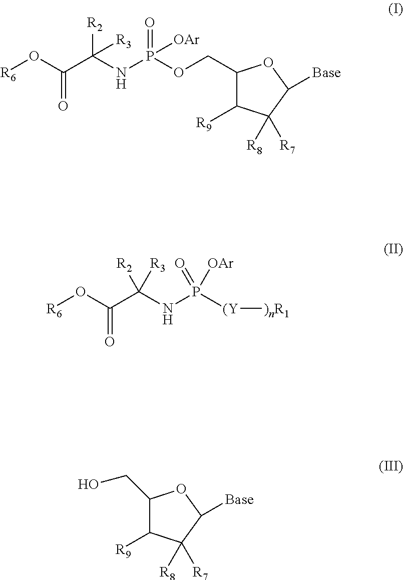

The present invention relates to a novel synthesis for preparing a compound of formula (I)

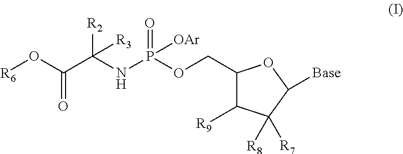

via an intermediate of formula (II)

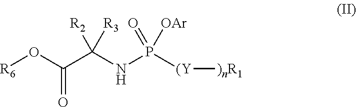

in the presence of a Lewis acid. Further, the present invention relates to the novel intermediate of formula (II) as such.

Sofosbuvir according to the following formula

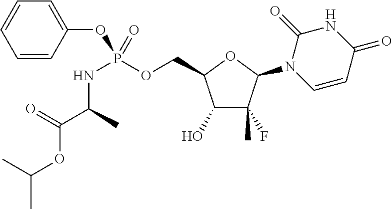 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

with IUPAC name (S)-isopropyl 2-(((S)-(((2R,3R,4R,5R)-5-(2,4-dioxo-3,4-dihydropyrimidin-1 (2H)-yl)-4-fluoro-3-hydroxy-4-methyltetrahydrofuran-2-yl)methoxy) (phenoxy)phosphoryl) amino)propanoate is a drug inhibiting the RNA polymerase used by the hepatitis C virus to replicate its RNA.

Sofosbuvir and similar nucleoside phosphoramidates are generally prepared by displacement of a leaving group (LG) on a phosphoramidate compound by a nucleoside compound. The LG used in the art is a chlorine atom or an aryloxide substituted with at least one electron withdrawing group such as halogen and nitro groups. In particular p-nitrophenol is used as LG.

In WO 2008/121634, a process for preparing nucleoside phosphoramidate compounds is disclosed. The nucleoside phosphoramidates are prepared via displacement of Cl (as LG) on the phosphoramidate compounds by an OH group of a nucleoside/ribose to give the corresponding nucleoside phosphoramidates. The phosphoramidates are used in large excess (3.4 to 6 eq.) with respect to the nucleoside/ribose. N-methylimidazole is used as the base in the displacement reaction. This displacement reaction is not selective. It has been seen that it leads to the formation of side products as the nucleoside moiety (ribose) may comprise an OH group (secondary alcohol) in position 3′ or 2′ of the ribose ring that competes with the primary OH group in position 5′. Hence for example 3′-O-phosphoramidate and/or 3′,5′-bis-O-phosphoramidate are formed as side-products.

In WO 2011/123668, WO 2010/135569, and WO 2011/123645 a process for preparing nucleoside phosphoramidate compounds is disclosed. The nucleoside phosphoramidate is prepared via displacement of a leaving group (LG) on a phosphoramidate by an OH group of a nucleoside to give the corresponding nucleoside-phosphoramidate. A basic reagent such as a Grignard reagent is used in the displacement reaction. The disclosed LG is an aryloxide substituted with at least one electron-withdrawing group such as a halogen or a nitro group. In Example 15 of WO 2010/135569, in order to overcome the problem of the formation of 3′-O-phosphoramidate or 3′,5′-bis-O-phosphoramidate side products, it is proposed to protect position 3′ of the ribose ring with a levulinic anhydride and subsequently de-protect said position. Alternatively, position 3′ is protected with a tert-butyl-dimethylsilyl group.

In WO 2011/123672 a process for preparing nucleoside phosphoramidate compounds is disclosed. The nucleoside phosphoramidate is prepared via displacement of the leaving group (LG) on a phosphoramidate by an OH group of a nucleoside to give the corresponding nucleoside-phosphoramidate. A basic reagent such as a Grignard reagent is used in the displacement reaction. The disclosed LG is an aryloxide substituted with at least one electron-withdrawing group such as halogen or a nitro group. Alternatively, the LG is a benzo[d]thiazole-2(3H)-thione.

In WO2014/047117 a process for preparing nucleoside phosphoramidate compounds is disclosed. The process is a two-step process. The first step is the displacement of the leaving group (LG) such as p-nitrophenol on a phosphinoborane derivative or on a thio-phosphoramidate compound by an OH group of a nucleoside to give the corresponding nucleoside boran- or thio-phosphoramidate. The displacement occurs in basic conditions (Et₃N, DBU (1,8-Diazabicyclo[5.4.0]undec-7-ene)). In a subsequent step, the nucleoside boran- or thio-phosphoramidate is oxidized to the corresponding nucleoside phosphoramidate.

The nucleoside phosphoramidate prepared according to the above procedures, due to the chirality of the phosphorous atom, comprises two diastereoisomers. For example with reference to sofosbuvir the two diastereoisomers have formulas (SP-I) and (SP-II):

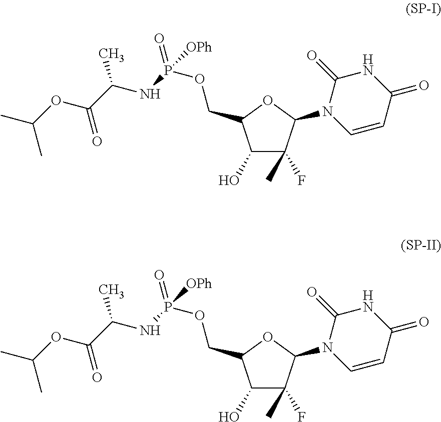

The above mentioned WO 2010/135569 discloses a process for preparing sofosbuvir wherein a diastereoisomeric mixture is obtained having a ratio of SP-I relative to SP-II of about 3:1. In view of this disclosure, it is desirable to provide a synthesis process leading to an improved diastereoselectivity.

An additional problem of the above synthesis relates to the LG used. P-nitrophenol and in general LGs may remain as trace impurity in the final nucleoside phosphoramidate compounds. P-nitrophenol and in general aryloxide substituted with an electron-withdrawing group are considered to be toxic substances, in particular genotoxic substances, by FDA. FDA requires for example a content of less than 20 ppm of these substances to be present in goods. Difficulties may be encountered to purify the final API from these toxic leaving groups to meet the FDA requirements.

Therefore, the problem underlying the present invention is the provision of a novel process for the preparation of nucleoside phosphoramidates that is selective, is carried out in mild and simple conditions, is economic and provides nucleoside phosphoramidates in good yields and diastereoselectivity. Additionally, the process may be carried out with non-toxic reagents.

Surprisingly, it was found that this problem can be solved by a process for preparing nucleoside phosphoramidates which is carried out under acidic conditions provided by a Lewis acid.

Therefore, the present invention relates to a process for the preparation of a compound of formula (I) including all isomers, stereoisomers, enantiomers and diastereomers thereof

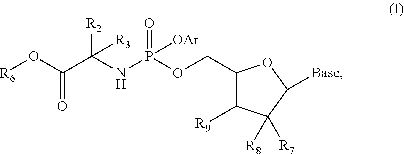

and salts thereof, the process comprising
a) providing a mixture comprising a compound of formula (II)

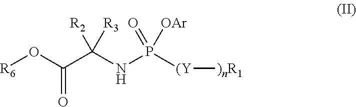

and a compound of formula (III)

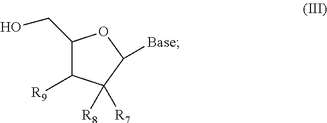

b) subjecting the mixture provided in a) to reaction conditions in the presence of one or more Lewis acids to the mixture, obtaining a mixture comprising the compound of formula (I).

Preferably, the present invention relates to said process wherein at each occurrence

Ar is phenyl, naphthyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
(Y—)ₙR₁ is a leaving group for nucleophilic substitution reaction, wherein n is 0 or 1 and wherein Y is O, N or S;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, COOH, CHO, C(O)(C₁-C₆ alkyl), C(O)(aryl), COO(C₁-C₆ alkyl), COONH₂, COONH(C₁-C₆ alkyl) and CN; R₆ is C₁-C₆ alkyl or C₃-C₁₀ cycloalkyl optionally substituted with at least one of C₁-C₆ alkyl and aryl;
Base is a purinyl residue or a pyrimidinyl residue linked to the furanose ring according to formula (III) through a carbon or nitrogen atom;
R₇ and R₈ are independently H, OH, F, Cl, Br, I, azide, nitrile, NH₂, NHR₂₆, NR₂₆R₂₄, (CO)—NH₂, (CO)—NHR₂₆, (CO)—NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl;
R₉ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl.

Further, the present invention relates to a mixture comprising a compound of formula (I) including all isomers, stereoisomers, enantiomers and diastereomers thereof

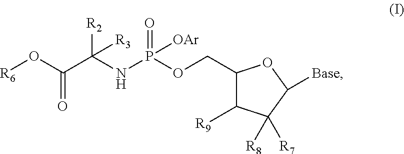

and salts thereof, obtainable or obtained by the process defined above.

Further, the present invention relates a composition of which at least 99.90 weight-%, preferably at least 99.92 weight-%, based on the weight of the composition, consist of the compound of formula (I) including isomers, stereoisomers, enantiomers, diastereomers thereof

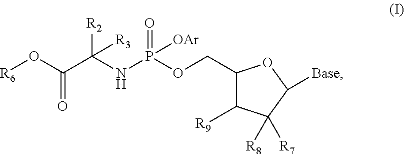

and salts thereof, preferably consist of the compound of formula (I-A)

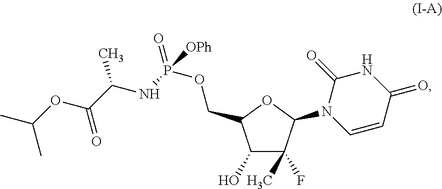

wherein said composition has a content, based on the total weight of the composition, of less than 100 weight-ppm, preferably less than 50 weight-ppm, more preferably less than 10 weight-ppm of an aryl-OH compound substituted with one or more electron-withdrawing groups wherein the one or more electron-withdrawing groups are preferably selected from the group consisting of F, Cl, Br, I, NO₂, CF₃ and a combination thereof, wherein the aryl-OH compound is preferably selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol.

Further, the present invention relates to a process comprising
(i) providing a mixture comprising a compound of formula (IV)

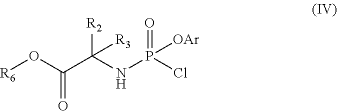

and a compound R₁(—Y)ₙH;
(ii) subjecting the mixture provided in (i) to reaction conditions, obtaining a mixture comprising the compound of formula (II)

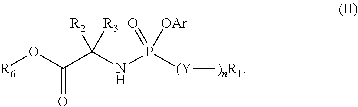

Preferably, the present invention relates to said process wherein at each occurrence

Ar is phenyl, naphthyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
(Y—)ₙR₁ is a leaving group for nucleophilic substitution reaction, wherein n is 0 or 1 and wherein Y is O, N or S;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, COOH, CHO, C(O)(C₁-C₆ alkyl), C(O)(aryl), COO(C₁-C₆ alkyl), COONH₂, COONH(C₁-C₆ alkyl) and CN; R₆ is C₁-C₆ alkyl or C₃-C₁₀ cycloalkyl optionally substituted with at least one of C₁-C₆ alkyl and aryl.

Further, the present invention relates to a mixture comprising a compound of formula (II)

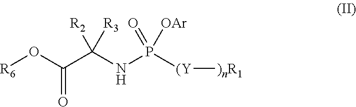

obtainable or obtained by the process defined above.

Further, the present invention relates to a composition of which at least 99.90 weight-%, preferably at least 99.92 weight-% consists of the compound of formula (II)

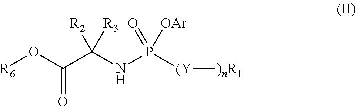

wherein said composition has a content, based on the weight of the mixture, of less than 100 weight-ppm, preferably less than 50 weight-ppm, more preferably less than 10 weight-ppm of an aryl-OH compound substituted with one or more electron-withdrawing groups wherein the one or more electron-withdrawing groups are preferably selected from the group consisting of F, Cl, Br, I, NO₂, CF₃ and a combination thereof, wherein the aryl-OH compound is preferably selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol.

Further, the present invention relates to a compound of formula (II)

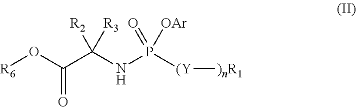

wherein
Ar is phenyl, naphthyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl) and CN; R₆ is C₁-C₆ alkyl or C₃-C₁₀ cycloalkyl optionally substituted with at least one of C₁-C₆ alkyl and aryl;
(Y—)ₙR₁ is a leaving group for nucleophilic substitution reaction, wherein n is 0 or 1 and wherein Y is O, N or S;
wherein, when n is 1,
R₁ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups, more preferably phenyl substituted with one or more electron-withdrawing groups, wherein the one or more electron-withdrawing groups are preferably F, Cl, Br, I, or NO₂; or
R₁ is a residue of formula (A)

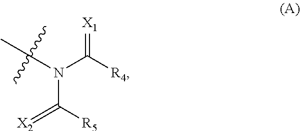

a residue of formula (B)

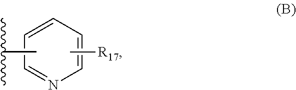

a residue of formula (C)

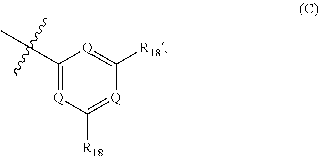

or a residue of formula (D)

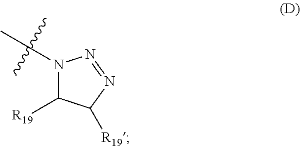

or wherein, when n is 0,
R₁ is a residue of formula (A1)

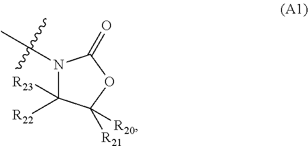

wherein at each occurrence
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.

### Definitions

In the context of the present invention, the term “C₁-C₆ alkyl” refers to a straight or branched saturated monovalent acyclic hydrocarbon radical having 1 carbon atom (C₁), two carbon atoms (C₂), three carbon atoms (C₃), four carbon atom (C₄), five carbon atoms (C₅), or six carbon atoms (C₆). By way of non-limiting examples, C₁-C₆ alkyl includes methyl, ethyl, n-propyl, n-butyl, n-pentyl, n-hexyl, isopropyl, isobutyl, sec-butyl, tert-butyl.

In the context of the present invention, the term “C₁-C₆ alkoxy” refers to the group alkyl-O—, where alkyl is C₁-C₆ alkyl as defined above. By way of non-limiting examples, C₁-C₆ alkyl includes methoxy, ethoxy, n-propoxy, n-butoxy, n-pentoxy, n-hexoxy, isopropoxy, isobu-toxy, sec-butoxy, tert-butoxy.

In the context of the present invention, the term “halogen” refers to halogen atoms such as I, Br, CI and F.

In the context of the present invention, the term “aryl” refers to a monovalent unsaturated aromatic carbocyclic radical having one, two, three, four, five or six rings, preferably one, two or three rings, which may be fused or bicyclic. Preferred aryl groups include an aromatic monocyclic ring containing 6 carbon atoms, an aromatic bicyclic or fused ring system containing 7, 8, 9, or 10 carbon atoms; an aromatic tricyclic ring system containing 10, 11, 12, 13 or 14 carbon atoms. Non-limiting examples of aryl include phenyl and naphthyl. These compounds may include substituent groups, preferably those substituent groups independently selected from hydroxy (—OH), acyl (R′—C(═O)), acyloxy (R′—C(O)—O—), nitro (—NO₂), amino (—NH₂), carboxyl (—COOH), cyano (—CN), thiol (—SH), —Cl, —Br, F—, —I, —SO₃H, —SH, —SR′, wherein R′ includes halogen, C₁-C₆ alkoxy, C₁-C₆ alkyl.

In the context of the present invention, the term “heterocycle” or “heterocyclic” refers to an unsubstituted or substituted heterocycle and further refers to any stable monocyclic, bicyclic, or tricyclic ring which is saturated, unsaturated, or aromatic, and comprises carbon atoms and one or more ring heteroatoms, e. g., 1 or 1 to 2, or 1 to 3, or 1 to 4, or 1 to 5, or 1 to 6 heteroatoms including N, O, S. A bicyclic or tricyclic heterocycle may have one or more heteroatoms located in one ring, or the heteroatoms may be located in more than one ring. The nitrogen and sulfur heteroatoms may optionally be oxidized (i. e., N(O) and S(O)ₚ, where p=1 or 2). When a nitrogen atom is included in the ring, it is either N or NH, depending on whether or not it is attached to a double bond in the ring (i. e., a hydrogen is present if needed to maintain the tri valency of the nitrogen atom). The nitrogen atom may be substituted or unsubstituted (i. e., N or NR wherein R is H or another suitable substituent). The heterocyclic ring may be attached to its pendant group at any heteroatom or carbon atom that results in a stable structure. The heterocyclic rings described herein may be substituted on carbon or on a nitrogen atom. A nitrogen in the heterocycle may optionally be quaternized.

In the context of the present invention, substituents according to the present invention, unless otherwise specified, are substituents selected from the group consisting of OH, C₁-C₆ alkyl, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, wherein aryl is preferably phenyl, heteroaryl. The substituent when present is at least one substituent, preferably one substituent. Hence the term “optionally substituted” means that a chemical group optionally bears at least one of the above mentioned substituents.

In the context of the present invention, the term “purine or pyrimidine base” refers to a nucleoside base such as adenine, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, 5-azacytidinyl, 5-azauracilyl, triazolopyridinyl, imidazolopyridinyl, pyrrolopyrimidinyl, and pyrazolopyrimidinyl. Purine bases include guanine, adenine, hypo-xanthine, 2,6-diaminopurine, and 6-chloropurine. The naturally occurring or modified purine and pyrimidine according to the term “base” in the formulas of the present invention are linked to the ribose sugar through a nitrogen atom or carbon atom of the base. Functional oxygen and nitrogen groups on the base can be optionally protected with suitable protecting groups known to the skilled person in the art, and include trimethylsilyl, dimethylhexylsilyl, t-butyldimethylsilyl, and t-butyldiphenylsilyl, trityl, alkyl groups, and acyl groups such as ace-tyl and propionyl, methanesulfonyl, and p-toluenesulfonyl.

In the context of the present invention, the term “diastereomerically enriched” refers to an instance where, due to the stereochemical information at phosphorus, the mole amount of one diastereomer (Rp or Sp) exceeds the mole amount of the other diastereomer. The phosphorus atoms in the compounds of the present invention are stereogenic. Therefore, the term “diastereomerically enriched” means a composition having from 51 mol-% to 100 mol-% of one diastereomer (with stereochemistry at phosphorous of either Sp or Rp) and from 49 mol-% to 0 mol-% of the other diastereoisomer (Rp or Sp). Within this meaning, the term “diastereomerically enriched” includes a composition comprised of from 60 mol-% of one diastereomer and 40 mol % of the other diastereomer, 70 mol-% of one diastereomer and 30 mol-% of the other diastereomer, 80 mol-% of one diastereomer and 20 mol-% of the other diastereomer, 90 mol-% of one diastereomer and 10 mol-% of the other diastereomer, 95 mol-% of one diastereomer and 5 mol-% of the other diastereomer, 97 mol-% of one diastereomer and 5 mol-% of the other diastereomer, 98 mol-% of one diastereomer and 2 mol-% of the other diastereomer, 99 mol % of one diastereomer and 1 mol-% of the other diastereomer, 99.5 mol-% of one diastereomer and 0.5 mol-% of the other diastereomer, 99.9 mol-% of one diastereomer and 0.1 mol-% of the other diastereomer.

In the context of the present invention, the term “pharmaceutically acceptable salt” refers to a pharmaceutically acceptable salt of a compound, which salt may be derived from a variety of organic and inorganic counter ions known in the art and include, by way of example, sodium, potassium, calcium, magnesium, ammonium, tetraalkylammonium, and when the molecule contains a basic functionality, salts of organic or inorganic acids, such as hydrochloride, hy-drobromide, tartrate, mesylate, acetate, maleate, oxalate and the like.

### Process for Preparing a Compound of Formula (I)

### Step a)

According to a), a mixture is provided which comprises a compound of formula (II)

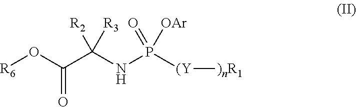

and a compound of formula (III)

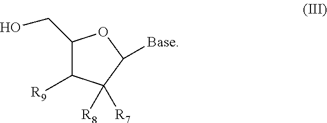

### The Compound of Formula (II)

(Y—)ₙR₁

According to the present invention, the residue (Y—)ₙR₁ is a leaving group which is suitable for a nucleophilic substitution reaction. In the reaction according to b) which will be described in more detail below, the compound of formula (III), in particular the primary alcohol moiety of the compound of formula (III), acts as the nucleophile which substitutes the residue (Y—)ₙR₁ from the compound of formula (II) to form the compound of formula (I) of the invention.

No particular limitation exists with respect to the chemical nature of the group (Y—)ₙR₁ provided that (Y—)ₙR₁ is a suitable leaving group for the above described substitution reaction.

With regard to the leaving group, Y can be present or can be absent. If Y is present, the index n is 1; if Y is absent, the index n is 0. Therefore, the leaving group can be either Y—R₁ or R₁. If Y is present, it is preferably O, N, or S, more preferably O or N, more preferably O. Therefore, if Y is present, preferred leaving groups are O—R₁.

n is 1

If Y is present, no specific limitation exists regarding the chemical nature of R₁ provided that the leaving group is suitable for the above-described reaction.

According to a first preferred alternative, in case n is 1, R₁ is alkyl, preferably C₁-C₆ alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups. Preferably, R₁ is alkyl, preferably C₁-C₆ alkyl, aryl, or heteroaryl, each substituted with one or more electron-withdrawing groups, preferably aryl substituted with one or more electron-withdrawing groups, more preferably phenyl substituted with one or more electron-withdrawing groups. Regarding the chemical nature of the one or more electron-withdrawing groups, no specific limitation exists provided that the leaving group is suitable for the above-described reaction. Preferably, the one or more electron-withdrawing groups are one or more of F, Cl, Br, I, and NO₂. More preferably, the one or more electron-withdrawing groups are one or more of F, Cl, Br and I, more preferably one or more of F and Cl, more preferably F. More preferably, according to the first alternative in case n is 1, R₁ is phenyl substituted with one or more F, and more preferably, the leaving group is O—R₁ wherein R₁ is phenyl substituted with one or more F.

According to a second preferred alternative, in case n is 1, R₁ is a residue of formula (A)

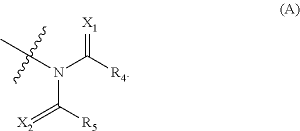

Regarding the chemical nature of X₁, X₂, R₄ and R₅, no specific limitation exists, provided that the leaving group is suitable for the above-described reaction.

Preferably, X₁ and X₂ are independently O or S. More preferably, both X₁ and X₂ are O.

The residues R₄ and R₅ can be either individual residues or can be connected to form a ring structure, preferably to a 5-, 6-, or 7-membered ring structure. This ring structure can be fused, in turn, to at least one further ring, preferably one further ring, preferably a 5- or 6-membered. If R₄ and R₅ are individual residues, R₄ and R₅ are preferably independently H, OH, NH₂, C₁-C₆ alkyl or C₁-C₆ alkoxy. If R₄ and R₅ are connected to form a ring structure, it is preferred that R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S. If R₄ and R₅ are connected to form a ring structure, it is more preferred that R₄ and R₅, together with the structure —C—N—C— according to formula (A), form an optionally substituted, 5-membered saturated ring, wherein said ring is optionally fused to a 5- or 6-membered, optionally substituted ring which is a C₅-C₆ cycloalkyl, an aryl or a heterocycle comprising one or more heteroatoms independently being N, O or S. If R₄ and R₅ are connected to form a ring structure, it is more preferred that R₄ and R₅, together with the structure —C—N—C— according to formula (A), form a 5-membered saturated ring, wherein said ring is optionally fused to a 6-membered, optionally substituted ring which is aryl.

According to a preferred embodiment of the present invention, in case n is 1, R₁ is a residue of formula (IIc)

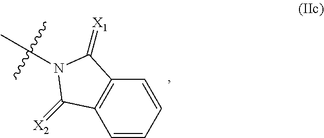

wherein both X₁ and X₂ are preferably O. More preferably, the leaving group is O—R₁ wherein R₁ is the residue of formula (IIc).

According to a preferred embodiment of the present invention, in case n is 1, R₁ is a residue of formula (IIb)

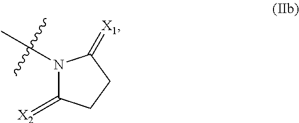

wherein both X₁ and X₂ are preferably O. More preferably, the leaving group is O—R₁ wherein R₁ is the residue of formula (IIb).

According to a third preferred alternative, in case n is 1, R₁ is a residue of formula (B)

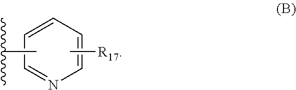

Regarding the chemical nature of R₁₇, no specific limitation exists, provided that the leaving group is suitable for the above-described reaction. Preferably, R₁₇ is an electron-withdrawing group, more preferably R₁₇ is selected from the group consisting of F, Cl, Br, I, NO₂, CHO, COOH, COO—(C₁-C₆)alkyl, CN, and COCl, more preferably F, Cl, NO₂, more preferably NO₂. It is also possible that more than one electron-withdrawing groups R₁₇ can be present in the residue of formula (B), for example 2 or 3 electron-withdrawing groups R₁₇. If more than one electron-withdrawing group R₁₇ are present in the residue of formula (B), they can be of iden-tical or different chemical nature and are preferably from the group described above. If one electron-withdrawing groups R₁₇ are present, which is preferred, R₁₇ is preferably in position meta with respect to the nitrogen atom of the pyridine ring.

Generally, the pyridine residue (B) can be attached to Y in 2-, 3-, or 4-position of the pyridine ring, preferably in 2-position.

More preferably, the leaving group is O—R₁ wherein R₁ is the residue of formula (B).

According to a fourth preferred alternative, in case n is 1, R₁ is a residue of formula (C)

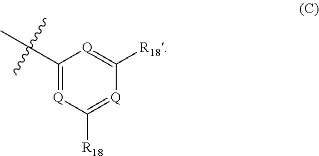

Regarding the chemical nature of Q, R₁₈ and R_{18′}, no specific limitation exists, provided that the leaving group is suitable for the above-described reaction. Preferably, R₁₈ and R_{18′} are independently selected from the group consisting of F, Cl, Br, I, or C₁-C₆ alkoxy, preferably methoxy. Preferably, each Q is independently C or N, wherein at least one Q, such as one Q, two Qs, or three Qs, is N. More preferably, the leaving group is O—R₁ wherein R₁ is the residue of formula (C).

According to a fifth alternative, in case n is 1, R₁ is a residue of formula (D)

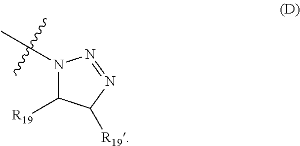

Regarding the chemical nature of Q, R₁₉ and R_{19′}, no specific limitation exists, provided that the leaving group is suitable for the above-described reaction. The residues R₁₉ and R_{19′} can be either individual residues or can be connected to form a ring structure, preferably to a 5-, 6-, or 7-membered ring structure. This ring structure can be fused, in turn, to at least one further ring, preferably one further ring, preferably a 5- or 6-membered.

If R₁₉ and R_{19′} are individual residues, R₁₉ and R_{19′} are preferably independently H, OH, NH₂, C₁-C₆ alkyl optionally substituted with at least one of OH and NH₂, or C₁-C₆ alkoxy optionally substituted with at least one of OH and NH₂.

If R₁₉ and R_{19′} are connected to form a ring structure, it is preferred that R₁₉ and R_{19′}, together with the structure —C—C— according to formula (D), form an optionally substituted, 5-, 6-, or 7-membered saturated or partially unsaturated ring or an aromatic ring, wherein the aromatic ring is preferably benzo, wherein said ring is optionally fused to a 5- or 6-membered, optionally substituted ring which is a C₅-C₆ cycloalkyl, an aryl, preferably benzo, or a heterocycle comprising one or more heteroatoms independently being N, O or S. If R₁₉ and R_{19′} are connected to form a ring structure, it is more preferred that R₁₉ and R_{19′}, together with the structure —C—C— according to formula (D), form an optionally substituted, 5-, 6-, or 7-membered saturated or partially unsaturated ring, wherein said ring is optionally fused to a 5- or 6-membered, optionally substituted ring which is a heterocycle comprising one or more heteroatoms independently being N, O or S.

The substituents of the optionally substituted, 5-, 6-, or 7-membered saturated or partially unsaturated ring or aromatic ring are selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, wherein aryl is preferably phenyl, heteroaryl. The substituent when present is at least one substituent, preferably one substituent.

If R₁₉ and R_{19′} are connected to form a benzo structure, it is preferred that the benzo is substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, wherein aryl is preferably phenyl, heteroaryl.

More preferably, the leaving group is O—R₁ wherein R₁ is the residue selected from the group consisting of a residue of formula (IIb), of formula (IIc) or of formula (D).

n is 0

If Y is not present, no specific limitation exists regarding the chemical nature of R₁ provided that the leaving group is suitable for the above-described reaction.

Preferably, in case n is 0, R₁ is a residue of formula (A1)

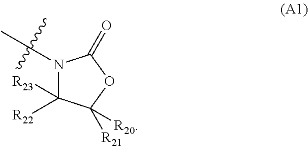

Regarding the chemical nature of R₂₀, R₂₁, R₂₂ and R₂₃, no specific limitation exists, provided that the leaving group is suitable for the above-described reaction. The residues R₂₀, R₂₁, R₂₂ and R₂₃ can be individual residues. Alternatively, two of these residues are individual residues, and two of these residues are connected to form a ring structure.

If these residues are individual residues, R₂₀, R₂₁, R₂₂ and R₂₃ are preferably each independently H, aryl, or C₁-C₆ alkyl optionally substituted with at least one of C₁-C₆ alkoxy optionally substituted with at least one of OH and NH₂, more preferably aryl or alkyl. Preferably, if R₂₂ and R₂₃ are individual residues are each independently H, aryl, or C₁-C₂ or C₄-C₆ alkyl optionally substituted with at least one of C₁-C₆ alkoxy optionally substituted with at least one of OH and NH₂. More preferably, if R₂₂ and R₂₃ are individual residues are each independently H, aryl, or C₁-C₆ alkyl substituted with at least one of C₁-C₆ alkoxy optionally substituted with at least one of OH and NH₂.

If two of these residues are individual residues, and two of these residues are connected to form a ring structure, it is preferred that R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl, and the other two residues, R₂₁ and R₂₃, or R₂₁ and R₂₂, or R₂₀ and R₂₃, or R₂₀ and R₂₂, which do not form the ring structure, are preferably each independently H, aryl, or C₁-C₆ alkyl optionally substituted with at least one of C₁-C₆ alkoxy optionally substituted with at least one of OH and NH₂.

The substituents of the optionally substituted 5-, 6-, or 7-membered saturated or partially unsaturated or aromatic ring which is an aryl, preferably benzo, or a heterocycle comprising one or more heteroatoms independently being N, O or S are selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, wherein aryl is preferably phenyl, heteroaryl. The substituent when present is at least one substituent, preferably one substituent.

If R₂₀ and R₂₂, or R₂₀ and R₂₃, or R₂₁ and R₂₂, or R₂₁ and R₂₃ are connected to form a benzo structure, it is preferred that the benzo is substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, wherein aryl is preferably phenyl, heteroaryl.

According to the present invention, it is also preferred that n is 1 and that the leaving group is Y—R₁ wherein R₁ is a residue selected from the group consisting of a residue of formula (A), a residue of formula (B), a residue of formula (C) and a residue of formula (D) as defined above. It is also preferred that n is 0 and that the leaving group is Y—R₁ wherein R₁ is the residue of formula (A1) as defined above.

According to the present invention, it is also preferred that n is 1 and that the leaving group is Y—R₁ wherein R₁ is the residue of formula (IIc). It is more preferred that n is 1 and that the leaving group is O—R₁ wherein R₁ is the residue of formula (IIc).

According to the present invention, it is more preferred that n is 1 and that the leaving group is Y—R₁ wherein R₁ is the residue of formula (IIb). It is more preferred that n is 1 and that the leaving group is O—R₁ wherein R₁ is the residue of formula (IIb).

Residues R₂ and R₃

Preferably, R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, or carbonyl. More preferably, R₂ and R₃ are independently H and C₁-C₆ alkyl, wherein C₁-C₆ alkyl is preferably methyl. More preferably, one of the residues R₂ and R₃ is H, the other is methyl. More preferably, one of the residues R₂ and R₃ is H and the other is methyl such that the chirality according to the Cahn-Ingold-Prelog (CIP) system of the carbon atom bearing R₂ and R₃ is S.

### Residue R₆

Preferably, R₆ is C₁-C₆ alkyl or C₃-C₁₀ cycloalkyl optionally substituted with at least one of C₁-C₆ alkyl and aryl. More preferably, R₆ is C₁-C₆ alkyl, more preferably methyl, ethyl, isopropyl, or t-butyl, more preferably isopropyl.

### Residue Ar

Preferably, Ar is phenyl, naphthyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN. More preferably, Ar is phenyl or naphthyl, each optionally substituted with at least one C₁-C₆ alkyl, C₁-C₆ alkoxy, C₁-C₆ cycloalkyl, aryl, halogen COOH, CHO, C(O)(C₁-C₆ alkyl), C(O)(aryl), COO(C₁-C₆ alkyl), COONH₂, COONH(C₁-C₆ alkyl) and CN. More preferably, Ar is non-substituted phenyl, non-substituted naphtyl, substituted phenyl or substituted naphthyl wherein in each case the substituent is C₁-C₆ alkyl or C₁-C₆ alkoxy, wherein C₁-C₆ alkyl is preferably methyl and C₁-C₆ alkoxy is preferably methoxy. More preferably, Ar is non-substituted phenyl or non-substituted naphthyl, more preferably non-substituted phenyl.

Therefore, according to an especially preferred embodiment of the present invention, with regard to the compound of formula (II)

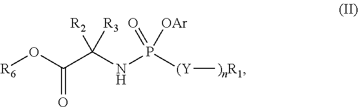

R₆ is isopropyl, one of the residues R₂ and R₃ is H and the other is methyl such that the chirality according to the Cahn-Ingold-Prelog (CIP) system of the carbon atom bearing R₂ and R₃ is S, Ar is phenyl, n is 1, Y is O, and R₁ is preferably a residue selected from the group consisting of a residue of formula (A), a residue of formula (B), a residue of formula (C) and a residue of formula (D) as defined above, more preferably a residue of formula (IIb) or (IIc), even more preferably a residue of formula (IIb)

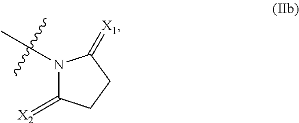

wherein both X₁ and X₂ are O.

According to the present invention, the compound of formula (II) comprises stereogenic atoms, including the phosphorous atom. The preferred isomer is an isomer wherein the stereogenic centers of the compound of formula (II) are as in formula (II′)

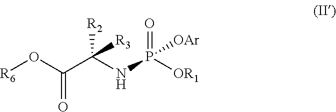

wherein Ar, R₆, R₂, R₃ and R₁ are as defined above, preferably and R₁ is preferably a residue selected from the group consisting of a residue of formula (A), a residue of formula (B), a residue of formula (C) and a residue of formula (D) as defined above, more preferably a residue of formula (IIb) or (IIc), even more preferably a residue of formula (IIb)

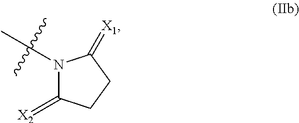

wherein both X₁ and X₂ are O.

According to the present invention, the chirality of each stereogenic center can be assigned according to the Cahn-Ingold-Prelog system (CIP).

According to an even more preferred embodiment of the present with regard to the compound of formula (II), said compound is the compound of formula (II-A)

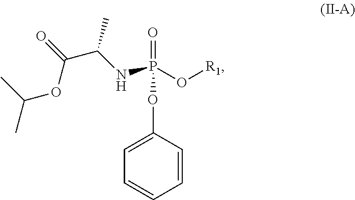

wherein R₁ is the residue selected from the group consisting of a residue of formula (A), a residue of formula (B), a residue of formula (C) and a residue of formula (D) as defined above, more preferably R₁ is the residue of formula (IIb)

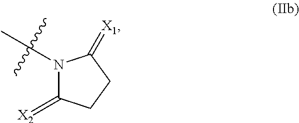

wherein both X₁ and X₂ are O.

Generally, the compound of formula (II-A) can be prepared by any conceivable and suitable process. For example, a possible process for the preparation of the compound of formula (II-A) is disclosed in WO2011/123672, J. Org. Chem 2011, 76, 8311. A process which is preferred according to the present invention is described in detail hereinbelow.

### The Compound of Formula (III)

According to the present invention, the mixture provided in a) contains the compound of formula (III)

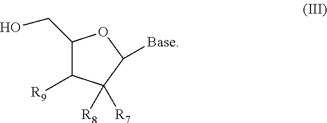

Preferably, R₇ and R₈ are independently H, OH, F, Cl, Br, I, azide, nitrile, NH₂, NHR₂₆, NR₂₆R₂₄, (CO)—NH₂, (CO)—NHR₂₆, (CO)—NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl. More preferably, R₇ and R₈ are independently F, Cl, Br, I or C₁-C₆ alkyl. More preferably, R₇ and R₈ are independently F or methyl. More preferably, one of the two residues R₇ and R₈ is F, the other residue is methyl.

Preferably, R₉ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl. More preferably, R₉ is H or OH, more preferably OH.

Preferably, Base is a purinyl residue or a pyrimidinyl residue linked to the furanose ring according to formula (III) through a C or an N atom, preferably through an N atom.

According to a first preferred embodiment, Base is a residue of formula (B1)

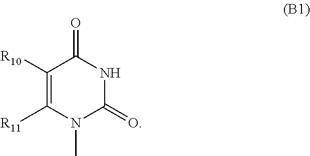

Regarding the residues R₁₀ and R₁₁, no specific limitation exists. Preferably, R₁₀ and R₁₁ are independently H, F, Cl, Br, I, OH, OR, SH, amino, optionally substituted C₁-C₆ alkyl, or optionally substituted C₁-C₆ alkoxy. More preferably, R₁₀ and R₁₁ are independently H or optionally substituted C₁-C₆ alkyl. More preferably, R₁₀ and R₁₁ are H.

According to a second preferred embodiment, Base is a residue of formula (B2)

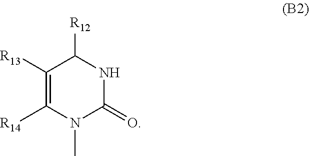

Regarding the residues R₁₂, R₁₃ and R₁₄, no specific limitation exists. Preferably, R₁₂, R₁₃ and R₁₄ are H, F, Cl, Br, I, OH, OR, SH, amino, optionally substituted C₁-C₆ alkyl, or optionally substituted C₁-C₆ alkoxy. More preferably, R₁₂, R₁₃ and R₁₄ are independently H or optionally substituted C₁-C₆ alkyl. More preferably, R₁₂, R₁₃ and R₁₄ are H.

According to a third preferred embodiment, Base is a residue of formula (B3)

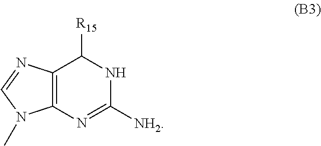

Regarding the residue R₁₅, no specific limitation exists. Preferably, R₁₅, R₁₃ and R₁₄ are H, F, Cl, Br, I, OH, OR, SH, amino, optionally substituted C₁-C₆ alkyl, or optionally substituted C₁-C₆ alkoxy. More preferably, R₁₅ is optionally substituted C₁-C₆ alkoxy. More preferably, R₁₅ is methoxy, ethoxy or iso-propoxy, more preferably methoxy.

According to a fourth preferred embodiment, Base is a residue of formula (B4)

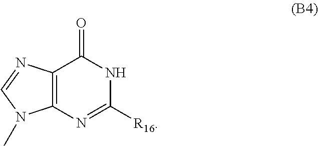

Regarding the residue R₁₆, preferably, R₁₆ is H, F, Cl, Br, I, OH, OR, SH, amino, optionally substituted C₁-C₆ alkyl, or optionally substituted C₁-C₆ alkoxy. More preferably, R₁₆ is H or optionally substituted C₁-C₆ alkyl. More preferably, R₁₆ is H.

According to the present invention, it is preferred that Base is a residue of formula (B1) or a residue of formula (B3). It is more preferred that Base a residue of formula (B1).

Therefore, according to an especially preferred embodiment of the present invention, with regard to the compound of formula (III)

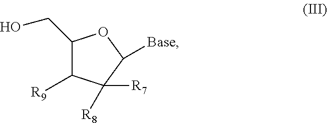

Base a residue of formula (B1), one of the two residues R₇ and R₈ is F, the other residue of R₇ and R₈ is methyl, and R₉ is OH.

According to the present invention, a compound of formula (III) includes all isomers, stereoisomers, enantiomers and diastereomers thereof as the compound of formula (III) comprises at least one stereogenic atom. The preferred isomer is an isomer wherein the stereogenic centers of the compound of formula (III) are as in formula (III′)

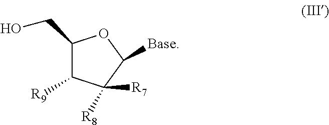

According to the present invention, the chirality of each stereogenic center can be assigned according to the Cahn-Ingold-Prelog system (CIP).

Therefore, according to an especially preferred embodiment of the present invention, with regard to the compound of formula (III), said compound is a compound of formula (III′)

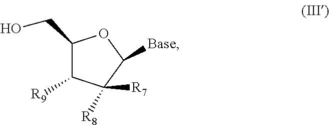

wherein Base a residue of formula (B1), one of the two residues R₇ and R₈ is F, the other residue of R₇ and R₈ is methyl, and R₉ is OH.

According to an even more preferred embodiment of the present with regard to the compound of formula (III), said compound is the compound of formula (III-A)

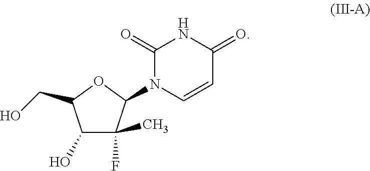

Generally, the compound of formula (III-A) can be prepared by any conceivable and suitable process. For example, a possible process for the preparation of the compound of formula (III-A) is disclosed in J. Med. Chem. 2005, 48, 5504 and J. Org. Chem. 2009, 74, 6819.

Therefore, according to the present invention, it is preferred that the mixture provided in a) contains the compound of formula (II) and the compound of formula (III), wherein the compound of formula (II) is the compound of formula (II-A)

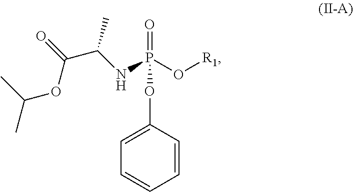

wherein R₁ is the residue of formula (IIb)

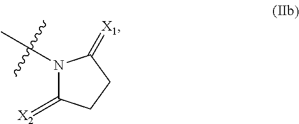

wherein both X₁ and X₂ are O, and the compound of formula (III) is the compound 2′-deoxy-2′fluoro-2′C methyl-uridine of formula (III-A)

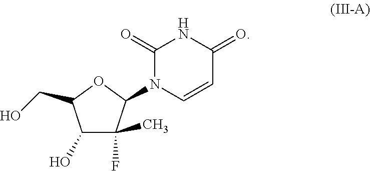

Regarding the molar ratio of the compound of formula (II) relative to the compound of formula (III), no specific limitation exists provided that in b), the compound of formula (I) is obtained. Preferably, in the mixture provided in a), prior to the reaction in b), the molar ratio of the compound of formula (II) relative to the compound of formula (III) is in the range of from 0.5:1 to 5:1, more preferably in the range of from 0.8:1 to 2:1, more preferably in the range of from 0.9:1 to 1.2:1.

According to the present invention, it is preferred that the mixture provided in a) comprises, in addition the compound of formula (II) and the compound of formula (II), one or more solvents. Preferably, the one or more solvents are organic solvents. More preferably, the one or more organic solvents are aprotic organic solvents. More preferably, the one or more solvents are selected from the group consisting of methylene chloride, methyl tert-butyl ether, tetrahydrofurane, dimethylsulphoxide, dimethylformamide, and a mixture of two or more thereof.

According to the present invention, it is preferred that the mixture provided in a) comprises, in addition the compound of formula (II) and the compound of formula (II), and preferably in addition to the one or more solvents, one or more bases. Without wanting to be bound by any theory, it is believed that the one or more bases may at least partially neutralize the acidic compound which is formed during the nucleophilic substitution reaction between the compound of formula (I) and the compound of formula (II). No specific limitation exists with regard to the chemical nature of the one or more bases provided that the reaction according to b) can be carried out, preferably in the one or more solvents mentioned above. Preferably, the one or more bases are organic bases. More preferably, the one or more bases are selected from the group consisting of an amine, an amidine, a heteroaromatic compound comprising a basic ring-nitrogen atom, and a mixture of two or more thereof. More preferably, the one or more bases are selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof.

Therefore, according to the present invention, it is preferred that the mixture provided in a) comprises in addition to the compound of formula (I) and the compound of formula (II), one or more solvents which are preferably selected from the group consisting of methylene chloride, methyl tert-butyl ether, tetrahydrofurane, dimethylsulphoxide, dimethylformamide, and a mixture of two or more thereof, and one or more bases which are preferably selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof.

Regarding the molar ratio of the one or more bases relative to the compound of formula (III), no specific limitation exists provided that in b), the compound of formula (I) is obtained.

Preferably, in the mixture provided in a), prior to the reaction in b), the molar ratio of the one or more bases relative to the compound of formula (III) is in the range of from 0.1:1 to 5:1, more preferably in the range of from 0.1:1 to 2:1 preferably in the range of from 0.5:1 to 1.2:1. If more than one base is comprised in the mixture provided in a), the molar ratio relates to the total molar amount of all bases.

According to the present invention, it is especially preferred that according to a), a mixture is provided which comprises the compound (II), the compound (III), one or more bases and or more solvents, wherein the compound of formula (II) is the compound of formula (II-A)

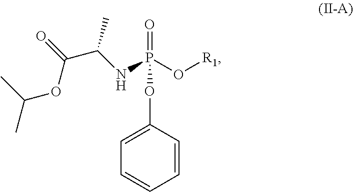

wherein R₁ is the residue of formula (IIb)

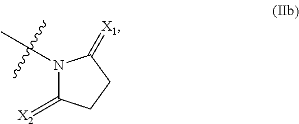

wherein both X₁ and X₂ are 0, the compound of formula (III) is the compound of formula (III-A)

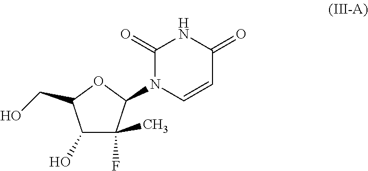

the one or more bases are selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof, the one or more solvents are selected from the group consisting of methylene chloride, methyl tert-butyl ether, tetrahydrofurane, dimethylsulphoxide, dimethylformamide, and a mixture of two or more thereof, wherein prior to the reaction according to b), the molar ratio of the compound of formula (II) relative to the compound of formula (III) is in the range of from 0.9:1 to 1.2:1, and the molar ratio of the one or more bases relative to the compound of formula (III) is in the range of from 0.5:1 to 1.2:1 wherein, if more than one base is comprised in the mixture provided in a), the molar ratio relates to the total molar amount of all bases.

### Step b)

According to the present invention, the reaction in b) takes place in the presence of one or more Lewis acids. Therefore, the mixture which is subjected to reaction in b) further comprises one or more Lewis acids. The sequence of mixing the compounds of formulas (II) and (III), preferably the one or more solvents, preferably the one or more bases, and the one or more Lewis acids is not specifically critical.

Preferably, the reaction in b) is carried out in the presence of a suitable adsorbent. Therefore, in addition to the compound of formula (II), the compound of formula (III), preferably the one or more bases, preferably the one or more solvents, and the one or more Lewis acids, the mixture subjected to reactions conditions in b) further contains an adsorbent. Preferably, one or more molecular sieves are employed as adsorbent, wherein the one or more molecular sieves preferably have a pore size of 4 Angstrom.

Surprisingly, it has been found that when the nucleophilic substitution reaction occurs in Lewis acidic conditions, the reaction is highly regio-selective for the primary OH group in 5-position 5 of the compound of formula (II), shown in the formula below:

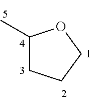

It was surprisingly found that it is not necessary is to protect any optionally present secondary OH groups in 2- or 3-position of the furanose ring when carrying out the nucleophilic substitution reaction, thereby displacing the group (Y—)ₙR₁, to obtain the compound of formula (I). Hence, with respect to the prior art synthesis disclosed in WO 2011/123668, WO 2010/135569, and WO2011/123645, the present invention provides clearly advantageous conditions which avoid the need of protecting and subsequently de-protecting secondary OH groups present on the furanose ring in the compound of formula (II).

### Lewis Acids

While there is no specific limitation with regard to their chemical nature, Lewis acids are preferred which comprise a twice positively charged ion or a three times positively charged ion. Therefore, in b), one or more Lewis acids comprising a twice positively charged ion, or one or more Lewis acids comprising a three times positively charged ion, or a combination of one or more Lewis acids comprising a twice positively charged ion and one or more Lewis acids comprising a three times positively charged ion can be employed.

Preferably, the twice positively charged metal ion and the three times positively charged metal ion is a metal ion. Therefore, it is preferred that in b), one or more Lewis acids comprising a twice positively charged metal ion, or one or more Lewis acids comprising a three times positively charged metal ion, or a combination of one or more Lewis acids comprising a twice positively charged metal ion and one or more Lewis acids comprising a three times positively charged metal ion are employed.

More preferably, the twice positively charged ion is a Zn ion, a Mg ion, a Cu ion, or an Fe ion, more preferably a Zn ion, and the three times positively charged ion is a Mn ion.

More preferably, the one or more Lewis acids is one or more of ZnBr₂, ZnCl₂, ZnI₂, MgBr₂, MgBr₂.OEt₂, CuCl₂, Cu(acetylacetonate)₂, Fe(II) fumarate, and Mn(acetylacetonate)₃.

According to the present invention, it is more preferred that the one or more Lewis acids comprises a Zn ion, wherein it is more preferred that the one or more Lewis acids is ZnBr₂.

Regarding the molar ratio of the one or more Lewis acids relative to the compound of formula (III), no specific limitation exists provided that in b), the compound of formula (I) is obtained. Preferably, in the mixture, prior to the reaction in b), the molar ratio of the one or more Lewis acids relative to the compound of formula (III) is in the range of from 0.1:1 to 5:1, more preferably in the range of from 0.2:1 to 2:1, more preferably in the range of from 0.5: 1 to 1.2:1, wherein, if more than one Lewis acid is comprised in the mixture, the molar ratio relates to the total molar amount of all Lewis acids.

Therefore, according to the present invention, it is especially preferred that according to a), a mixture is provided which comprises the compound (II), the compound (III), one or more bases and or more solvents, wherein the compound of formula (II) is the compound of formula (II-A)

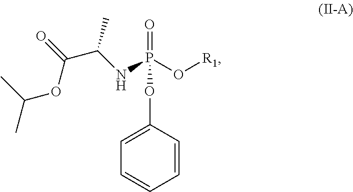

wherein R₁ is the residue of formula (IIb)

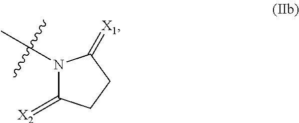

wherein both X₁ and X₂ are 0, the compound of formula (III) is the compound of formula (III-A)

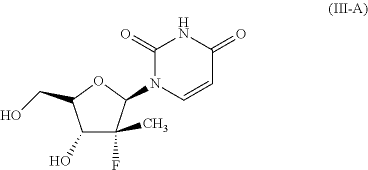

the one or more bases are selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof, the one or more solvents are selected from the group consisting of methylene chloride, methyl tert-butyl ether, tetrahydrofurane, dimethylsulphoxide, dimethylformamide, and a mixture of two or more thereof,
wherein this mixture is subjected to reaction conditions in b) in the presence of ZnBr₂ as Lewis acid, wherein prior to the reaction according to b), the molar ratio of the compound of formula (II) relative to the compound of formula (III) is in the range of from 0.9:1 to 1.2:1, the molar ratio of the Lewis acid relative to the compound of formula (III) is in the range of from 0.5:1 to 1.2:1, and the molar ratio of the one or more bases relative to the compound of formula (III) is in the range of from 0.5:1 to 1.2:1 wherein, if more than one base is comprised in the mixture provided in a), the molar ratio relates to the total molar amount of all bases.

Regarding the reaction temperature as a further reaction condition, no specific limitation exists provided that in b), the compound of formula (I) is obtained. Preferably, the reaction conditions according to b) comprise a temperature of the mixture in the range of from 0 to 80° C., more preferably in the range of from 10 to 65° C., more preferably in the range of from 20 to 50° C., such as in the range of from 20 to 30° C. or from 30 to 40° C. or from 40 to 50° C.

Regarding the pressure as a further reaction condition, no specific limitation exists provided that in b), the compound of formula (I) is obtained. Preferably, the reaction conditions according to b) comprise a pressure in the range of from 0.5 to 1.5 bar, more preferably in the range of from 0.75 to 1.25 bar, more preferably in the range of from 0.95 to 1.05 bar.

Regarding the time during which the reaction is subjected to the reaction conditions, no specific limitation exists provided that in b), the compound of formula (I) is obtained. Preferably, according to b), the mixture is subjected to the reaction conditions for a period of time in the range of from 0.5 to 48 h, more preferably in the range of from 1 to 36 h, more preferably in the range of from 2 to 24 h.

Therefore, according to the present invention, it is especially preferred that according to a), a mixture is provided which comprises the compound (II), the compound (III), one or more bases and or more solvents, wherein the compound of formula (II) is the compound of formula (II-A)

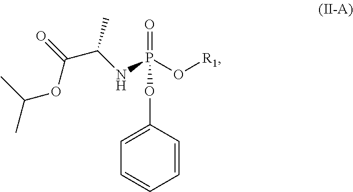

wherein R₁ is the residue of formula (IIb)

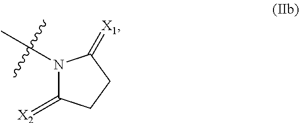

wherein both X₁ and X₂ are O, the compound of formula (III) is the compound of formula (III-A)

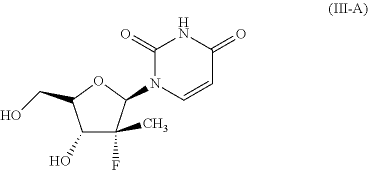

the one or more bases are selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof, the one or more solvents are selected from the group consisting of methylene chloride, methyl tert-butyl ether, tetrahydrofurane, dimethylsulphoxide, dimethylformamide, and a mixture of two or more thereof,
wherein this mixture is subjected to reaction conditions in b) in the presence of ZnBr₂ as Lewis acid,
wherein prior to the reaction according to b), the molar ratio of the compound of formula (II) relative to the compound of formula (III) is in the range of from 0.9:1 to 1.2:1, the molar ratio of the Lewis acid relative to the compound of formula (III) is in the range of from 0.5:1 to 1.2:1, and the molar ratio of the one or more bases relative to the compound of formula (III) is in the range of from 0.5:1 to 1.2:1 wherein, if more than one base is comprised in the mixture provided in a), the molar ratio relates to the total molar amount of all bases, wherein the reaction conditions according to b) comprise a temperature of the mixture in the range of from 20 to 50° C., a pressure in the range of from 0.95 to 1.05 bar, wherein the mixture is subjected to the reaction conditions for a period of time in the range of from 2 to 24 h.

Preferably, according to the present invention, in the compound of formula (II), the leaving group (Y—)ₙR₁ is either a leaving group wherein the residue R₁ is a residue of formula (A), a residue of formula (B), a residue of formula (C), a residue of formula (D) or a residue of formula (A1). Thus, it is preferred that the mixture obtained from the reaction in b) comprising the compound of formula (I) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol. It is more preferred that the mixture obtained from the reaction in b) comprising the compound of formula (I) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound substituted with one or more electron-withdrawing groups selected from the group consisting of F, Cl, Br, I, NO₂, CF₃ and a combination thereof. It is more preferred that the mixture obtained from the reaction in b) comprising the compound of formula (I) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound substituted with one or more electron-withdrawing groups.

According to the process of the present invention, a total conversion of the compound of formula (II) to the compound of formula (I) of from 40 to 100%, calculated based on the moles of the starting compound (II), was achieved. According to the process of the present invention, a total conversion of the compound of formula (II) to the compound of formula (I) of from 70 to 100%, calculated based on the moles of the starting compound (II) was achieved when the reaction of the invention was performed using ZnBr₂ as Lewis acid in Et₃N. According to the process of the present invention, a total conversion of the compound of formula (II) to the compound of formula (I) of from 90 to 100%, calculated based on the moles of the starting compound (II) was achieved when the reaction of the invention was performed using ZnBr₂ as Lewis acid in DBU. According to the process of the present invention, a total conversion of the compound of formula (II) to the compound of formula (I) of from 90 to 100%, calculated based on the moles of the starting compound (II) was achieved when the reaction of the invention was performed using ZnCl₂ as Lewis acid in Et₃N.

The correct regio-isomer on the primary OH group of furanose ring is formed with a regiose-lectivity of from 80 to 100%, calculated with respect to the total conversion of the starting compound of formula (II).

### Compound of Formula (I)

According to the present invention, the mixture obtained in (i) comprises the compound of formula (I). The compound of formula (I) comprises several stereogenic atoms, including the phosphorous atom. According to the present invention a compound of formula (I) generally includes all isomers, stereoisomers, enantiomers and diastereomers thereof. The preferred compound of formula (I) is an isomer wherein the stereogenic centers of the compound of formula (I) are as disclosed in formula (I′)

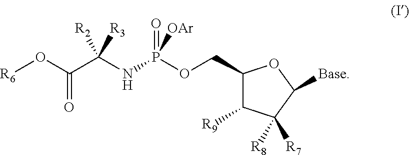

According to the present invention, the chirality of each stereogenic center can be assigned according to the Cahn-Ingold-Prelog system (CIP).

In accordance with the preferred compounds of formulas (II) and (III) described above, the compound of formula (I) is preferably the compound of formula (I′)

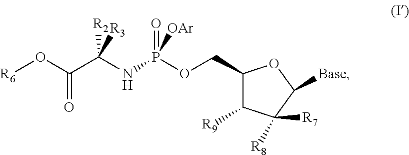

wherein Base a residue of formula (B1)

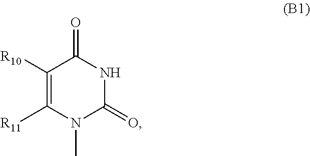

R₁₀ and R₁₁ are independently H or optionally substituted C₁-C₆ alkyl, wherein more preferably, R₁₀ and R₁₁ are H,
one of the two residues R₇ and R₈ is F, the other residue of R₇ and R₈ is methyl, R₉ is OH, R₆ is isopropyl, one of the residues R₂ and R₃ is H and the other is methyl such that the chirality according to the Cahn-Ingold-Prelog (CIP) system of the carbon atom bearing R₂ and R₃ is S, and Ar is phenyl.

More preferably, the compound of formula (I) is the compound of formula (I-A)

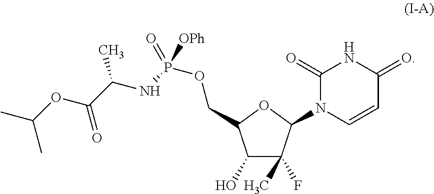

### Step c)

Preferably, the compound of formula (I) is separated from the mixture obtained in b), and the process of the present invention further comprises

c) separating the compound of formula (I) from the mixture obtained in step b).

More preferably, the separating according to c) comprises crystallizing the compound of formula (I) which is preferably obtained as amorphous compound from the reaction in b). It is preferred that after crystallization, the crystallized compound of formula (I) is separated from its mother liquor. Therefore, the present invention also relates to the above-defined process, comprising
c1) crystallizing the compound of formula (I) in the mixture obtained in b), obtaining the crystallized compound of formula (I) in its mother liquor;
c2) separating the compound of formula (I) from its mother liquor.

Prior to crystallizing according to c1), it is preferred that the compound of formula (I) is separated from the liquid phase of the mixture obtained in b), preferably including filtration or centrifugation, more preferably filtration. Further, prior to c1), it is preferred that the solid compound of formula (I) obtained from filtration or centrifugation, preferably filtration, is washed and/or dried, preferably washed and dried. No specific limitation exists for the washing agent. Preferred washing agents include isopropyl acetate. No specific limitation exist for the drying conditions. Preferred drying conditions include a pressure below 1 bar, preferably drying in vacuo. Further prior to (c1), the thus preferably obtained solid compound of formula (I) is dissolved in one or more solvents, including, for example, toluene and isopropyl acetate.

Further prior to crystallization according to c1), the thus dissolved compound of formula can be subjected to extraction, including, for example, extraction with aqueous sodium chloride, obtaining an organic phase from which the solvent is preferably removed whereafter the solid compound of formula (I) is dissolved in one or more further solvents. If extraction is carried, it is, for example, preferred to dissolve the compound of formula (I), after separation from the liquid phase of the mixture obtained in b) and drying, in a first organic solvent, for example, isopropyl acetate, subject the thus obtained solution to extraction, for example with aqueous sodium chloride, obtaining an organic phase from which the solvent is suitably removed, and dissolve the thus obtained solid compound of formula (I) in a second organic solvent, for example toluene.

During crystallization in c1), it can be preferred that suitable seed crystals are added, preferably seed crystals of the compound of formula (I).

After the crystallization in c1) from which the crystallized compound of formula (I) is obtained in its mother liquor, the crystallized compound of formula (I) is preferably suitably separated from its mother liquor, for example by filtration or centrifugation. The thus separated crystallized compound of formula (I) can be subjected to washing, wherein preferred washing agents include methyl tert-butyl ether, dichloro methane and mixtures thereof, and subject the optionally washed crystallized compound of formula (I) to drying. Preferred drying conditions include temperatures in the range of from 10 to 60° C., preferably in the range of from 30 to 50° C., and a pressure below ambient pressure.

Therefore, the separating in c) preferably comprises
c1) crystallizing the compound of formula (I) in the mixture obtained in b), obtaining the crystallized compound of formula (I) in its mother liquor, the crystallizing optionally comprising seeding;
c2) separating the compound of formula (I) from its mother liquor, comprising
   c21) subjecting the mother liquor comprising the crystallized compound of formula (I) to filtration;
   c22) optionally washing the filter cake comprising the compound of formula (I);
   c23) drying the optionally washed filter cake comprising the compound of formula (I).

Further, as discussed above, the separating in c) may comprise
c01) separating the compound of formula (I) from the liquid phase of the mixture obtained in b), preferably comprising
   c011) subjecting the mixture obtained in b) comprising the compound of formula (I) to filtration;
   c012) optionally washing the filter cake comprising the compound of formula (I);
   c013) drying the optionally washed filter cake comprising the compound of formula (I);
c02) dissolving the dried filter cake in one or more organic solvents;
c1) crystallizing the compound of formula (I) in the solution obtained in c02), obtaining the crystallized compound of formula (I) in its mother liquor, the crystallizing optionally comprising seeding;
c2) separating the compound of formula (I) from its mother liquor, comprising
   c21) subjecting the mother liquor comprising the crystallized compound of formula (I) to filtration;
   c22) optionally washing the filter cake;
   c23) drying the optionally washed filter cake.

Yet further, as discussed above, the separating in c) may comprise
c01) separating the compound of formula (I) from the liquid phase of the mixture obtained in b), preferably comprising
   c011) subjecting the mixture obtained in b) comprising the compound of formula (I) to filtration;
   c012) optionally washing the filter cake comprising the compound of formula (I);
   c013) drying the optionally washed filter cake comprising the compound of formula (I);
c02) dissolving the dried filter cake in one or more organic solvents;
c03) subjecting the solution obtained in c02) to extraction, preferably comprising
   c031) extraction, preferably with aqueous sodium chloride, obtaining an organic phase comprising the compound of formula (I);
   c032) removing the solvent from the organic phase obtained in c031);
c04) dissolving the compound of formula (I) obtained in c032) in one or more solvents;
c1) crystallizing the compound of formula (I) in the solution obtained in c04), obtaining the crystallized compound of formula (I) in its mother liquor, the crystallizing optionally comprising seeding;
c2) separating the compound of formula (I) from its mother liquor, comprising
   c21) subjecting the mother liquor comprising the crystallized compound of formula (I) to filtration;
   c22) optionally washing the filter cake;
c23) drying the optionally washed filter cake.

According to the present invention, it is preferred that the composition obtained from c) or c2) has a content of the one or more Lewis acids comprising a twice positively charged ion or three times positively charged ion, preferably a twice positively charged ion, more preferably the Zn ion, of at most, preferably less than 1350 weight-ppm, based on the total weight of the composition and calculated based on the weight of the twice positively charged ion or three times positively charged ion, preferably the twice positively charged ion, more preferably the Zn ion, comprised in the one or more Lewis acids, wherein, in case the composition comprises more than one Lewis acid, said weight-ppm values relate to each individual Lewis acid, wherein said content is more preferably at most, preferably less than, 600 weight-ppm, more preferably at most, preferably less than, 100 weight-ppm. According to the present invention, the M²⁺ content, preferably the Zn²⁺ content is measured with a ICP-MS (Inductively coupled plasma mass spectrometry) instrument, preferably the M²⁺ content, preferably the Zn²⁺ content is measured after crystallization.

### Process for Preparing the Compound of Formula (II)

According to the present invention, it is preferred that the compound of formula (II) is prepared from a compound of formula (IV)

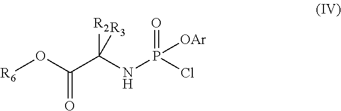

and a compound R₁(—Y)ₙH. Therefore, the present invention also relates to the process as defined above, further comprising providing the mixture according to a) by a process comprising
(i) providing a mixture comprising a compound of formula (IV)

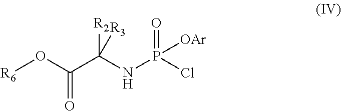

and a compound R₁(—Y)ₙH;
(ii) subjecting the mixture provided in (i) to reaction conditions, obtaining a mixture comprising the compound of formula (II).

Further, the present invention also relates to a process for the preparation of a compound of formula (II)

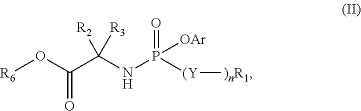

the process comprising
(i) providing a mixture comprising a compound of formula (IV)

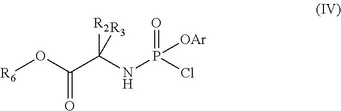

and a compound R₁(—Y)ₙH;
(ii) subjecting the mixture provided in (i) to reaction conditions, obtaining a mixture comprising the compound of formula (II).

Regarding the definitions and preferred definitions of the R₂, R₃, R₆, Ar, n and Y, reference is made to the discussions above and the embodiment section hereinunder.

### Step i)

Generally, the compound of formula (IV) can be prepared by all conceivable and suitable processes. For example, with regard to a preferred compound of formula (IV), wherein R₆ is isopropyl, one of the residues R₂ and R₃ is H and the other is methyl, and Ar is Ar is phenyl, reference can be made to J. Org. Chem. 2011, 76, 8311.

In compound R₁(—Y)ₙH, R¹, Y and n are as defined above, in any embodiments and preferred embodiments. Generally, the compound of formula R₁(—Y)ₙH can be prepared by all conceivable and suitable processes. For example, with regard to a preferred compound of R₁(—Y)ₙH, wherein n is 1, Y is O, and R₁ is the residue of formula (IIb)

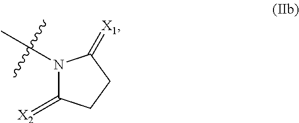

wherein both X₁ and X₂ are O, reference can be made to Tetrahedron Letters 1987, 28, 2375.

Regarding the molar ratio of the compound of formula (IV) relative to the compound R₁(—Y)ₙH, no specific limitation exists provided that in ii), the compound of formula (II) is obtained. Preferably, in the mixture provided in i), prior to the reaction in ii), the molar ratio of the compound of formula (IV) relative to the compound R₁(—Y)ₙH is in the range of from 0.5:1 to 2:1, more preferably in the range of from 0.7:1 to 1.3:1, more preferably in the range of from 0.9: to 1.1:1.

According to the present invention, it is preferred that the mixture provided in i) comprises, in addition to the compound of formula (IV) and the compound R₁(—Y)ₙH, one or more solvents. Preferably, the one or more solvents are organic solvents. More preferably, the one or more organic solvents are aprotic organic solvents. More preferably, the one or more solvents are selected from the group consisting of methylene chloride, methyl tert-butyl ether, tetrahydrofurane, dimethylsulphoxide, dimethylformamide, and a mixture of two or more thereof.

According to the present invention, it is preferred that the mixture provided in i) comprises, in addition the compound of formula (IV) and the compound R₁(—Y)ₙH, and preferably in addition to the one or more solvents, one or more bases. No specific limitation exists with regard to the chemical nature of the one or more bases provided that the reaction according to ii) can be carried out, preferably in the one or more solvents mentioned above. Preferably, the one or more bases are organic bases. More preferably, the one or more bases are selected from the group consisting of an amine, an amidine, a heteroaromatic compound comprising a basic ring-nitrogen atom, and a mixture of two or more thereof. More preferably, the one or more bases are selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof.

Therefore, according to the present invention, it is preferred that the mixture provided in i) comprises in addition to the compound of formula (IV) and the compound R₁(—Y)ₙH, one or more solvents which are preferably selected from the group consisting of methylene chloride, methyl tert-butyl ether, tetrahydrofurane, dimethylsulphoxide, dimethylformamide, and a mixture of two or more thereof, and one or more bases which are preferably selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof.

Regarding the molar ratio of the one or more bases relative to the compound R₁(—Y)ₙH, no specific limitation exists provided that in ii), the compound of formula (II) is obtained. Preferably, in the mixture provided in i), prior to the reaction in ii), the molar ratio of the one or more bases relative to the compound R₁(—Y)ₙH is in the range of from 0.05:1 to 5:1, more preferably in the range of from 0.1:1 to 2:1, more preferably in the range of from 0.5:1 to 1.2:1. If more than one base is comprised in the mixture provided in i), the molar ratio relates to the total molar amount of all bases.

### Step ii)

Regarding the reaction temperature as a further reaction condition, no specific limitation exists provided that in ii), the compound of formula (II) is obtained. Preferably, the reaction conditions according to ii) comprise a temperature of the mixture in the range of from 0 to 30° C., more preferably in the range of from 0 to 20° C., more preferably in the range of from 0 to 10° C.

Regarding the pressure as a further reaction condition, no specific limitation exists provided that in b), the compound of formula (I) is obtained. Preferably, the reaction conditions according to b) comprise a pressure in the range of from 0.5 to 1.5 bar, more preferably in the range of from 0.75 to 1.25 bar, more preferably in the range of from 0.95 to 1.05 bar.

Regarding the time during which the reaction is subjected to the reaction conditions, no specific limitation exists provided that in b), the compound of formula (I) is obtained. Preferably, according to b), the mixture is subjected to the reaction conditions for a period of time in the range of from 0.5 to 48 h, more preferably in the range of from 1 to 36 h, more preferably in the range of from 2 to 24 h.

Step iii)

Preferably, according to the present invention, the compound of formula (II) comprised in the mixture obtained in ii) is separated from the mixture. Therefore, the process of the present invention preferably further comprises

iii) separating the compound of formula (II) from the mixture obtained from ii).

During the separating according to iii), the compound of formula (II) is preferably crystallized. More preferably, the thus crystallized compound is suitably separated from its mother liquor. Therefore, the separating of the process of the present invention preferably further comprises
iii1) crystallizing the compound of formula (II) in the mixture obtained in (ii), obtaining the crystallized compound of formula (II) in its mother liquor; and
iii2) separating the compound of formula (II) from its mother liquor, preferably by filtration.

Preferably, the separating in iii) comprises
iii1) crystallizing the compound of formula (II) in the mixture obtained in ii), obtaining the crystallized compound of formula (II) in its mother liquor, the crystallizing optionally comprising seeding;
iii2) separating the compound of formula (II) from its mother liquor, comprising
   iii21) subjecting the mother liquor comprising the crystallized compound of formula (II) to filtration;
   iii22) optionally washing the filter cake;
   iii23) drying the optionally washed filter cake.

Preferably, prior to crystallization according to iii1), the mixture obtained in ii) comprising the compound of formula (II) is subjected to extraction including, for example, extraction with water, obtaining an organic phase from which the solvent is preferably removed whereafter the solid compound of formula (II) is dissolved in one or more further solvents, preferably comprising methyl tert-butyl ether.

During crystallization in iii1), it can be preferred that suitable seed crystals are added, preferably seed crystals of the compound of formula (II). Further, during crystallization, at least two sequences of dissolving with subsequent crystallizing can be carried out.

After the crystallization in iii1) from which the crystallized compound of formula (II) is obtained in its mother liquor, the crystallized compound of formula (II) is preferably suitably separated from its mother liquor, for example by filtration or centrifugation. The thus separated crystallized compound of formula (II) can be subjected to washing, wherein preferred washing agents include methyl tert-butyl ether, dichloro methane and mixtures thereof, and subject the optionally washed crystallized compound of formula (II) to drying. Preferred drying conditions include temperatures in the range of from 10 to 60° C., preferably in the range of from 30 to 50° C., and a pressure below ambient pressure.

Therefore, the present invention also relates to above-defined process, wherein the separating in iii) comprises
iii0) subjecting the mixture obtained in ii) to extraction, preferably comprising
   iii01) extraction, preferably with water, obtaining an organic phase comprising the compound of formula (II);
   iii02) removing the solvent from the organic phase obtained in iii01);
   iii03) dissolving the solid obtained in iii02) in one or more solvents;
iii1) crystallizing the compound of formula (II) in the mixture obtained in iii03), obtaining the crystallized compound of formula (II) in its mother liquor, the crystallizing optionally comprising seeding;
iii2) separating the compound of formula (II) from its mother liquor, comprising
   iii21) subjecting the mother liquor comprising the crystallized compound of formula (II) to filtration;
   iii22) optionally washing the filter cake;
   iii23) drying the optionally washed filter cake.

### Compounds, Compositions, and Mixtures

### Compound of Formula (II)

Yet further, the present invention provides a compound of formula (II)

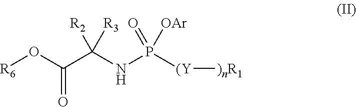

wherein
Ar is phenyl, naphtyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl) and CN; R₆ is C₁-C₆ alkyl or C₃-C₁₀ cycloalkyl optionally substituted with at least one of C₁-C₆ alkyl and aryl;
(Y—)ₙR₁ is a leaving group for nucleophilic substitution reaction, wherein n is 0 or 1 and wherein Y is O, N or S;
wherein, when n is 1,
R₁ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups, more preferably phenyl substituted with one or more electron-withdrawing groups, wherein the one or more electron-withdrawing groups are preferably F, Cl, Br, I, or NO₂; or
R₁ is a residue of formula (A)

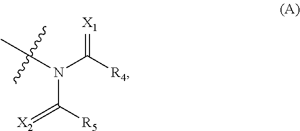

a residue of formula (B)

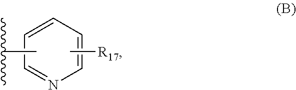

a residue of formula (C)

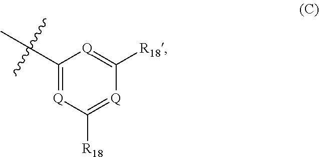

or a residue of formula (D)

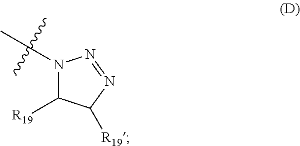

or wherein, when n is 0,
R₁ is a residue of formula (A1)

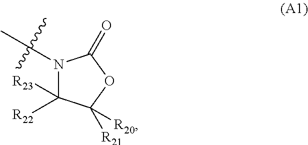

wherein at each occurrence
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.

Preferred compounds of formula (II) according to the present invention are compounds wherein n is 1 and the leaving group is Y—R₁ wherein R₁ is a residue selected from the group consisting of a residue of formula (A), a residue of formula ((B), a residue of formula (C) and a residue of formula (D) as defined above. Preferred compounds of formula (II) according to the present invention are also compounds wherein n is 0 and the leaving group is Y—R₁ wherein R₁ is the residue of formula (A1) as defined above.

Preferred compounds of formula (II) according to the present invention are compounds wherein n is 1 and the leaving group is Y—R₁ wherein R₁ is a residue formula (A) as defined above.

Preferred compounds of formula (II) according to the present invention are compounds wherein n is 1 and the leaving group is Y—R₁ wherein R₁ is a residue formula (B) as defined above.

Preferred compounds of formula (II) according to the present invention are compounds wherein n is 1 and the leaving group is Y—R₁ wherein R₁ is a residue formula (C) as defined above.

Preferred compounds of formula (II) according to the present invention are compounds wherein n is 1 and the leaving group is Y—R₁ wherein R₁ is a residue formula (D) as defined above.

Preferred compounds of formula (II) according to the present invention are compounds wherein n is 0 and the leaving group is Y—R₁ wherein R₁ is a residue formula (A1) as defined above.

Preferred compounds of formula (II) according to the present invention are compounds wherein n is 1 and the leaving group is Y—R₁ wherein R₁ is the residue of formula (IIc). It is more preferred a compound of formula (II) wherein n is 1 and the leaving group is O—R₁ wherein R₁ is the residue of formula (IIc).

Preferred compounds of formula (II) according to the present invention are compounds wherein n is 1 and the leaving group is Y—R₁ wherein R₁ is the residue of formula (IIb). It is more preferred a compound of formula (II) wherein n is 1 and the leaving group is O—R₁ wherein R₁ is the residue of formula (IIb).

Preferred compound of formula (II-I) according to the present invention are compounds having the stereochemistry as specified of formula (II′-I)

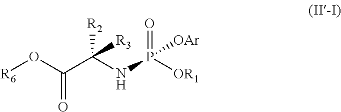

wherein R₁, R₂, R₃, R₆ and Ar are as defined for formula (II-I).

Preferred compounds of formula (II) or (II′-I) according to the present invention are compounds wherein R₁ is a residue of formula (A)

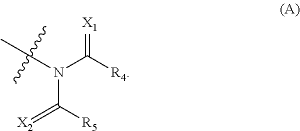

Regarding the chemical nature of X₁, X₂, R₄ and R₅, no specific limitation exists, provided that the leaving group is suitable for the above-described reaction.

Preferably, X₁ and X₂ are independently O or S. More preferably, both X₁ and X₂ are O.

The residues R₄ and R₅ can be either individual residues or can be connected to form a ring structure, preferably to a 5-, 6-, or 7-membered ring structure. This ring structure can be fused, in turn, to at least one further ring, preferably one further ring, preferably a 5- or 6-membered. If R₄ and R₅ are individual residues, R₄ and R₅ are preferably independently H, OH, NH₂, C₁-C₆ alkyl or C₁-C₆ alkoxy. If R₄ and R₅ are connected to form a ring structure, it is preferred that R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S. If R₄ and R₅ are connected to form a ring structure, it is more preferred that R₄ and R₅, together with the structure —C—N—C— according to formula (A), form an optionally substituted, 5-membered saturated ring, wherein said ring is optionally fused to a 5- or 6-membered, optionally substituted ring which is a C₅-C₆ cycloalkyl, an aryl or a heterocycle comprising one or more heteroatoms independently being N, O or S. If R₄ and R₅ are connected to form a ring structure, it is more preferred that R₄ and R₅, together with the structure —C—N—C— according to formula (A), form a 5-membered saturated ring, wherein said ring is optionally fused to a 6-membered, optionally substituted ring which is an aromatic ring, wherein the aromatic ring is preferably a benzo structure.

Preferred compounds of formula (II) or (II′-I) according to the present invention are compounds wherein R₁ is a residue of formula (IIc)

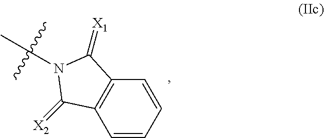

wherein both X₁ and X₂ are preferably O. More preferably, the leaving group is O—R₁ wherein R₁ is the residue of formula (IIc).

Preferred compounds of formula (II) or (II′-I) according to the present invention are compounds wherein R₁ is a residue of formula (IIb)

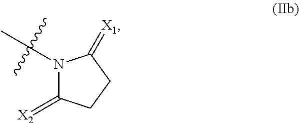

wherein both X₁ and X₂ are preferably O. More preferably, the leaving group is O—R₁ wherein R₁ is the residue of formula (IIb).

Preferred compounds of formula (II) or (II′-I) according to the present invention are compounds wherein R₁ is a residue of formula in case n is 1, R₁ is a residue of formula (B)

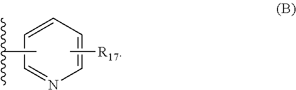

Regarding the chemical nature of R₁₇, no specific limitation exists, provided that the leaving group is suitable for the above-described reaction. Preferably, R₁₇ is an electron-withdrawing group, more preferably R₁₇ is selected from the group consisting of F, Cl, Br, I, NO₂, CHO, COOH, COO—(C₁-C₆)alkyl, CN, and COCl, more preferably F, Cl, NO₂, more preferably NO₂. It is also possible that more than one electron-withdrawing groups R₁₇ can be present in the residue of formula (B), for example 2 or 3 electron-withdrawing groups R₁₇. If more than one electron-withdrawing group R₁₇ are present in the residue of formula (B), they can be of iden-tical or different chemical nature and are preferably from the group described above. If one electron-withdrawing groups R₁₇ are present, which is preferred, R₁₇ is preferably in position meta with respect to the nitrogen atom of the pyridine ring.

Generally, the pyridine residue (B) can be attached to Y in 2-, 3-, or 4-position of the pyridine ring, preferably in 2-position.

More preferably, the leaving group is O—R₁ wherein R₁ is the residue of formula (B).

Preferred compounds of formula (II) or (II′-I) according to the present invention are compounds wherein R₁ is a residue of formula (C)

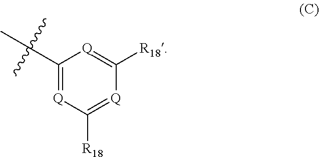

Regarding the chemical nature of Q, R₁₈ and R_{18′}, no specific limitation exists, provided that the leaving group is suitable for the above-described reaction. Preferably, R₁₈ and R_{18′} are independently selected from the group consisting of F, Cl, Br, I, or C₁-C₆ alkoxy, preferably methoxy. Preferably, each Q is independently C or N, wherein at least one Q, such as one Q, two Qs, or three Qs, is N. More preferably, the leaving group is O—R₁ wherein R₁ is the residue of formula (C).

Preferred compounds of formula (II) or (II′-I) according to the present invention are compounds wherein R₁ is a residue of formula (D)

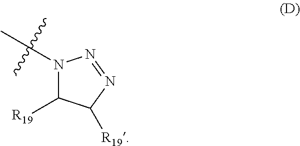

Regarding the chemical nature of Q, R₁₉ and R_{19′}, no specific limitation exists, provided that the leaving group is suitable for the above-described reaction. The residues R₁₉ and R_{19′} can be either individual residues or can be connected to form a ring structure, preferably to a 5-, 6-, or 7-membered ring structure. This ring structure can be fused, in turn, to at least one further ring, preferably one further ring, preferably a 5- or 6-membered.

If R₁₉ and R_{19′} are individual residues, R₁₉ and R_{19′} are preferably independently H, OH, NH₂, C₁-C₆ alkyl optionally substituted with at least one of OH and NH₂, or C₁-C₆ alkoxy optionally substituted with at least one of OH and NH₂.

If R₁₉ and R_{19′} are connected to form a ring structure, it is preferred that R₁₉ and R_{19′}, together with the structure —C—C— according to formula (D), form an optionally substituted, 5-, 6-, or 7-membered saturated or partially unsaturated ring or aromatic ring, wherein said ring is optionally fused to a 5- or 6-membered, optionally substituted ring which is a C₅-C₆ cycloalkyl, an aryl, preferably benzo, or a heterocycle comprising one or more heteroatoms independently being N, O or S. If R₁₉ and R_{19′} are connected to form a ring structure, it is more preferred that R₁₉ and R_{19′}, together with the structure —C—C— according to formula (D), form an optionally substituted, 5-, 6-, or 7-membered saturated or partially unsaturated ring or an aromatic ring, wherein the aromatic ring is preferably benzo, wherein said ring is optionally fused to a 5- or 6-membered, optionally substituted ring which is a heterocycle comprising one or more heteroatoms independently being N, O or S.

The substituents of the optionally substituted, 5-, 6-, or 7-membered saturated or partially unsaturated ring or aromatic ring are selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, wherein aryl is preferably phenyl, heteroaryl. The substituent when present is at least one substituent, preferably one substituent.

If R₁₉ and R_{19′} are connected to form a benzo structure, it is preferred that the benzo is substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, wherein aryl is preferably phenyl, heteroaryl.

More preferably, the leaving group is O—R₁ wherein R₁ is the residue selected from the group consisting of a residue of formula (IIb), of formula (IIc) or of formula (D).

Preferred compounds of formula (II) or (II′-I) according to the present invention are compounds wherein for n=0, R₁ is a residue of formula (A1)

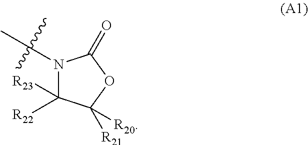

Regarding the chemical nature of R₂₀, R₂₁, R₂₂ and R₂₃, no specific limitation exists, provided that the leaving group is suitable for the above-described reaction. The residues R₂₀, R₂₁, R₂₂ and R₂₃ can be individual residues. Alternatively, two of these residues are individual residues, and two of these residues are connected to form a ring structure.

If these residues are individual residues, R₂₀, R₂₁, R₂₂ and R₂₃ are preferably each independently H, aryl, or C₁-C₆ alkyl optionally substituted with at least one of C₁-C₆ alkoxy optionally substituted with at least one of OH and NH₂, more preferably aryl or alkyl. Preferably, if R₂₂ and R₂₃ are individual residues are each independently H, aryl, or C₁-C₂ or C₄-C₆ alkyl optionally substituted with at least one of C₁-C₆ alkoxy optionally substituted with at least one of OH and NH₂. More preferably, if R₂₂ and R₂₃ are individual residues are each independently H, aryl, or C₁-C₆ alkyl substituted with at least one of C₁-C₆ alkoxy optionally substituted with at least one of OH and NH₂.

If two of these residues are individual residues, and two of these residues are connected to form a ring structure, it is preferred that R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl, and the other two residues, R₂₁ and R₂₃, or R₂₁ and R₂₂, or R₂₀ and R₂₃, or R₂₀ and R₂₂, which do not form the ring structure, are preferably each independently H, aryl, or C₁-C₆ alkyl optionally substituted with at least one of C₁-C₆ alkoxy optionally substituted with at least one of OH and NH₂.

The substituents of the optionally substituted 5-, 6-, or 7-membered saturated or partially unsaturated or aromatic ring which is an aryl, preferably benzo, or a heterocycle comprising one or more heteroatoms independently being N, O or S are selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, wherein aryl is preferably phenyl, heteroaryl. The substituent when present is at least one substituent, preferably one substituent.

If R₂₀ and R₂₂, or R₂₀ and R₂₃, or R₂₁ and R₂₂, or R₂₁ and R₂₃ are connected to form a benzo structure, it is preferred that the benzo is substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, wherein aryl is preferably phenyl, heteroaryl.

Most preferred compounds of formula (II) or (II′-I) according to the present invention are compounds wherein R₁ is the residue of formula (IIc). It is preferred that in the residue of formula (IIc) X₁ is O and X₂ is O.

Most preferred compounds of formula (II) or (II′-I) according to the present invention are compounds wherein R₁ is the residue of formula (IIb). It is preferred that in the residue of formula (IIb) X₁ is O and X₂ is O.

More preferred compounds of formula (II) according to the present invention are compounds of formula (II-A)

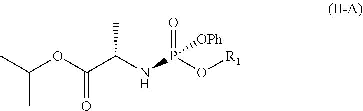

or of formula (II-B)

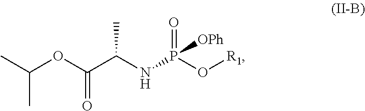

wherein R₁ is as defined above, wherein preferably, R₁ is a residue selected from the group consisting of a residue of formula (A), wherein, more preferably, R₁ is a residue of formula (IIb)

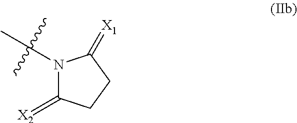

or of formula (IIc)
or

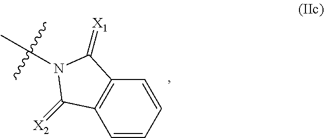

wherein X₁ is O and X₂ is O.

It is further preferred that in formula (II-A) or (II-B), R₁ is the residue of formula (B) as defined above in connection with compounds of formula (II′-1).

It is further preferred that in formula (II-A) or (II-B), R₁ is a residue of formula (C) as defined above in connection with compounds of formula (II′-1).

It is further preferred that in formula (II-A) or (II-B), R₁ is a residue of formula (D) as defined above in connection with compounds of formula (II′-1).

It is most preferred that in formula (II-A) or (II-B) R₁ is a residue of formula (IIc).

### Mixture Comprising a Compound of Formula (I)

Further, the present invention provides a mixture comprising a compound of formula (I) including all isomers, stereoisomers, enantiomers and diastereomers thereof

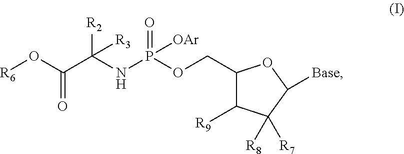

and salts thereof, wherein R₆, R₂, R₃, R₇, R₈, R₉, Ar and Base are as defined above, preferably comprising a compound of formula (I′)

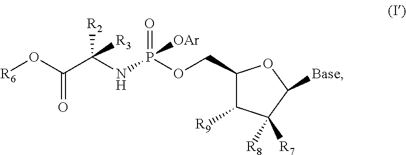

and salts thereof, more preferably comprising a compound of formula (I-A)

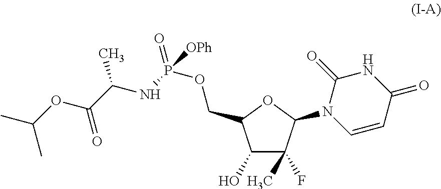

and salts thereof, which mixture is obtainable or obtained by the process of the present invention, preferably by the process of the present comprising the separating according to c). Preferably, the mixture is obtainable or obtained according to the process of the invention wherein compound (II) is reacted with compound (III) in the presence of one or more Lewis acids according to steps a) and b) as defined above.

Preferably, the mixture is obtainable or obtained according to the process of the invention wherein in compound (II) n is 1 and the leaving group is Y—R₁ wherein R₁ is a residue selected from the group consisting of a residue of formula (A) as defined above in connection with compound (II′-1).

Preferably, the mixture is obtainable or obtained according to the process of the invention wherein in compound (II) n is 1 and the leaving group is Y—R₁ wherein R₁ is a residue selected from the group consisting of a residue of formula (B) as defined above in connection with compound (II′-1).

Preferably, the mixture is obtainable or obtained according to the process of the invention wherein in compound (II) n is 1 and the leaving group is Y—R₁ wherein R₁ is a residue selected from the group consisting of a residue of formula (C) as defined above in connection with compound (II′-1).

Preferably, the mixture is obtainable or obtained according to the process of the invention wherein in compound (II) n is 1 and the leaving group is Y—R₁ wherein R₁ is a residue selected from the group consisting of a residue of formula (D) as defined above in connection with compound (II′-1).

Preferably, the mixture is obtainable or obtained according to the process of the invention wherein in compound (II) n is 0 and the leaving group is Y—R₁ wherein R₁ is a residue selected from the group consisting of a residue of formula (A1) as defined above in connection with compound (II′-1).

It is also preferred that in compound (II) n is 1 and the leaving group is Y—R₁ wherein R₁ is the residue of formula (IIc). It is more preferred that in the process in compound (II) n is 1 and the leaving group is O—R₁ wherein R₁ is the residue of formula (IIc). It is further more preferred that n is 1 and that the leaving group is Y—R₁ wherein R₁ is the residue of formula (IIb). It is more preferred that n is 1 and that the leaving group is O—R₁ wherein R₁ is the residue of formula (IIb).

According to the present invention, the mixture comprising a compound of formula (I) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol. It is more preferred that the mixture obtained from the reaction in b) comprising the compound of formula (I) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound substituted with one or more electron-withdrawing groups selected from the group consisting of F, Cl, Br, I, NO₂, CF₃ and a combination thereof. It is more preferred that the mixture obtained from the reaction in b) comprising the compound of formula (I) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound substituted with one or more electron-withdrawing groups.

According to the present invention, the mixture comprising a compound of formula (I) has a content, based on the total weight of the mixture of less than 100 weight-ppm, preferably less than 50 weight-ppm, more preferably less than 10 weight-ppm, even more preferably less than 5 weight-ppm of an aryl-OH that is 4-nitrophenol.

According to the present invention, the mixture comprising a compound of formula (I) has a content, based on the total weight of the mixture of less than 100 weight-ppm, preferably less than 50 weight-ppm, more preferably less than 10 weight-ppm, even more preferably less than 5 weight-ppm of an aryl-OH that is penta-fluorophenol.

The present invention is advantageous with respect to the prior art processes for example disclosed in international patent applications WO2011/123672 and WO 2014/047117 where for preparing the compound of formula (I) and in particular sofosbuvir of formula (I-A), aryloxide leaving groups are used substituted with one or more electron withdrawing groups which are considered toxic by the Food and Drug administration if present above a certain threshold in a product. The present invention provides a simple and effective synthesis which does not necessarily entail the use of aryloxide leaving groups substituted one or more elec-tron withdrawing groups. In fact, the present invention preferably provides non-toxic leaving groups (Y—)ₙR₁ wherein R₁ is selected from the groups consisting of a residue of formulas (A) to (D) and (A1) as defined above.

### Mixture Comprising a Compound of Formula (II)

Further, the present invention provides a mixture comprising a compound of formula (II)

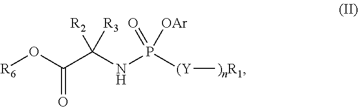

preferably comprising a compound of formula (II′)

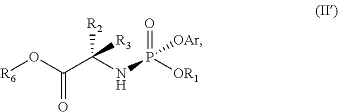

more preferably comprising a compound of formula (II-A)

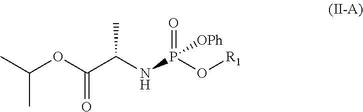

or a mixture comprising a compound of formula (II-B)

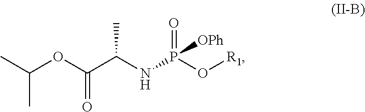

wherein R₁, R₂, R₃, R₆ and Ar are as defined above, and wherein R₁ is preferably a residue selected from the group of consisting of a residue of formula (A), a residue of formula (B), a residue of formula (C) and residue of formula (D) as defined above, more preferably is a residue of formula (IIb)

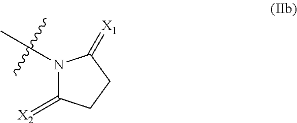

or of formula (IIc)
or

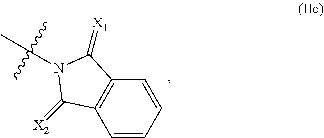

wherein X₁ is O and X₂ is O;
which mixture is obtainable or obtained by the process of the present invention, preferably by the process of the present invention comprising the a) and b).

It is further preferred that in the mixture in the compound of formula (II), (II′), (II-A) or (II-B), R₁ is the residue of formula (A) as defined above in connection with compounds of formula (II′-1).

It is further preferred that in the mixture in the compound of formula (II), (II′), (II-A) or (II-B), R₁ is the residue of formula (B) as defined above in connection with compounds of formula (II′-1).

It is further preferred that in the mixture in the compound of formula (II), (II′), (II-A) or (II-B), R₁ is a residue of formula (C) as defined above in connection with compounds of formula (II′-1).

It is further preferred that in the mixture in the compound of formula (II), (II′), (II-A) or (II-B), R₁ is a residue of formula (D) as defined above in connection with compounds of formula (II′-1).

It is further preferred that in the mixture in the compound of formula (II), R₁ is a residue of formula (A1) as defined above in connection with compounds of formula (II′-1).

It is most preferred that in the mixture in the compound of formula (II), (II′), (II-A) or (II-B), is a residue of formula (IIc).

According to the present invention, the mixture comprising a compound of formula (II) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol. It is more preferred that the mixture obtained from the reaction in a) comprising the compound of formula (II) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound substituted with one or more electron-withdrawing groups selected from the group consisting of F, Cl, Br, I, NO₂, CF₃ and a combination thereof. It is more preferred that the mixture obtained from the reaction in b) comprising the compound of formula (II) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound substituted with one or more electron-withdrawing groups.

According to the present invention, the mixture comprising a compound of formula (II) has a content, based on the total weight of the mixture of less than 100 weight-ppm, preferably less than 50 weight-ppm, more preferably less than 10 weight-ppm, even more preferably less than 5 weight-ppm of an aryl-OH that is 4-nitrophenol.

According to the present invention, the mixture comprising a compound of formula (II) has a content, based on the total weight of the mixture of less than 100 weight-ppm, preferably less than 50 weight-ppm, more preferably less than 10 weight-ppm, even more preferably less than 5 weight-ppm of an aryl-OH that is penta-fluorophenol.

### Composition Comprising a Compound of Formula (I)

Further, the present invention provides a composition of which at least 99.90 weight-%, preferably at least 99.92 weight-% based on the weight of the composition, consist of the compound of formula (I) including isomers, stereoisomers, enantiomers, diastereomers thereof

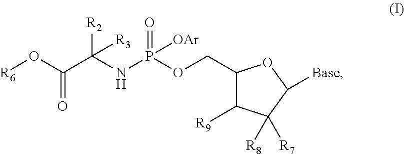

and salts thereof, wherein R₆, R₂, R₃, R₇, R₈, R₉, Ar and Base are as defined above, preferably consists of the compound of formula (I′) including isomers, stereoisomers, enantiomers, diastereomers thereof

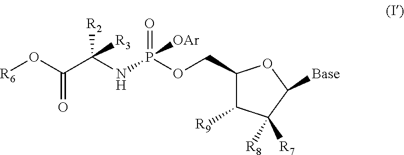

and salts thereof, more preferably consist of the compound of formula (I-A)

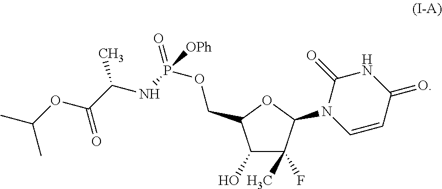

According to the present invention, the composition comprising a compound of formula (I) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol. It is more preferred that the mixture obtained from the reaction in a) comprising the compound of formula (II) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound substituted with one or more electron-withdrawing groups selected from the group consisting of F, Cl, Br, I, NO₂, CF₃ and a combination thereof. It is more preferred that the mixture obtained from the reaction in b) comprising the compound of formula (II) has a content, based on the weight of the mixture, of at most, preferably less than, 100 weight-ppm, preferably at most, preferably less than, 50 weight-ppm, more preferably at most, preferably less than, 10 weight-ppm, of an aryl-OH compound substituted with one or more electron-withdrawing groups.

According to the present invention, the mixture comprising a compound of formula (II) has a content, based on the total weight of the mixture of less than 100 weight-ppm, preferably less than 50 weight-ppm, more preferably less than 10 weight-ppm, even more preferably less than 5 weight-ppm of an aryl-OH that is 4-nitrophenol.

According to the present invention, the mixture comprising a compound of formula (II) has a content, based on the total weight of the mixture of less than 100 weight-ppm, preferably less than 50 weight-ppm, more preferably less than 10 weight-ppm, even more preferably less than 5 weight-ppm of an aryl-OH that is penta-fluorophenol.

The present invention is advantageous with respect to the prior art processes for example disclosed in international patent applications WO2011/123672 and WO 2014/047117 where for preparing the compound of formula (I) and in particular sofosbuvir of formula (I-A), aryloxide leaving groups are used substituted with one or more electron withdrawing groups which are considered toxic by the Food and Drug administration if present above a certain threshold in a product. The present invention provides a simple and effective synthesis which does not necessarily entail the use of aryloxide leaving groups substituted one or more elec-tron withdrawing groups. In fact, the present invention preferably provides non-toxic leaving groups (Y—)ₙR₁ wherein R₁ is selected from the groups consisting of a residue of formulas (A) to (D) and (A1) as defined above.

Preferably, the composition comprising the compound of formula (I) has a content of one or more Lewis acids comprising a twice positively charged ion or three times positively charged ion, preferably a twice positively charged ion, more preferably the Zn ion, of at most, preferably less than 1350 weight-ppm, based on the total weight of the composition and calculated based on the weight of the twice positively charged ion or three times positively charged ion, preferably the twice positively charged ion, more preferably the Zn ion, comprised in the one or more Lewis acids, wherein, in case the composition comprises more than one Lewis acid, said weight-ppm values relate to each individual Lewis acid.

Preferably, the composition is obtainable or obtained according to the process of the invention as disclosed above. Preferably, the composition is obtainable or obtained according to the process of the invention wherein in compound (II) n is 1 and the leaving group is Y—R₁ wherein R₁ is a residue selected from the group consisting of a residue of formula (A), a residue of formula (B), a residue of formula (C) and residue of formula (D) as defined above. It is also preferred that in the process in compound (II) n is 0 and the leaving group is Y—R₁ wherein R₁ is the residue of formula (A1) as defined above. It is also preferred that in compound (II) n is 1 and the leaving group is Y—R₁ wherein R₁ is the residue of formula (IIc). It is more preferred that in the process in compound (II) n is 1 and that the leaving group is O—R₁ wherein R₁ is the residue of formula (IIc). It is further more preferred that n is 1 and that the leaving group is Y—R₁ wherein R₁ is the residue of formula (IIb). It is further even more preferred that n is 1 and that the leaving group is O—R₁ wherein R₁ is the residue of formula (IIc).

### Composition Comprising a Compound of Formula (II)

Further, the present invention provides a composition of which at least 99.90 weight-%, preferably at least 99.92 weight-%, based on the weight of the composition, consist of the compound of formula (II)

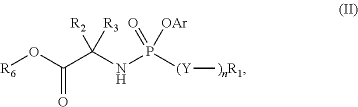

preferably comprising a compound of formula (II′)

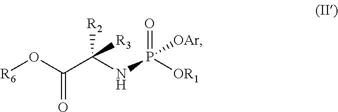

more preferably comprising a compound of formula (II-A)

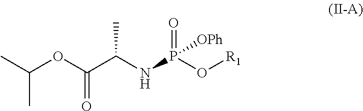

or a mixture comprising a compound of formula (II-B)

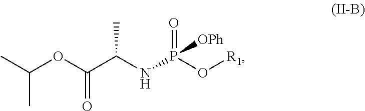

wherein R₁, R₂, R₃, R₆ and Ar are as defined, and wherein R₁ is preferably a residue selected from the group of consisting of a residue of formula (A), of formula (IIb)

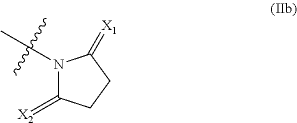

or of formula (IIc)
or

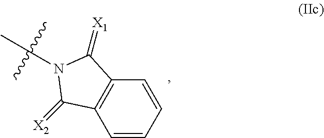

wherein X₁ is O and X₂ is O.

In the composition, compounds (II), (II′), (II-A) (II-B) are also compounds wherein R₁, R₂, R₃, R₆ and Ar are as defined above, and wherein R₁ is preferably a residue selected from the group of consisting of a residue of formula (A) as defined above in connection with compound (II′-1).

In the composition, compounds (II), (II′), (II-A) (II-B) are also compounds wherein R₁, R₂, R₃, R₆ and Ar are as defined above, and wherein R₁ is preferably a residue selected from the group of consisting of a residue of formula (B) as defined above in connection with compound (II′-1).

In the composition, compounds (II), (II′), (II-A) and (II-B) are also compounds wherein R₁, R₂, R₃, R₆ and Ar are as defined above, and wherein R₁ is preferably a residue selected from the group of consisting of a residue of formula (C) as defined above in connection with compound (II′-1).

In the composition, compounds (II) (II′), (II-A) and (II-B) are also compounds wherein R₁, R₂, R₃, R₆ and Ar are as defined above, and wherein R₁ is preferably a residue selected from the group of consisting of a residue of formula (D) as defined above in connection with compound (II′-1).

In the composition, compounds (II) is also a compound wherein R₁, R₂, R₃, R₆ and Ar are as defined above, and wherein R₁ is preferably a residue selected from the group of consisting of a residue of formula (A1) as defined above in connection with compound (II′-1).

### General Use

Generally, the present invention also relates to the use of a Lewis acid, preferably of a Lewis acid comprising a twice positively charged ion or a three times positively charged ion, more preferably of a Lewis acid comprising a twice positively charged metal ion or a three times positively charged metal ion, for the preparation of a compound of formula (I),

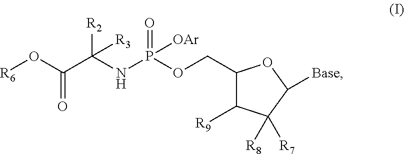

preferably for a compound of formula (I-A)

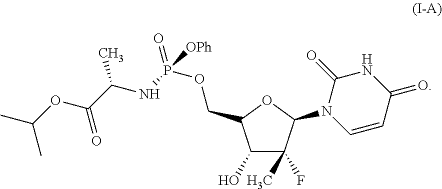

Preferably, the present invention relates to the above-defined use, wherein the twice positively charged ion is a Zn ion, a Mg ion, a Cu ion, or an Fe ion, preferably a Zn ion, and wherein the three times positively charged ion is a Mn ion.

More preferably, according to the above-defined use, the Lewis acid is one or more of one or more of ZnBr₂, ZnCl₂, ZnI₂, MgBr₂, MgBr₂.OEt₂, CuCl₂, Cu(acetylacetonate)₂, Fe(II) fumarate, Mn(acetylacetonate)₃, preferably one or more of ZnBr₂, ZnCl₂, ZnI₂, more preferably ZnBr₂.

Therefore, the present invention preferably relates to the use of ZnBr₂ for the preparation of a compound of formula (I-A)

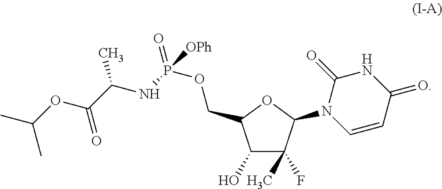

Still further, the present invention relates to the use of the compound of formula (I),

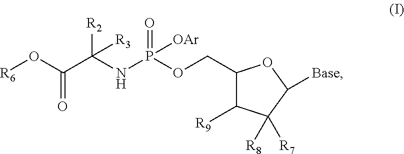

or the composition comprising the compound of formula (I) as defined above, or the mixture comprising the compound of formula (I) above, wherein the compound of formula (I) is preferably the compound of formula (I-A)

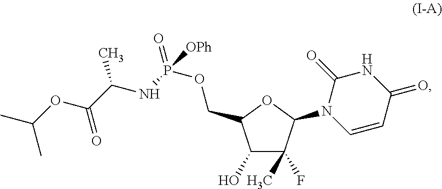

for the preparation of a pharmaceutical composition.

Yet further, the present invention relates to a pharmaceutical composition, comprising the compound of formula (I)

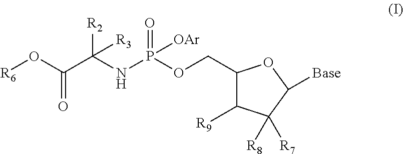

preferably the compound of the formula (I-A),

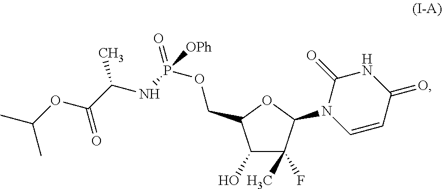

obtainable or obtained by a process according to the present invention, preferably comprising c), and at least one pharmaceutically acceptable excipient.

Yet further, the present invention relates to said pharmaceutical composition for use in a method for treating hepatitis C in a human.

The present invention is further illustrated by the following embodiments and combinations of embodiments as given by the respective dependencies and references.
1. A process for the preparation of a compound of formula (I) including all isomers, stereoisomers, enantiomers and diastereomers thereof

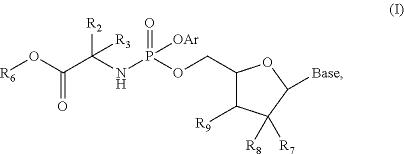

and salts thereof, the process comprising
a) providing a mixture comprising a compound of formula (II)

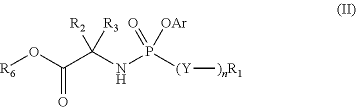

and a compound of formula (III)

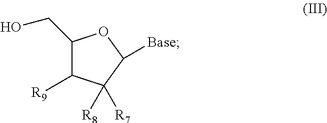

b) subjecting the mixture provided in a) to reaction conditions in the presence of one or more Lewis acids to the mixture, obtaining a mixture comprising the compound of formula (I).
2. The process of embodiment 1, wherein at each occurrence
   Ar is phenyl, naphthyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
   (Y—)ₙR₁ is a leaving group for nucleophilic substitution reaction, wherein n is 0 or 1 and wherein Y is O, N or S;
   R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, COOH, CHO, C(O)(C₁-C₆ alkyl), C(O)(aryl), COO(C₁-C₆ alkyl), COONH₂, COONH (C₁-C₆ alkyl) and CN;
   R₆ is C₁-C₆ alkyl or C₃-C₁₀ cycloalkyl optionally substituted with at least one of C₁-C₆ alkyl and aryl;
   Base is a purinyl residue or a pyrimidinyl residue linked to the furanose ring according to formula (III) through a carbon or nitrogen atom;
   R₇ and R₈ are independently H, OH, F, Cl, Br, I, azide, nitrile, NH₂, NHR₂₆, NR₂₆R₂₄, (CO)—NH₂, (CO)—NHR₂₆, (CO)—NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl;
   R₉ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl.
3. The process of embodiment 1 or 2, wherein n is 1 and R₁ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups.
4. The process of embodiment 3, wherein n is 1 and R₁ is phenyl substituted with one or more electron-withdrawing groups, wherein the one or more electron-withdrawing groups are preferably F, Cl, Br, I, or NO₂.
5. The process of embodiment 1 or 2, wherein n is 1 and R₁ is a residue of formula (A)

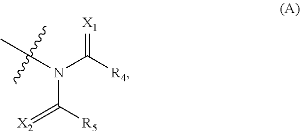

a residue of formula (B)

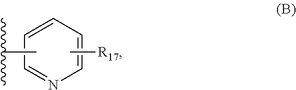

a residue of formula (C)

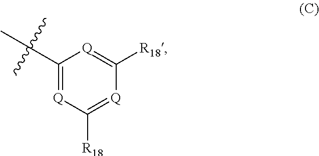

or a residue of formula (D)

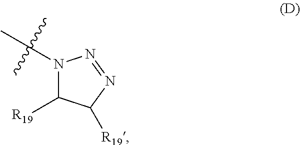

wherein at each occurrence
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R₁₈ are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.
6. The process of embodiment 1 or 2, wherein n is 0 and R₁ is a residue of formula (A1)

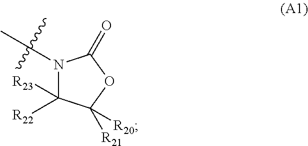

R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
7. The process of embodiment 2 or 6, wherein the substituent of the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
8. The process of any of embodiment 2, 6, 7, wherein the aromatic ring is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
9. The process of embodiment 2 or 6, wherein R₂₂ and R₂₃ are each independently H, aryl, or C₁-C₆ alkyl substituted with at least one of C₁-C₆ alkoxy optionally substituted with at least one of OH and NH₂.
10. The process of any of embodiments 1, 2, 5, wherein n is 1 and R₁ is a residue of formula (A)

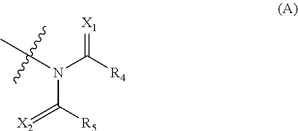

wherein
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.
11. The process of any of embodiments 1, 2, 5, 10, wherein R₁ is a residue of formula (IIb)

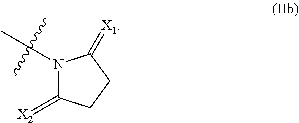

12. The process of any of embodiments 1, 2, 5, 10, wherein R₁ is a residue of formula (IIc)

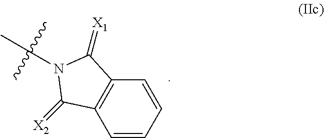

13. The process of any of embodiments 1, 2, 5, 10 to 12, wherein X₁ is O and X₂ is O.
14. The process of any embodiments 1, 2, 5, 10 to 13, wherein the Lewis acid is selected from the group consisting of ZnBr₂, ZnCl₂, ZnI₂, MgBr₂, MgBr₂.OEt₂, CuCl₂, Cu(acetylacetonate)₂, and Fe(II) fumarate, preferably ZnBr₂, ZnCl₂, ZnI₂.
15. The process of any of embodiments 1, 2, 5, wherein n is 1 and R₁ is a residue of formula (B)

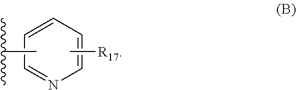

16. The process of any of embodiments 1, 2, 5, 15, wherein R₁₇ is selected from the group consisting of F, Cl, Br, I, NO₂, CHO, COOH, COO—(C₁-C₆)alkyl, CN and COCl.
17. The process of any of embodiments 1, 2, 5, wherein n is 1 and R₁ a residue of formula (C)

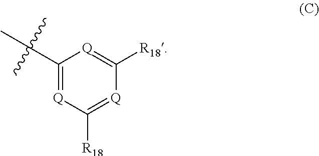

18. The process of any of embodiments 1, 2, 5, 17, wherein R₁₈ and R_{18′} are independently F, Cl, Br, I, or C₁-C₆ alkoxy and each Q is independently C or N, wherein at least one Q is N.
19. The process of any of embodiments 1, 2, 5, wherein n is 1 and R₁ or a residue of formula (D)

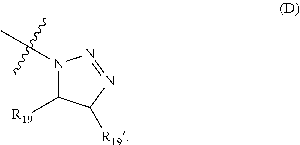

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the aromatic ring is preferably benzo,
wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.
20. The process of any of embodiments 1, 2, 5, 19, wherein the substituent of the optionally substituted 5-, 6-, or 7-membered saturated or partially unsaturated or aromatic ring is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
21. The process of any of embodiments 1, 2, 5, 19, 20, wherein the aromatic ring formed by R₁₉ and R_{19′} taken together is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
22. The process of any of embodiments 1 to 21, wherein the compound of formula (II) is the compound of formula (II-A)

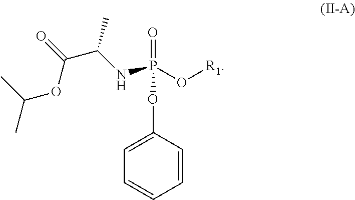

23. The process of any of embodiments 1 to 22, wherein the compound of formula (III) is the compound of formula (III-A)

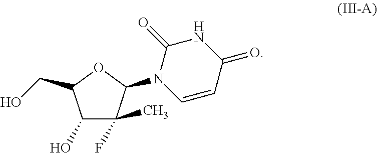

24. The process of any of embodiments 1 to 23, wherein the compound of formula (I) is the compound of formula (I-A)

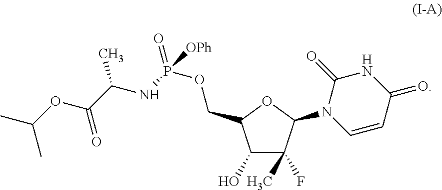

25. The process of any of embodiments 1 to 21, preferably 1 to 13, wherein the compound of formula (II) is the compound of formula (II-A)

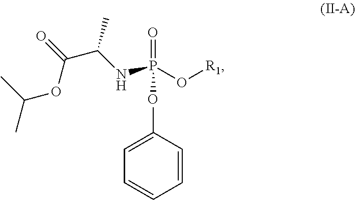

the compound of formula (III) is the compound of formula (III-A)

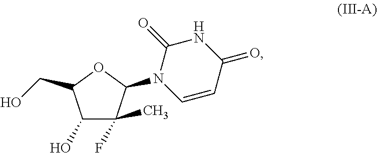

and the compound of formula (I) is the compound of formula (I-A)

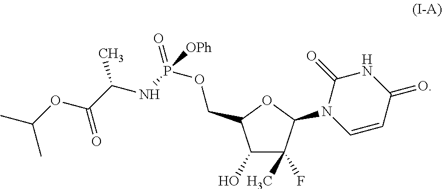

26. The process of any of embodiments 1 to 25, wherein the one or more Lewis acids according to b) comprise a twice positively charged ion or a three times positively charged ion.
27. The process of any of embodiments 1 to 26, wherein the one or more Lewis acids according to b) comprise a twice positively charged metal ion or a three times positively charged metal ion.
28. The process of embodiment 26 or 27, wherein the twice positively charged ion is a Zn ion, a Mg ion, a Cu ion, or an Fe ion.
29. The process of any of embodiments 26 to 28, wherein the twice positively charged ion is a Zn ion.
30. The process of any of embodiments 1 to 29, preferably of embodiments 10 to 13, wherein the one or more Lewis acids is one or more of ZnBr₂, ZnCl₂, ZnI₂.
31. The process of any of embodiments 26 to 30, wherein the one or more Lewis acids comprises, preferably is ZnBr₂.
32. The process of embodiments 28, wherein the one or more Lewis acids is one or more of ZnBr₂, ZnCl₂, ZnI₂, MgBr₂, MgBr₂.OEt₂, CuCl₂, Cu(acetylacetonate)₂, and Fe(II) fumarate.
33. The process of embodiment 26 or 27, wherein the three times positively charged ion is a Mn ion.
34. The process of embodiment 33, wherein the one or more Lewis acids is Mn(acetylacetonate)₃.
35. The process of any of embodiments 1 to 34, wherein prior to the reaction according to b), the molar ratio of the compound of formula (II) relative to the compound of formula (III) is in the range of from 0.5:1 to 5:1.
36. The process of embodiment 35, wherein the molar ratio of the compound of formula (II) relative to the compound of formula (III) is in the range of from 0.8:1 to 2:1, preferably in the range of from 0.9:1 to 1.2:1.
37. The process of any of embodiments 1 to 36, wherein prior to the reaction according to b), the molar ratio of the Lewis acid relative to the compound of formula (III) is in the range of from 0.1:1 to 5:1.
38. The process of embodiment 37, wherein the molar ratio of the Lewis acid relative to the compound of formula (III) is in the range of from 0.2:1 to 2:1, preferably in the range of from 0.5:1 to 1.2:1.
39. The process of any of embodiments 1 to 38, wherein the mixture provided in a) further comprises one or more solvents.
40. The process of embodiment 39, wherein the one or more solvents are one or more organic solvents.
41. The process of embodiment 40, wherein the one or more organic solvents are aprotic organic solvents.
42. The process of any of embodiments 39 to 41, wherein the one or more solvents are selected from the group consisting of methylene chloride, methyl tert-butyl ether, tetrahydrofurane, dimethylsulphoxide, dimethylformamide, and a mixture of two or more thereof.
43. The process of any of embodiments 1 to 42, wherein the mixture provided in a) further comprises one or more bases.
44. The process of embodiment 43, wherein the one or more bases are organic bases.
45. The process of embodiment 43 or 44, wherein the one or more bases are selected from the group consisting of an amine, an amidine, a heteroaromatic compound comprising a basic ring-nitrogen atom, and a mixture of two or more thereof, more preferably selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof.
46. The process of any of embodiments 43 to 45, wherein prior to the reaction according to b), the molar ratio of the one or more bases relative to the compound of formula (III) is in the range of from 0.1:1 to 5:1 wherein, if more than one base is comprised in the mixture provided in a), the molar ratio relates to the total molar amount of all bases.
47. The process of embodiment 46, wherein the molar ratio of the one or more bases relative to the compound of formula (III) is in the range of from 0.1:1 to 2:1 preferably in the range of from 0.5:1 to 1.2:1 wherein, if more than one base is comprised in the mixture provided in a), the molar ratio relates to the total molar amount of all bases.
48. The process of any of embodiments 1 to 47, wherein the mixture provided in a) further comprises one or more solvents and one or more bases, wherein prior to the reaction according to b), the molar ratio of the one or more bases relative to the compound of formula (III) is in the range of from 0.1:1 to 5:1.
49. The process of embodiment 48, wherein the one or more solvents are selected from the group consisting of methylene chloride, methyl tert-butyl ether, tetrahydrofurane, dimethylsulphoxide, dimethylformamide, and a mixture of two or more thereof and the one or more bases are selected from the group consisting of an amine, an amidine, a heteroaromatic compound comprising a basic ring-nitrogen atom, and a mixture of two or more thereof, more preferably selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof.
50. The process of embodiment 49, wherein prior to the reaction according to b), the molar ratio of the compound of formula (II) relative to the compound of formula (III) is in the range of from 0.9:1 to 1.2:1, the molar ratio of the Lewis acid relative to the compound of formula (III) is in the range of from 0.5:1 to 1.2:1, and the molar ratio of the one or more bases relative to the compound of formula (III) is in the range of from 0.5:1 to 1.2:1 wherein, if more than one base is comprised in the mixture provided in a), the molar ratio relates to the total molar amount of all bases.
51. The process of embodiment 50, wherein the reaction conditions according to b) comprise a temperature of the mixture in the range of from 0 to 80° C.
52. The process of embodiment 51, wherein the temperature is in the range of from 10 to 65° C.
53. The process of embodiment 52, wherein the temperature is in the range of from 20 to 50° C.
54. The process of any of embodiments 1 to 53, wherein according to b), the mixture is subjected to the reaction conditions for a period of time in the range of from 0.5 to 48 h.
55. The process of embodiment 54, wherein the period of time is in the range of from 1 to 36 h.
56. The process of embodiment 55, wherein the period of time is in the range of from 2 to 24 h.
57. The process of any of embodiments 1 to 56, wherein the reaction conditions according to b) comprise a temperature of the mixture in the range of from 20 to 50° C., wherein according to b), the mixture is subjected to the reaction conditions for a period of time in the range of from 2 to 24 h.
58. The process of any of embodiments 1 to 57, wherein the mixture provided in a) further comprises one or more solvents and one or more bases, wherein prior to the reaction according to b), the molar ratio of the one or more bases relative to the compound of formula (III) is in the range of from 0.1:1 to 5:1.
59. The process of embodiment 58, wherein the one or more solvents are selected from the group consisting of methylene chloride, methyl tert-butyl ether, tetrahydrofurane, dimethylsulphoxide, dimethylformamide, and a mixture of two or more thereof and the one or more bases are selected from the group consisting of an amine, an amidine, a heteroaromatic compound comprising a basic ring-nitrogen atom, and a mixture of two or more thereof, more preferably selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof.
60. The process of embodiment 59, wherein prior to the reaction according to b), the molar ratio of the compound of formula (II) relative to the compound of formula (III) is in the range of from 0.9:1 to 1.2:1, the molar ratio of the Lewis acid relative to the compound of formula (III) is in the range of from 0.5:1 to 1.2:1, and the molar ratio of the one or more bases relative to the compound of formula (III) is in the range of from 0.5:1 to 1.2:1 wherein, if more than one base is comprised in the mixture provided in a), the molar ratio relates to the total molar amount of all bases.
61. The process of embodiment 60, wherein the reaction conditions according to b) comprise a temperature of the mixture in the range of from 20 to 50° C., wherein according to b), the mixture is subjected to the reaction conditions for a period of time in the range of from 2 to 24 h.
62. The process of any of embodiments 1 to 61, wherein the mixture obtained in b) comprising the compound of formula (I) has a content, based on the weight of the mixture, of at most, preferably less than 100 weight-ppm of an aryl-OH compound substituted with one or more electron-withdrawing groups.
63. The process of embodiment 62, wherein the one or more electron-withdrawing groups are selected from the group consisting of F, Cl, Br, I, NO₂, CF₃ and a combination thereof.
64. The process of embodiment 62 or 63, wherein the aryl-OH compound is selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol.
65. The process of any of embodiments 62 to 64, wherein the content is at most, preferably less than 50 weight-ppm.
66. The process of embodiment 65, wherein the content is at most, preferably less than 10 weight-ppm.
67. The process of any of embodiments 1 to 66, further comprising
   c) separating the compound of formula (I) from the mixture obtained in step b).
68. The process of claim 67, wherein the separating in c) comprises
   c1) crystallizing the compound of formula (I) in the mixture obtained in b), obtaining the crystallized compound of formula (I) in its mother liquor;
   c2) separating the compound of formula (I) from its mother liquor.
69. The process of embodiment 68, wherein the crystallizing according to c1) comprises seeding.
70. The process of any of embodiments 67 to 69, wherein the separating according to c) or the separating according to c2) comprises filtration, centrifugation, drying, or a mixture of two or more thereof.
71. The process of embodiment 70, wherein drying comprises drying in an atmosphere comprising oxygen, nitrogen, or a mixture thereof.
72. The process of embodiment 70 or 71, wherein drying comprises rapid-drying, preferably spray-drying.
73. The process of any of embodiments 68 to 72, wherein the separating according to c) or the separating according to c2) comprises filtration, drying, and optionally washing.
74. The process of embodiment 67, wherein the separating in c) comprises
   c1) crystallizing the compound of formula (I) in the mixture obtained in b), obtaining the crystallized compound of formula (I) in its mother liquor, the crystallizing optionally comprising seeding;
   c2) separating the compound of formula (I) from its mother liquor, comprising
      c21) subjecting the mother liquor comprising the crystallized compound of formula (I) to filtration;
      c22) optionally washing the filter cake;
      c23) drying the optionally washed filter cake.
75. The process of any of embodiments 67 to 74, wherein the composition obtained from c) or c2) has a content of the one or more Lewis acids comprising a twice positively charged ion or three times positively charged ion, preferably a twice positively charged ion, more preferably the Zn ion, of at most, preferably less than 1350 weight-ppm, based on the total weight of the composition and calculated based on the weight of the twice positively charged ion or three times positively charged ion, preferably the twice positively charged ion, more preferably the Zn ion, comprised in the one or more Lewis acids, wherein, in case the composition comprises more than one Lewis acid, said weight-ppm values relate to each individual Lewis acid.
76. The process of embodiment 75, wherein the content is at most, preferably less than 600 weight-ppm.
77. The process of embodiment 76, wherein the content is at most, preferably less than 100 weight-ppm.
78. The process of any of embodiments 1 to 77, further comprising providing the mixture according to a) by a process comprising
   i) providing a mixture comprising a compound of formula (IV)

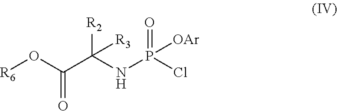

and a compound R₁(—Y)ₙH;
ii) subjecting the mixture provided in i) to reaction conditions, obtaining a mixture comprising the compound of formula (II).
79. A process for the preparation a compound of formula (II)

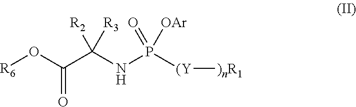

wherein
Ar is phenyl, naphthyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
(Y—)ₙR₁ is a leaving group for nucleophilic substitution reaction, wherein n is 0 or 1 and wherein Y is O, N or S;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, COOH, CHO, C(O)(C₁-C₆ alkyl), C(O)(aryl), COO(C₁-C₆ alkyl), COONH₂, COONH (C₁-C₆ alkyl) and CN;
R₆ is C₁-C₆ alkyl or C₃-C₁₀ cycloalkyl optionally substituted with at least one of C₁-C₆ alkyl and aryl;
the process comprising
i) providing a mixture comprising a compound of formula (IV)

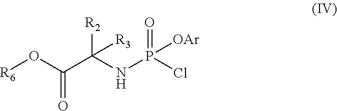

and a compound R₁(—Y)ₙH;
ii) subjecting the mixture provided in i) to reaction conditions, obtaining a mixture comprising the compound of formula (II).
80. The process of embodiment 79, wherein n is 1 and R₁ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups, more preferably phenyl substituted with one or more electron-withdrawing groups, wherein the one or more electron-withdrawing groups are preferably F, Cl, Br, I, or NO₂.
81. The process of embodiment 79, wherein n is 1 and R₁ is a residue of formula (A)

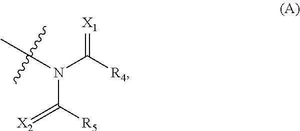

a residue of formula (B)

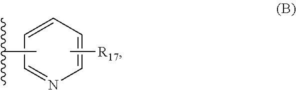

a residue of formula (C)

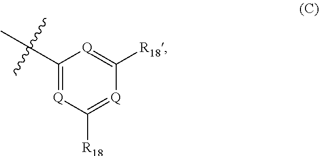

or a residue of formula (D)

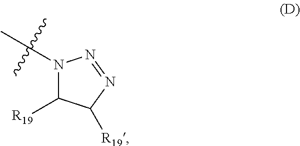

wherein at each occurrence
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl, wherein R is preferably the residue according to formula (A).
82. The process of embodiment 79, wherein n is 0 and R₁ is a residue of formula (A1)

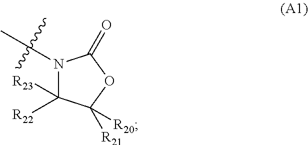

wherein
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
83. The process of embodiment 81 or 82, wherein the substituent of the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
84. The process of any of embodiments 81 to 83, wherein the aromatic ring is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
85. The process of embodiment 81 or 82, wherein R₂₂ and R₂₃ are each independently H, aryl, or C₁-C₆ alkyl substituted with at least one of C₁-C₆ alkoxy optionally substituted with at least one of OH and NH₂.
86. The process of embodiment 79 or 81, wherein n is 1 and R₁ is a residue of formula (A)

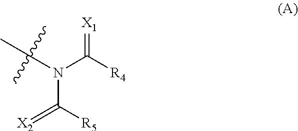

wherein
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.
87. The process of embodiment 81 or 86, wherein R₁ is a residue of formula (IIb)

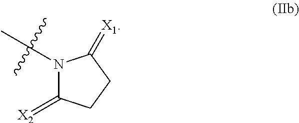

88. The process of embodiment 81 or 86, wherein R₁ is a residue of formula (IIc)

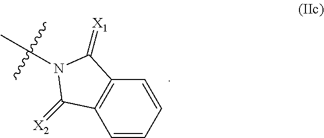

89. The process of any of embodiments 81, 86 to 88, wherein X₁ is O and X₂ is O.
90. The process of any of embodiments 81, 86 to 88, wherein X₁ is O and X₂ is S.
91. The process of embodiment 79 or 81, wherein n is 1 and R₁ is a residue of formula (B)

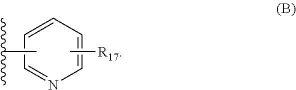

92. The process of embodiment 81 or 91, wherein R₁₇ is selected from the group consisting of F, Cl, Br, I, NO₂, CHO, COOH, COO—(C₁-C₆)alkyl, CN and COCl.
93. The process of embodiment 79 or 81, wherein n is 1 and R₁ a residue of formula (C)

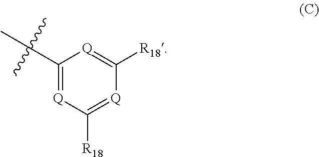

94. The process of embodiment 81 or 93, wherein R₁₈ and R_{18′} are independently F, Cl, Br, I, or C₁-C₆ alkoxy and each Q is independently C or N, wherein at least one Q is N.
95. The process of embodiment 79 or 81, wherein n is 1 and R₁ or a residue of formula (D)

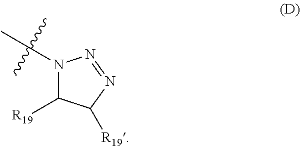

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the aromatic ring is preferably benzo,
wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl,
96. The process of embodiment 81 or 95, wherein the substituent of the optionally substituted 5-, 6-, or 7-membered saturated or partially unsaturated or aromatic ring is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
97. The process of any of embodiments 81, 95, 96, wherein the aromatic ring formed by R₁₉ and R_{19′} taken together is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
98. The process of any of embodiments 78 and 79 to 97, wherein in the mixture provided in (i), the molar ratio of the compound of formula (IV) relative to the compound R₁(—Y)ₙH is in the range of from 0.5:1 to 2:1.
99. The process of embodiment 98, wherein the molar ratio of the compound of formula (IV) relative to the compound R₁(—Y)ₙH is in the range of from 0.7:1 to 1.3:1, preferably in the range of from 0.9:1 to 1.1:1.
100. The process of any of embodiments 78 and 79 to 99, wherein the mixture provided in i) further comprises one or more solvents.
101. The process of embodiment 100, wherein the one or more solvents are one or more organic solvents.
102. The process of embodiment 100 or 101, wherein the one or more organic solvents are one or more aprotic organic solvents.
103. The process of any of embodiments 100 to 102, wherein the one or more solvents are selected from the group consisting of methylene chloride, methyl tert-butyl ether, tetrahydrofurane, dimethylsulphoxide, dimethylformamide and a mixture of two or more thereof.
104. The process of any of embodiments 78 and 79 to 103, wherein the mixture provided in i) further comprises one or more bases.
105. The process of embodiment 104, wherein the one or more bases are one or more organic bases.
106. The process of embodiment 104 or 105, wherein the one or more bases are selected from the group consisting of an amine, an amidine, a heteroaromatic compound comprising a basic ring-nitrogen atom, and a mixture of two or more thereof, preferably selected from the group consisting of ethyldiisopropylamine, triethylamine, diethylamine, 1,8 diazabicycloundec-7-ene, pyridine, quinoline, isoquinoline, acridine, pyrazine, imidazole, benzimidazole, pyrazole, and a mixture of two or more thereof.
107. The process of any of embodiments 104 to 106, wherein in the mixture provided in i), the molar ratio of the one or more bases relative to the compound R₁(—Y)ₙH is in the range of from 0.05:1 to 5:1, wherein, if more than one base is comprised in the mixture provided in i), the molar ratio relates to the total molar amount of all bases.
108. The process of embodiment 107, wherein the molar ratio of the one or more bases relative to the compound R₁(—Y)ₙH is in the range of from 0.1:1 to 2:1, preferably in the range of from 0.5:1 to 1.2:1, wherein, if more than one base is comprised in the mixture provided in i), the molar ratio relates to the total molar amount of all bases.
109. The process of any of embodiments 78 and 79 to 108, wherein the reaction conditions according to ii) comprise a temperature of the mixture in the range of from 0 to 30° C.
110. The process of embodiment 109, wherein the temperature is in the range of from 0 to 20° C.
111. The process of embodiment 110, wherein the temperature is in the range of from 0 to 10° C.
112. The process of any of embodiments 78 and 79 to 111, wherein according to ii), the mixture is subjected to the reaction conditions for a period of time in the range of from 0.5 to 48 h.
113. The process of embodiment 112, wherein the period of time is in the range of from 1 to 36 h.
114. The process of embodiment 112 or 113, wherein the period of time is in the range of from 2 to 24 h.
115. The process of any of embodiments 78 and 79 to 114, wherein the reaction conditions according to ii) comprise a temperature of the mixture in the range of from 0 to 10° C., wherein according to ii), the mixture is subjected to the reaction conditions for a period of time in the range of from 2 to 24 h.
116. The process of any of embodiments 78 and 79 to 115, further comprising
   iii) separating the compound of formula (II) from the mixture obtained from ii).
117. The process of embodiment 116, wherein the separating according to (iii) comprises iii1) crystallizing the compound of formula (II) in the mixture obtained in step (ii), obtaining the crystallized compound of formula (II) in its mother liquor; and
   iii2) separating the compound of formula (II) from its mother liquor, preferably by filtration.
118. The process of embodiment 117, wherein the crystallizing according to iii1) comprises seeding.
119. The process of embodiment 116 or 117, wherein the separating according to iii) or the separating according to iii2) comprises filtration, centrifugation, drying, or a mixture of two or more thereof.
120. The process of embodiment 119, wherein drying comprises drying in an atmosphere comprising oxygen, nitrogen, or a mixture thereof.
121. The process of any of embodiments 116 to 120, wherein the separating according to iii) or the separating according to iii2) comprises filtration, drying, and optionally washing.
122. The process of embodiment 116, wherein the separating in iii) comprises
   iii1) crystallizing the compound of formula (II) in the mixture obtained in ii), obtaining the crystallized compound of formula (II) in its mother liquor, the crystallizing optionally comprising seeding;
   iii2) separating the compound of formula (II) from its mother liquor, comprising
      iii21) subjecting the mother liquor comprising the crystallized compound of formula (II) to filtration;
      iii22) optionally washing the filter cake;
      iii23) drying the optionally washed filter cake.
123. The process of embodiment 122, wherein the separating in iii) comprises
   iii0) subjecting the mixture obtained in ii) to extraction, preferably comprising
   iii01) extraction, preferably with water, obtaining an organic phase comprising the compound of formula (II);
   iii02) removing the solvent from the organic phase obtained in iii01);
   iii03) dissolving the solid obtained in iii02 in one or more solvents;
   iii1) crystallizing the compound of formula (II) in the mixture obtained in iii03), obtaining the crystallized compound of formula (II) in its mother liquor, the crystallizing optionally comprising seeding;
   iii2) separating the compound of formula (II) from its mother liquor, comprising
      iii21) subjecting the mother liquor comprising the crystallized compound of formula (II) to filtration;
      iii22) optionally washing the filter cake;
      iii23) drying the optionally washed filter cake.
124. The process of embodiment 123, wherein the one or more solvents according to iii03) comprise methyl tert-butyl ether.
125. The process of any of embodiments 117 to 123, wherein the crystallization in iii1) comprises at least two sequences of dissolving with subsequent crystallizing.
126. A mixture comprising a compound of formula (I) including all isomers, stereoisomers, enantiomers and diastereomers thereof

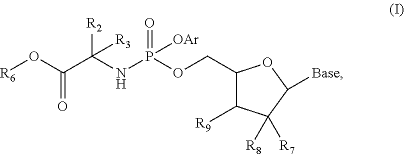

and salts thereof, obtainable or obtained by a process according to any of embodiments 1 to 78 and of embodiments 98 to 125 insofar as embodiments 98 to 125 are dependent on embodiment 78.
127. A mixture comprising a compound of formula (II)

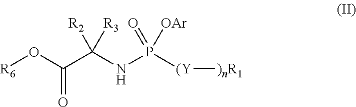

obtainable or obtained by a process according to any of embodiments 78 to 97 and 98 to 125 insofar as embodiments 98 to 125 are dependent on embodiment 78.
128. The mixture of embodiment 127, wherein
   (Y—)ₙR₁ is a leaving group for nucleophilic substitution reaction, wherein n is 0 or 1 and
   wherein Y is O, N or S;
   wherein, when n is 1,
   R₁ is a residue of formula (A)

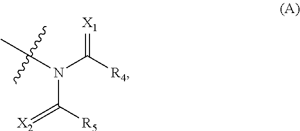

a residue of formula (B)

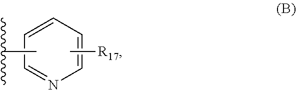

a residue of formula (C)

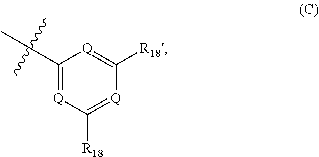

or a residue of formula (D)

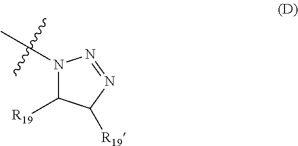

or wherein, when n is 0,
R₁ is a residue of formula (A1)

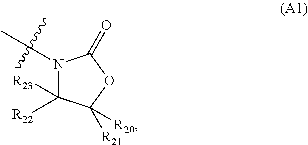

wherein at each occurrence
X₁ and X₂ are independently O or S;
R₄ and R₅ are independently H, OH, NH₂, C₁-C₆ alkyl or C₁-C₆ alkoxy, or R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
129. The mixture of embodiment 127 or 128, wherein n is 0 and R₁ is a residue of formula (A1)

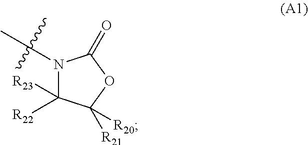

R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
130. The mixture of embodiment 128 or 129, wherein the substituent the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
131. The mixture of any of embodiments 128 to 130, wherein the aromatic ring is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
132. The mixture of embodiment 128 or 129, wherein R₂₂ and R₂₃ are each independently H, aryl, or C₁-C₆ alkyl substituted with at least one of C₁-C₆ alkoxy optionally substituted with at least one of OH and NH₂.
133. The mixture of embodiment 127 or 128, wherein n is 1 and R₁ is a residue of formula (A)

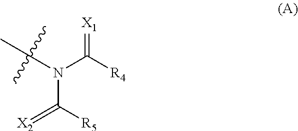

wherein
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.
134. The mixture of any of embodiments 127, 128, 133, wherein R₁ is a residue of formula (IIb)

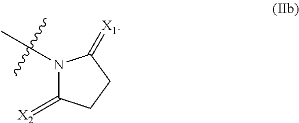

135. The mixture of any of embodiments 127, 128, 133, wherein R₁ is a residue of formula (IIc)

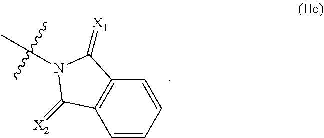

136. The mixture of any of embodiments 127, 128, 133 to 135, wherein X₁ is O and X₂ is O.
137. The mixture of any embodiments 127, 128, 133 to 135, wherein X₁ is S and X₂ is S.
138. The mixture of embodiment 127 or 128, wherein n is 1 and R₁ is a residue of formula (B)

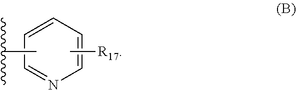

139. The mixture of embodiment 128 or 138, wherein R₁₇ is selected from the group consisting of F, Cl, Br, I, NO₂, CHO, COOH, COO—(C₁-C₆)alkyl, CN and COCl.
140. The mixture of embodiment 127 or 128, wherein n is 1 and R₁ a residue of formula (C)

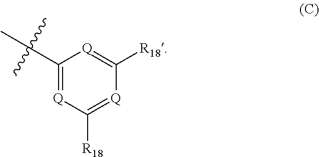

141. The mixture of embodiment 128 or 140, wherein R₁₈ and R_{18′} are independently F, Cl, Br, I, or C₁-C₆ alkoxy and each Q is independently C or N, wherein at least one Q is N.
142. The mixture of embodiment 127 or 128, wherein n is 1 and R₁ or a residue of formula (D)

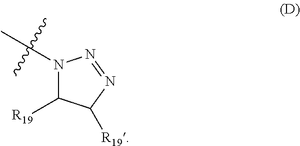

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the aromatic ring is preferably benzo,
wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.
143. The mixture of embodiment 128 or 142, wherein the substituent of the optionally substituted 5-, 6-, or 7-membered saturated or partially unsaturated or aromatic ring is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
144. The mixture of any of embodiments 128, 142, 143, wherein the aromatic ring formed by R₁₉ and R_{19′} taken together is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
145. The mixture of any of embodiments 126 to 144, having a content, based on the weight of the mixture, of at most, preferably less than 100 weight-ppm, of an aryl-OH compound substituted with one or more electron-withdrawing groups.
146. The mixture of embodiment 145, wherein the one or more electron-withdrawing groups are selected from the group consisting of F, Cl, Br, I, NO₂, CF₃ and a combination thereof.
147. The mixture of embodiment 145 or 146, wherein the aryl-OH compound is selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol.
148. The mixture of any of embodiments 145 to 147, wherein the content is at most, preferably less than 50 weight-ppm.
149. The mixture of embodiment 148, wherein the content is at most, preferably less than 10 weight-ppm.
150. A composition of which at least 99.90 weight-%, based on the weight of the composition, consist of the compound of formula (I) including isomers, stereoisomers, enantiomers, diastereomers thereof

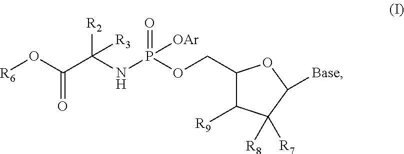

and salts thereof, preferably consist of the compound of formula (I-A)

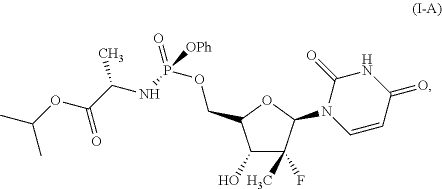

wherein
Ar is phenyl, naphtyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl) and CN;
R₆ is C₁-C₆ alkyl or C₃-C₁₀ cycloalkyl optionally substituted with at least one of C₁-C₆ alkyl and aryl;
Base is a purinyl residue or a pyrimidinyl residue linked to the furanose ring according to formula (I) through a carbon or nitrogen atom;
R₇ and R₈ are independently H, OH, halogen, azide, nitrile, NH₂, NHR₂₆, NR₂₆R₂₄, (CO)—NH₂, (CO)—NHR₂₆, (CO)—NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl;
R₉ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl
and wherein said composition has a content, based on the total weight of the composition, of less than 100 weight-ppm, preferably less than 50 weight-ppm, more preferably less than 10 weight-ppm of an aryl-OH compound substituted with one or more elec-tron-withdrawing groups wherein the one or more electron-withdrawing groups are
preferably selected from the group consisting of F, Cl, Br, I, NO₂, CF₃ and a combination thereof, wherein the aryl-OH compound is preferably selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol.
151. The composition of embodiment 150, wherein at least 99.92 weight-%, based on the weight of the composition, consist of the compound of formula (I) including isomers, stereoisomers, enantiomers, diastereomers thereof, and salts thereof.
152. The composition of embodiment 151 wherein at least 99.95 weight-%, based on the weight of the composition, consist of the compound of formula (I) including isomers, stereoisomers, enantiomers, diastereomers thereof, and salts thereof.
153. The composition of any of embodiments 150 to 152, obtainable or obtained by a process according to any of embodiments 1 to 78 and 98 to 125 insofar as embodiments 98 to 125 are dependent on embodiment 78.
154. The composition of any of embodiments 150 to 153, having a content of the one or more Lewis acids comprising a twice positively charged ion or three times positively charged ion, preferably a twice positively charged ion, more preferably the Zn ion, of at most, preferably less than 1350 weight-ppm, based on the total weight of the composition and calculated based on the weight of the twice positively charged ion or three times positively charged ion, preferably the twice positively charged ion, more preferably the Zn ion, comprised in the one or more Lewis acids, wherein, in case the composition comprises more than one Lewis acid, said weight-ppm values relate to each individual Lewis acid.
155. The composition of embodiment 154, wherein the content is at most, preferably less than 600 weight-ppm.
156. The composition of embodiment 154 or 155, wherein the content is at most, preferably less than 110 weight-ppm.
157. A composition of which at least 99.90 weight-% consist of the compound of formula (II)

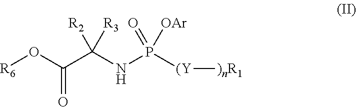

wherein
Ar is phenyl, naphtyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, COOH, CHO, C(O)(C₁-C₆ alkyl), C(O)(aryl), COO(C₁-C₆ alkyl), COONH₂, COONH C₁-C₆ alkyl) and CN;
R₆ is C₁-C₆ alkyl or C₃-C₁₀ cycloalkyl optionally substituted with at least one of C₁-C₆ alkyl and aryl;
(Y—)ₙR₁ is a leaving group for nucleophilic substitution reaction, wherein n is 0 or 1 and wherein Y is O, N or S;
wherein, when n is 1,
R₁ is a residue of formula (A)

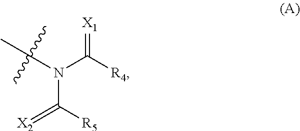

a residue of formula (B)

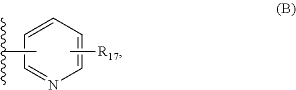

a residue of formula (C)

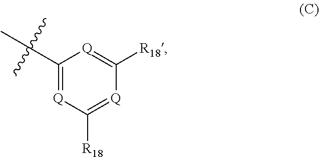

or a residue of formula (D)

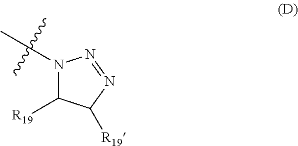

or wherein, when n is 0,
R₁ is a residue of formula (A1)

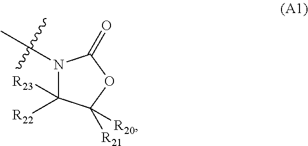

wherein at each occurrence
X₁ and X₂ are independently O or S;
R₄ and R₅ are independently H, OH, NH₂, C₁-C₆ alkyl or C₁-C₆ alkoxy, or R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl; R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl;
and wherein
said composition has a content, based on the weight of the mixture, of less than 100 weight-ppm, preferably less than 50 weight-ppm, more preferably less than 10 weight-ppm of an aryl-OH compound substituted with one or more electron-withdrawing groups wherein the one or more electron-withdrawing groups are preferably selected from the group consisting of F, Cl, Br, I, NO₂, CF₃ and a combination thereof, wherein the aryl-OH compound is preferably selected from the group consisting of 2-nitrophenol, 4-nitrophenol, 2,4-dinitro-phenol, penta-fluorophenol, 2-chloro-4-nitrophenol, 2,4-dichlorophenol, and 2,4,6-trichlorophenol.
158. The composition of embodiment 157 wherein n is 0 and R₁ is a residue of formula (A1)

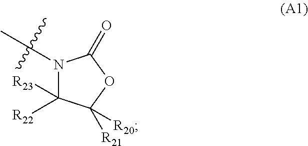

R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
159. The composition of embodiment 157 or 158, the substituent of the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
160. The composition of any of embodiments 157 to 159, wherein the aromatic ring is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
161. The composition of embodiment 157 or 158, wherein R₂₂ and R₂₃ are each independently H, aryl, or C₁-C₆ alkyl substituted with at least one of C₁-C₆ alkoxy optionally substituted with at least one of OH and NH₂.
162. The composition of embodiment 157, wherein n is 1 and R₁ is a residue of formula (A)

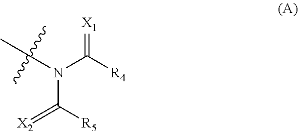

wherein
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.
163. The composition of embodiment 157 or 162, wherein R₁ is a residue of formula (IIb)

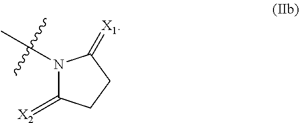

164. The composition of embodiment 157 or 162, wherein R₁ is a residue of formula (IIc)

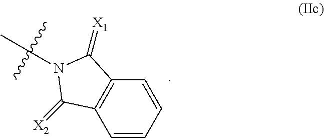

165. The composition of any of embodiments 157, 162 to 164, wherein X₁ is O and X₂ is O.
166. The composition of any embodiments 157, 162 to 164, wherein X₁ is S and X₂ is S.
167. The composition of embodiment 157, wherein n is 1 and R₁ is a residue of formula (B)

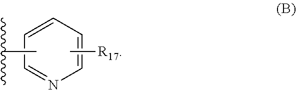

168. The composition of embodiment 157 or 167, wherein R₁₇ is selected from the group consisting of F, Cl, Br, I, NO₂, CHO, COOH, COO—(C₁-C₆)alkyl, CN and COCl.
169. The composition of embodiment 157, wherein n is 1 and R₁ a residue of formula (C)

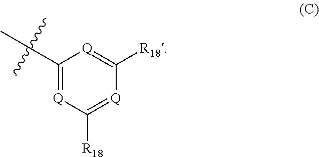

170. The composition of embodiment 157 or 169, wherein R₁₈ and R_{18′} are independently F, Cl, Br, I, or C₁-C₆ alkoxy and each Q is independently C or N, wherein at least one Q is N.
171. The composition of embodiment 157, wherein n is 1 and R₁ or a residue of formula (D)

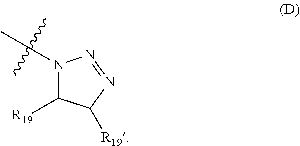

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring wherein the aromatic ring is preferably benzo,
wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl,
172. The composition of embodiment 157 or 171, wherein the substituent of the optionally substituted 5-, 6-, or 7-membered saturated or partially unsaturated or aromatic ring is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
173. The composition of any of embodiments 157, 171 or 172, wherein the aromatic ring formed by R₁₉ and R_{19′} taken together is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
174. The composition of any of embodiments 157 to 173, wherein at least 99.92 weight-% consist of the compound of formula (II).
175. The composition of any embodiments 157 to 174, wherein at least 99.95 weight-% consist of the compound of formula (II).
176. The composition of any of embodiments 157 to 175, wherein n is 1.
177. The composition of any of embodiments 157 to 176, wherein Y is O.
178. The composition of any of embodiments 157 to 177, obtainable or obtained by a process according to any of embodiments 1 to 97 and 98 to 125 insofar as embodiments 98 to 125 are dependent on embodiment 97.
179. A compound of formula (II)

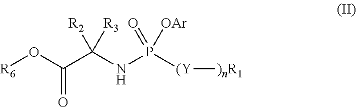

wherein
Ar is phenyl, naphtyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl) and CN;
R₆ is C₁-C₆ alkyl or C₃-C₁₀ cycloalkyl optionally substituted with at least one of C₁-C₆ alkyl and aryl;
(Y—)ₙR₁ is a leaving group for nucleophilic substitution reaction, wherein n is 0 or 1 and
wherein Y is O, N or S;
wherein, when n is 1,
R₁ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups, more preferably phenyl substituted with one or more elec-tron-withdrawing groups, wherein the one or more electron-withdrawing groups are preferably F, Cl, Br, I, or NO₂; or
R₁ is a residue of formula (A)

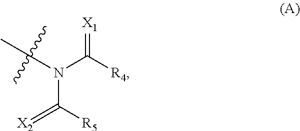

a residue of formula (B)

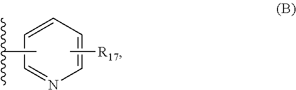

a residue of formula (C)

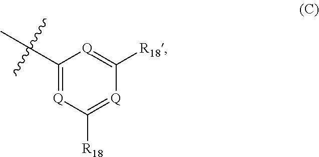

or a residue of formula (D)

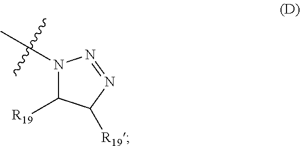

or wherein, when n is 0,
R₁ is a residue of formula (A1)

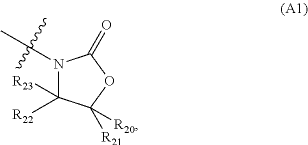

wherein at each occurrence
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
180. The compound of formula (II) of embodiment 179, wherein
   (Y—)ₙR₁ is a leaving group for nucleophilic substitution reaction, wherein n is 0 or 1 and
   wherein Y is O, N or S;
   wherein, when n is 1,
   R₁ is a residue of formula (A)

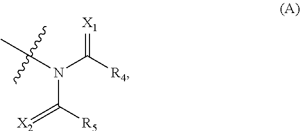

a residue of formula (B)

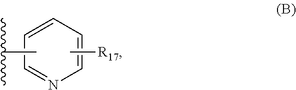

a residue of formula (C)

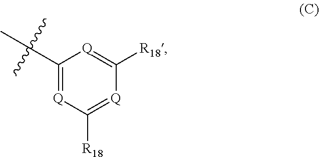

or a residue of formula (D)

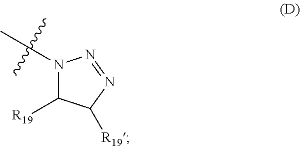

or wherein, when n is 0,
R₁ is a residue of formula (A1)

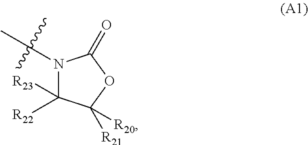

wherein at each occurrence
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group, preferably F, Cl, Br, I, NO₂, CHO, COOH, COO—(C₁-C₆)alkyl, CN, or COCl;
R₁₈ and R₁₈ are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl; R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
181. The compound of embodiment 179 and 180, wherein n is 0 and R₁ is a residue of formula (A1)

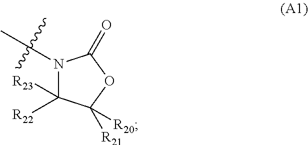

R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.
182. The compound of any of embodiments 179 to 181, wherein the substituent of the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
183 The compound of any of embodiments 179 to 182, wherein the aromatic ring is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
184. The compound of any of embodiments 179, 180, 181, wherein R₂₂ and R₂₃ are each independently H, aryl, or C₁-C₆ alkyl substituted with at least one of C₁-C₆ alkoxy optionally substituted with at least one of OH and NH₂.
185. The compound of embodiment 179 or 180, wherein n is 1 and R₁ is a residue of formula (A)

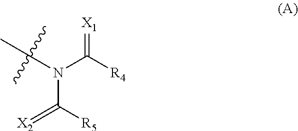

wherein
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.
186. The compound of any of embodiments 179 or 180 or 185, wherein R₁ is a residue of formula (IIb)

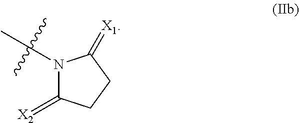

187. The compound of any of embodiments 179 or 180 or 185, wherein R₁ is a residue of formula (IIc)

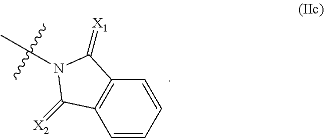

188. The compound of any of embodiments 179 or 180, 185 to 187 wherein X₁ is 0 and X₂ is O.
189. The compound of any embodiments 179, 180, 185 to 187, wherein X₁ is S and X₂ is S.
190. The compound of embodiment 179 or 180, wherein n is 1 and R₁ is a residue of formula (B)

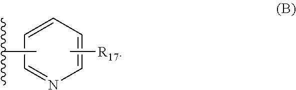

191. The compound of embodiment 190, wherein R₁₇ is selected from the group consisting of F, Cl, Br, I, NO₂, CHO, COOH, COO—(C₁-C₆)alkyl, CN and COCl.
192. The compound of embodiment 179 or 180, wherein n is 1 and R₁ a residue of formula (C)

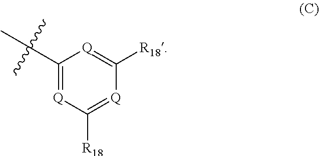

193. The compound of any of embodiments 179, 180, 192, wherein R₁₈ and R_{18′} are independently F, Cl, Br, I, or C₁-C₆ alkoxy and each Q is independently C or N, wherein at least one Q is N.
194. The compound of embodiment 179 or 180, wherein n is 1 and R₁ or a residue of formula (D)

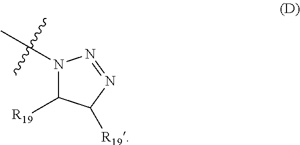

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the aromatic ring is preferably benzo,
wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl,
195. The compound of any of embodiment 179, 180, 194, wherein the substituent of the optionally substituted 5-, 6-, or 7-membered saturated or partially unsaturated or aromatic ring is at least a substituent, preferably one substituent, selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
196. The compound of any of embodiments 179, 180, 194 or 195, wherein the aromatic ring formed by R₁₉ and R_{19′} taken together is a benzo substituted with at least one, preferably with one substituent, wherein the substituent is selected from the group consisting of OH, C₁-C₆ alkoxy, aryl, heteroaryl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl), CN, NO₂, —NH₂, NR₂₇R₂₈, wherein R₂₇ and R₂₈ are independently selected from the group consisting of H, C₁-C₆ alkyl, C₁-C₆ alkoxy, aryl, heteroaryl, and wherein aryl at each occurrence is preferably phenyl.
197. The compound of any of embodiments 179 to 189, wherein Y is O and R₁ is the residue of formula (A).
198. The compound of any of embodiments 179 to 197 having formula (II-A)

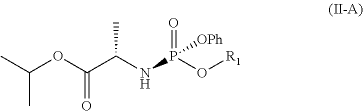

199. The compound of any of embodiments 179 to 197, having formula (II-B)

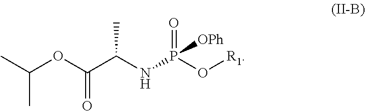

200. The compound of any of embodiments 179 to 199, wherein R₁ is a residue of formula (IIb)

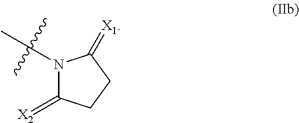

201. The compound of any of embodiments 179 to 199, wherein R₁ is a residue of formula (IIc)

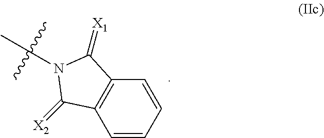

202. The compound of embodiment 200 or 201, wherein X₁ is O and X₂ is O.
203. Use of a Lewis acid, preferably of a Lewis acid comprising a twice positively charged ion or a three times positively charged ion, more preferably of a Lewis acid comprising a twice positively charged metal ion or a three times positively charged metal ion, for the preparation of a compound of formula (I),

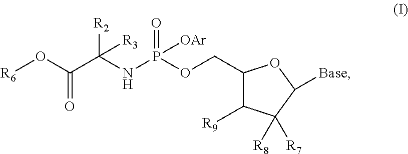

preferably for a compound of formula (I-A)

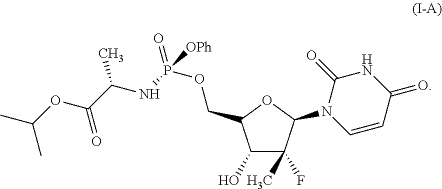

204. The use of embodiment 203, wherein the twice positively charged ion is a Zn ion, a Mg ion, a Cu ion, or an Fe ion, preferably a Zn ion, and wherein the three times positively charged ion is a Mn ion.
205. The use of embodiment 203 or 204 wherein the Lewis acid is one or more of one or more of ZnBr₂, ZnCl₂, ZnI₂, MgBr₂, MgBr₂. OEt₂, CuCl₂, Cu(acetylacetonate)₂, Fe(II) fumarate, Mn(acetylacetonate)₃, preferably one or more of ZnBr₂, ZnCl₂, ZnI₂, more preferably ZnBr₂.
206. Use of the compound of formula (I)

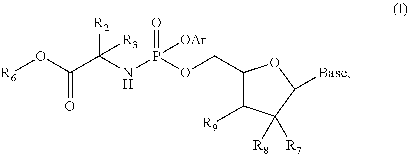

preferably of formula (I-A)

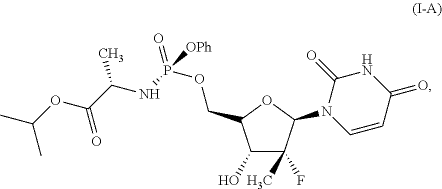

obtainable or obtained by a process according to any of embodiments 1 to 125, preferably 67 to 125, for the preparation of a pharmaceutical composition.
207. A pharmaceutical composition, comprising the compound of formula (I)

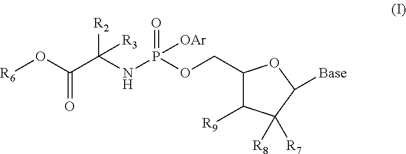

preferably the compound of the formula (I-A),

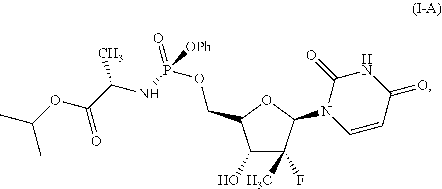

obtainable or obtained by a process according to any of embodiments 1 to 125, preferably 67 to 125, and at least one pharmaceutically acceptable excipient.
208. The pharmaceutical composition of embodiment 207 for use in a method for treating hepatitis C in a human.

The present invention is further illustrated by the following examples and references examples.

### EXAMPLES

### Reference Example 1: General Analytical Methods

Reactions were monitored by HPLC on a C-18 reverse phase column with a gradient of acetonitrile in 10 mM ammonium sulfamate aqueous buffer at pH 5.6. The diastereomeric ratio of N-hydroxysuccinimide phosphoramidate (II-a) was measured after methanolytic derivatization by GC using an HP-5 column and a temperature gradient. NMR spectra were recorded in CDCl₃ on a 300 MHz spectrometer. ¹H and ¹³C chemical shifts are given in ppm relative to TMS (0 ppm) with the solvent resonance as the internal standard (CDCl₃, ¹H: 7.26 ppm, ¹³C: 77.16 ppm).

### Example 1: Synthesis of Intermediate Compounds of Formula (II)

### 1.1 General Procedure for the Synthesis of Isopropyl(Phenoxy)Phoshoryl)Aminopropanoates Intermediates of Formula (II)

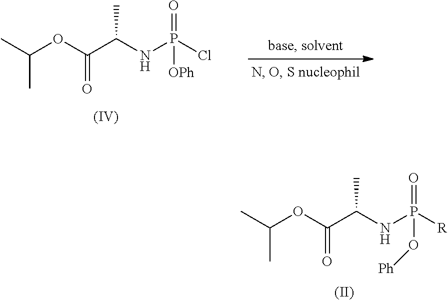

To a solution of (2S)-isopropyl 2-((chloro(phenoxy)phosphoryl)amino)propanoate (IV) (prepared according to the reference J. Org. Chem 2011, 76, 8311) in MTBE or methylene chloride, 0.85 eq of a N, S or O nucleophile were added (e.g. N-hydroxysuccinimmide, oxazoli-dinone derivatives, hydroxyl pyridines or thiophenols). In addition, 1.1 eq of an amine base (e.g. triethylamine) were added and the reaction mixture was stirred until complete conversion was observed. The ammonium salt was removed (if precipitation occurred) by filtration. Aqueous work-up was carried out under slightly acidic conditions (e.g. aqueous ammonium chloride) promoting removal of left over salts of the amine base. Removal of the solvent led to the corresponding phosphoramidate as a diastereomeric mixture. Purification was possible via crystallization depending on the nature of the leaving group.

1.2 Synthesis of the Compound of Formula (II-a) with N-Hydroxysuccinimide as LG and Crystallization Thereof to Obtain Compound of Formula (II-a′).

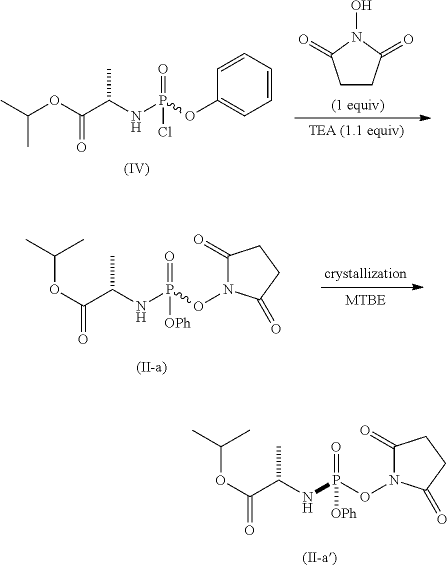

In a dry two-neck round bottom flask equipped with a dropping funnel was dissolved crude phosphoryl chloride (IV) prepared according J. Org. Chem 2011, 76, 8311 (20 g, 43.8 mmol, 1 equiv, 67% w/w purity by NMR) in dichloromethane (140 mL) and the solution was cooled to ca. 5° C. with an ice bath. N-hydroxysuccinimide (7.53 g, 65.4 mmol, 1 equiv) was added (only partial dissolution). To this suspension, triethylamine (10 mL, 71.9 mmol, 1.1 equiv) in dichloromethane (20 mL) was added dropwise with stirring, and the dropping funnel was rinsed with a further 5 mL of dichloromethane, whereby all of N-hydroxysuccinimide dissolved and a precipitation of triethylamine hydrochloride was observed. The ice bath was removed, the reaction mixture was allowed to warm up to room temperature and extracted with 90 mL of distilled water. The organic phase was washed with a further 40 mL of distilled water and the volatiles were removed under reduced pressure. The crude solid was dissolved in 160 mL MTBE (methyl tert-butyl ether), charged with 5 mL triethylamine and left to stir overnight, upon which a solid agglomerate was formed. The mixture was diluted with 75 mL of MTBE and warmed up to 50° C. until all of the solid dissolved. Upon cooling, crystals formed which were filtered and dried to give 10.53 g of (II-a′) (dr 68.5:31.5). This solid was suspended in 155 mL MTBE, warmed to 45° C. until complete dissolution and allowed to cool, whereby white needle crystals formed. The crystals were filtered and dried to give 3.09 g diastereopure (II-a′). The mother liquor was evaporated under reduced pressure, dissolved in 80 mL MTBE with heating and concentrated to 55 mL under reduced pressure. After 45 min of stirring, white crystals formed, which were filtered and dried to give 1.11 g diastereopure (II-a′), to give a total yield of 4.19 g of (II-a) (10.9 mmol, 25%). The diastereomeric purity of the product was determined by GC analysis after methanolytic derivatization in the following way: in a vial, 21.4 mg of the solid were dissolved in 1 mL of a 1.25M HCl solution in MeOH, the vial was capped, shaken and directly used for a GC injection.

1.3 One Pot Synthesis of Compound of Formula (II-a) and Crystallization Thereof to Obtain the Compound of Formula (II-a′) Starting from Phenyl Dichlorophosphate

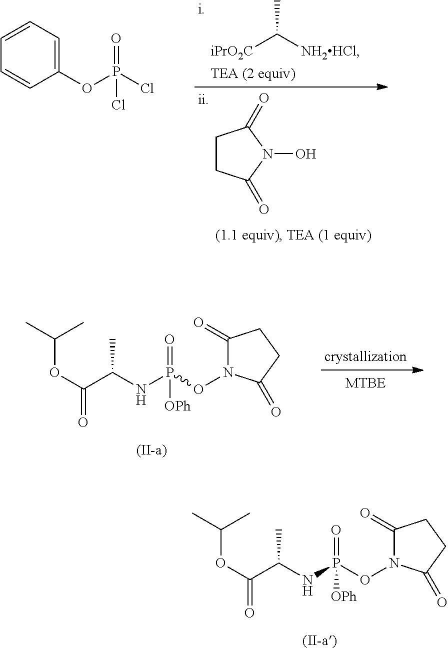

In a dry two-neck round bottom flask equipped with a mechanical stirrer and a dropping funnel was dissolved L-alanine isopropyl ester (20.0 g, 119 mmol, 1 equiv) in dichloromethane (125 mL) and the solution was cooled to −78° C. with a dry ice/acetone bath. To this solution, triethylamine (33 mL, 239 mmol, 2 equiv) was added via a dropping funnel with stirring, upon which a white precipitate was formed. Phenyl dichlorophosphate (sourced by Aldrich) (17.8 mL, 119 mmol, 1 equiv) in dichloromethane (125 mL) was then added dropwise over 1 h, and the reaction mixture was stirred for 30 min at −75° C. and for 2 h at 0° C. In a separate flask, N-hydroxysuccinimide (13.68 g, 119 mmol, 1 equiv) was suspended in dichloromethane (75 mL) and charged with triethylamine (16.5 mL, 119 mmol, 1 equiv) upon which a solution was obtained. This solution was added to the main reaction vessel dropwise over 40 min.

The reaction mixture was allowed to warm up to room temperature and stirred overnight. The crude reaction mixture was filtered washing with dichloromethane and extracted with a 1:1 mixture of sat. aq. NH₄Cl and water (1×200 mL and 1×100 mL), followed by a 1:1 mixture of saturated aqueous NaCl and water (1×100 mL). The organic phase was separated and the volatiles were removed under reduced pressure. The crude oil was dissolved in 160 mL MTBE and seeded with pure (II-a′) and stirred, upon which a solid began to form slowly. The mixture was diluted with 100 mL of MTBE, warmed up until all of the solid dissolved and seeded with pure (II-a′) again, upon which needle-like crystalline solid began to form slowly. The mixture was diluted with 100 mL MTBE and left to stand overnight, then stirred at 0° C. in an ice bath. The solid was filtered and dried to give 3.25 g diastereopure (II-a) (8.4 mmol, 7%).

¹H NMR (300 MHz, CDCl₃): 7.41-7.29 (m, 4H), 7.25-7.17 (m, 1H), 5.03 (sept, J=6.2 Hz, 1H), 4.29-4.13 (m, 1H), 4.09 (dd, J=11.2 Hz, 9.8 Hz, 1H), 2.78 (s, 4H), 1.44 (d, J=7.0 Hz, 3H), 1.26 (apparent t, J=6.65 Hz, 6H).

¹³C NMR (75 MHz, CDCl₃): 173.0 (d, J=7.6 Hz), 169.4, 150.4 (d, J=7.5 Hz), 129.9, 125.7, 120.2 (d, J=5.1 Hz), 69.5, 50.6 (d, J=2.3 Hz), 25.6, 21.8 (J=2.8 Hz), 20.8 (d, J=5.6 Hz).

1.4 Synthesis of the Compound of Formula (II-a′) with N-Hydroxysuccinimide as LG: Aqueous Workup

20 g alaninisopropyl ester hydrochloride (119.3 mmol) was charged with 200 ml of dry THF and cooled to 0° C. To the mixture 21.4 ml phenylphosphorodichlorate (content: 95%, 131.2 mmol, 1.1 eq) was added and the mixture was stirred for 15 min at 0° C. To the mixture 34.8 ml triethyl amine (250-5 mmol, 2.1 eq) was added within 77 min while the reaction mixture was kept at temperature below 6° C. After complete addition the mixture was stirred for an additional hour at 0° C. until complete conversion was observed. To the mixture 17.8 g N-hydroxysuccinimide (155.09 mmol, 1.3 eq) as solid was added. Afterwards 24.8 ml triethyl amine (179.0 mmol, 1.5 eq) was slowly added to keep the mixture temperature below 5° C. The mixture was stirred for 2.5 h after complete addition until complete conversion was observed. To the mixture 1.2 L MTBE was added and afterwards the mixture was hydrolyzed with 1.2 L water. The organic phase was separated and washed with 200 ml brine. The organic phase was stripped with 4 times with 900 ml MTBE until no THF was detectable via GC in the organic phase. The total volume of the organic phase was adjusted to 920 ml by addition of MTBE. The mixture was heated to 42° C. and to the mixture 1.0 g N-hydroxysuccinimide (8.6 mmol, 7.5 mol %) and 5.0 ml trimethylamine (35.8 mmol, 0.3 eq) was added. The mixture was cooled to 27° C., when precipitation was observed. At that point, seeds of diastereomerically pure N-hydroxysuccinimide phosphoramidate (II-a′) were added. The mixture was stirred cooled to 0° C. over a period of 4 hours and afterwards stirred at 0° C. overnight. The precipitation was isolated via filtration and dried at <100 mbar at 20-25° C. leading to 24.5 g of N-hydroxysuccinimide phosphoramidate (II-a′) with a diastereomeric ratio of 88/12. The mixture was further purified by crystallization from MTBE leading to N-hydroxysuccinimide phosphoramidate (II-a′) with a diastereomeric ratio of >97/3.

1.5 Synthesis of the Compound of Formula (II-a′) with N-Hydroxysuccinimide as LG Water Free Workup

20 g alaninisopropyl ester hydrochloride (119.3 mmol) was charged with 200 ml of dry THF and cooled to 0° C. To the mixture 17.1 ml phenylphosphorodichlorate (content: 95%, 107.4 mmol, 0.9 eq) was added and the mixture was stirred for 15 min at 0° C. To the mixture 34.8 ml triethyl amine (250-5 mmol, 2.1 eq) was added within 65 min while the reaction mixture was kept at temperature below 7° C. After complete addition the mixture was stirred for two additional hour at 0° C. until complete conversion was observed. To the mixture 17.8 g N-hydroxysuccinimide (155.09 mmol, 1.3 eq) as solid was added. Afterwards 24.8 ml triethyl amine (179.0 mmol, 1.5 eq) was slowly added to keep the mixture temperature below 7° C. The mixture was stirred for 2 h after complete addition until complete conversion was observed. To the mixture 200 ml MTBE was added an the mixture was stirred at 0° C. for 30 min. Afterwards the precipitation was removed (57.5 g, containing mainly triethylamine hydrochloride, N-hydroxysuccinimide salts with triethylamine and small amounts of the desired phosphoramidate). The volume of the filtrate was reduced to 59 g (still liquid with suitable viscosity for stirring) and 900 ml MTBE was added. After the addition of MTBE a precipitation occurred. The mixture 30 g silica gel was added and the mixture was stirred for 10 min. The precipitation was removed via filtration and the filter cake was washed with 100 ml MTBE to achieve a clear solution. The volume of the filtrate was reduced to 600 ml and heated to 30° C. At this temperature 5.0 ml triethyl amine was added and the mixture was cooled to 20° C. At this temperature seeds of the diastereomeric pure N-hydroxysuccinimide phosphoramidate (II-a′) were added. The mixture was cooled to 10° C. and stirred at that temperature for 3.5 h. Afterwards the mixture was cooled to 0° C. and stirred for 8 h at this temperature. The precipitation was isolated via filtration and the filter cake was washed with 30 ml of MTBE/HPT mixture (1/1). After drying at <100 mbar/40° C., 16.4 g of N-hydroxysuccinimide phosphoramidate (II-a′) with a diastereomeric ratio of 89/11 was isolated.

### 1.6 Crystallization of Diastereomeric Pure N-Hydroxysuccinimide Phosphoramidate (II-a′)

16.4 g N-hydroxysuccinimide phosphoramidate (II-a′) (dr=9/1) are added to 246 ml MTBE and the mixture was heated to 40° C. The occurring suspension was cooled to 20° C. and stirred at that temperature for 2 h. Afterwards the mixture was cooled to 0° C. and stirred at this temperature for an additional hour. The precipitation was isolated via filtration. After drying (<100 mbar, 20-40° C.), 15.8 g N-hydroxysuccinimide phosphoramidate (II-a′) in diastereomeric pure form was isolated.

1.7 Synthesis of Compound of Formula (II-b) and Purification to Obtain the Diasteromerically Pure Compound (II-b′): (2S)-isopropyl 2-(((2-oxobenzo[d]oxazol-3(2H)-yl)(phenoxy)phosphoryl)amino)propanoate (Oxazolidone as Nuclephile)

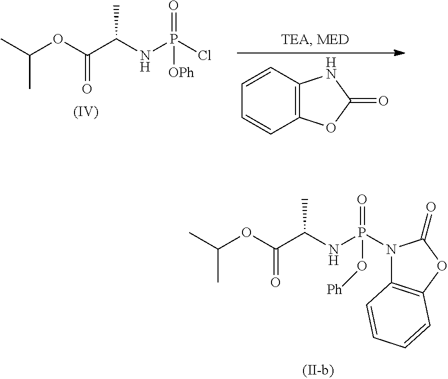

20 g (65.4 mmol, 1 eq) of (2S)-isopropyl 2-((chloro(phenoxy)phosphoryl)amino)propanoate was dissolved in 500 ml methylene chloride. To the solution 7.5 g (55.5 mmol, 0.85 eq) 2-benzoxazolinone and 10 ml (72.1 mmol, 1.1 eq) triethyl amine were added. Complete conversion was observed after 3 h. The solvent was removed to dryness and the residue was suspended in 400 ml MTBE. The occurring solid was removed and the filtrate was washed with aqueous ammonium chloride and brine. The organic solvent was again removed to dryness and 22.9 g of an oil were isolated. The residue was treated with 40 mL ethyl acetate. After removal of 30 mL ethyl acetate via distillation a suspension was formed. The suspension was treated with 30 mL heptane and after 1 h stirring the solid was isolated via filtration. After drying 6.9 g were isolated as a diastereoisomerically pure product (II-b′) having configuration S at the “P” atom. From the mother liquor 13.9 g of the product were isolated after removal of the solvent as a mixture of diastereoisomers. Characterization of the product:

¹H NMR (300 MHz, CDCl₃): 7.76-7.32 (m, 1H), 7.29-7.26 (m, 5H), 7.18-7.11 (m, 5H), 5.02 (sept, J=7.0 Hz, 1H), 4.48-4.32 (m, 2H), 1.38 (d, J=7.6 Hz, 3H), 1.25-1.21 (m, 6H).

1.8 Synthesis of Phosphoramidates of Formula (II-c) from Phosphoric Acid Derivatives

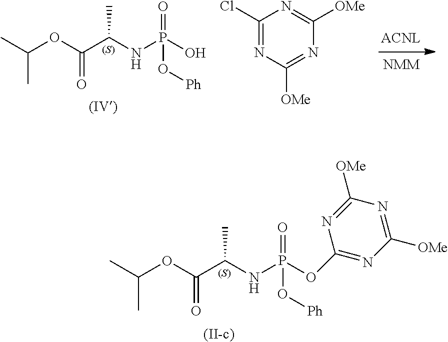

Step 1: Synthesis of (2S)-isopropyl 2-((hydroxy(phenoxy)phosphoryl)amino)propanoate of formula (IV′)

To a suspension of 2.0 g of alanine isopropylester hydrochloride in 30 ml THF, 2.29 g EDC.HCl (1 eq), 2.08 g phenylphosphoric acid (1.0 eq) and 2.6 mL N-methylmorpholine (2 eq) were added. The mixture was stirred for 3.5 h until complete conversion was observed. The reaction was hydrolyzed with 40 ml aqueous ammonium chloride and extracted with 50 mL methylene chloride. The aqueous phase was re-extracted with 30 mL methylene chloride and the solvent was removed from the combined organic phases. The product was crystallized from acetonitrile leading to 920 mg of the desired product (2S)-isopropyl 2-((hydroxy(phenoxy)phosphoryl)amino)propanoate. Characterization of the product:

¹H NMR (300 MHz, CDCl₃): 8.2 (b, 1H) 7.20-6.87 (m, 5H), 4.99-4.76 (m, 1H), 3.91-3.48 (m, 1H), 3.68 (b, 1H) 1.37-1.06 (m, 9H).

### Step 2: Synthesis of Phosphoramidates of Formula (II-c)

3.2 g (2S)-isopropyl 2-((hydroxy(phenoxy)phosphoryl)amino)propanoate (11.14 mmol) (prepared according to step 1) were dissolved in 60 mL acetonitrile. To the solution 2.42 g 2-chloro-4,6-dimethoxy-1,3,5-triazin (1.2 eq) and 2.4 mL N-methylmorpholine (2 eq) were added. After compete conversion, the solvent was removed to dryness and the residual was dissolved in 100 mL methylene chloride. The organic phase was washed with 100 mL water and brine and the organic solvent was removed to dryness. 4.7 g of an orange oil were isolated containing 61.5 area-% of the desired product (2S)-isopropyl 2-((((4,6-dimethoxy-1,3,5-triazin-2-yl)oxy)(phenoxy)phosphoryl)amino)propanoate. Characterization of the product:

¹H NMR (300 MHz, CDCl₃): 7.25-7.09 (m, 5H), 5.97 (m, 1H), 4.99 (m, 1H), 4.58 (m, 1H), 3.87 (s, 9H) 1.42 (d, J=7.9 Hz, 3H), 1.20-1.66 (m, 6H).

### Example 2: Synthesis of Compound of Formula (I-S) Sofosbuvir with Lewis Acid (ZnBr₂) by Reaction of Compound of Formula (III-S)

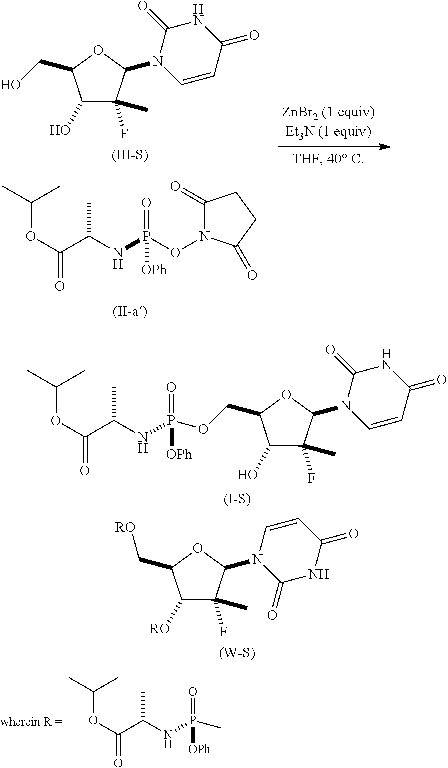

### 2.1 Synthesis of Compound (I-S): 0.195 Mmol Scale

The preparation of compound (III-S) was according to the glycosylation of the nucleobase disclosed in patent application WO2005/003147 and J. Med. Chem. 2005, 48, 5504. To a two-neck round bottom flask equipped with a reflux condenser and purged with nitrogen was added 2′-deoxy-2′-fluoro-2′C-methyluridine (III-S) (50.8 mg, 0.195 mmol, 1 equiv), anhydrous THF (3 mL) and 4 Angstrom molecular sieves (powder, 1 heaped spatula tip), and the suspension was stirred for 5 min. N-hydroxysuccinimide phosphoramidate (II-a′) (150 mg, 0.390 mmol, 2 equiv, stored in a desiccator and checked periodically for hydrolysis) was added, followed by ZnBr₂ (43.9 mg, 0.195 mmol, 1 equiv), and Et₃N (27.2 microL, 0.195 mmol, 1 equiv). The mixture was heated at 40° C. for 2 hours. HPLC analysis with individual response factor correction indicated 11% of unreacted nucleoside (III-S), 82% (I-S) (97:3 dr) and 7% of 3′,5′-bis-phosphoramidate impurity (W-S) which represents the wrong region-isomers formed during the nucleophilic substitution reaction (dr not determined). “(dr)” is the diastereoisomer ratio when referring to two diastereoisomers that differ for the phosphorpus “P” chirality.

2.2 Synthesis of Compound (I-S): 5.76 mmol Scale

To a two-neck round bottom flask equipped with a reflux condenser and purged with nitrogen was added 2′-deoxy-2′-fluoro-2′C-methyluridine (III-S) (1.49 g, 5.76 mmol, 1 equiv), anhydrous THF (90 mL), ZnBr₂ (1.31 g, 5.76 mmol, 1 equiv) and 4 Angstrom molecular sieves (9.0 g, powder), and the suspension was stirred for 10 min. N-hydroxysuccinimide phosphoramidate (II-a′) (4.44 g, 11.55 mmol, 2 equiv, stored in a dessicator and checked periodically for hydrolysis) was added in one portion, followed a dropwise addition of Et₃N (0.8 mL, 5.76 mmol, 1 equiv). The mixture was heated at 40° C. for 23 hours, at which point HPLC analysis with individual response factor correction showed 8% of unreacted nucleoside (III-S), 88% of (I-S) (92:8 dr) and 4% of 3′,5′-bis-phosphoramidate impurity (dr not determined).

The crude reaction mixture was filtered, washed with THF (about 3 mL), and the solvent was removed under reduced pressure. The resulting amorphous solid was redissolved in 75 mL isopropyl acetate and extracted with a 1:1 mixture of saturated aqueous NaCl and water (1×40 mL), followed by of sat. aq. NaHCO₃ (2×40 mL). The organic phase was separated and dried over Na₂SO₄, filtered, and the solvent was removed under reduced pressure to give a white amorphous solid (6.98 g). Of this crude solid, 996 mg were dissolved in toluene (2 mL) and charged with MTBE (2 mL). The mixture was seeded with pure (I-S) which resulted in immediate precipitation of a white crystalline solid. The mixture was stirred overnight under nitrogen, filtered washing with a 1:1 mixture of MTBE/dichloromethane (2×1 mL) and dried under vacuum at 50° C. overnight to give (I-S) as a white crystalline solid (350 mg, 0.66 mmol, 80% yield for the recrystallized portion).

HPLC analysis showed a total purity of 96.1%, with impurities composed of the other diastereoisomer of compound of formula (I) with R configuration at the “P” atom (I-R). (1.7%), N-hydroxysuccinimide (0.4%), hydrolysis product of the N-hydroxysuccinimide phosphoramidate (II-a′) (0.2%), N-hydroxysuccinimide phosphoramidate (II-a′) (0.5%) and an unknown impurity (1.1%). Characterization of the product:

¹H NMR (300 MHz, CDCl₃): 9.41 (br s, 1H), 7.47 (d, J=8.2 Hz, 1H), 7.38-7.28 (m, 2H), 7.25-7.13 (m, 3H), 6.17 (br d, J=18.7 Hz, 1H), 5.69 (d, J=8.1 Hz, 1H), 5.00 (sept, J=6.2 Hz, 1H), 4.60-4.36 (m, 2H), 4.24 (dd, J=11.9 Hz, 10.0 Hz, 1H), 4.15-4.03 (m, 2H), 4.03-3.83 (m, 2H), 1.38 (d, J=18.9 Hz, 3H), 1.33 (d, J=3.3 Hz, 3H), 1.22 (d, J=6.2 Hz, 6H).

### 2.3 Synthesis of Sofosbuvir (Compound (I-S))

7.58 g N-hydroxysuccinimide phosphoramidate (II-a′) (85% content, dr 08.4/1.6, 16.8 mmol, 1.12 eq) was dissolved in 49 ml dry THF. To the mixture 2′-deoxy-2′-fluoro-2′C-methyluridine (III-S) (14.95 mmol, 1 eq) and 3.37 g zinc bromide (14.95 mmol, 1 eq) was added and stirred for 10 min. Afterwards 4.14 ml triethyl amine (29.9 mmol, 2 eq) was added. The mixture was stirred overnight at room temperature leading to a conversion of 98% and dr for sofosbuvir of 97/3. The precipitation of the reaction mixture was removed via filtration and the filter cake was washed with 8.5 ml THF. The acidic work up of the filtrate was done via addition of 25 ml 1M aqueous HCl leading to a clear solution. The THF was removed from the mixture via evaporation (80 mbar, 45° C.). Afterwards 50 ml methylene chloride 10 ml brine was added to the mixture. The organic phase was separated and the volume was reduced to 33.7 g via evaporation at 35° C. The organic solution was cooled to 30° C. and seeds of sofosbuvir were added. Precipitation started at 30° C. The mixture was cooled to −10° C. within 5.5 h and stirred at −10° C. overnight. The precipitation was isolated via filtration and the filter cake was washed with −20° C. cooled methylene chloride. After drying (<100 mbar, 20° C.), 5.49 sofosbuvir (81% yield, dr=99.7/0.3) was isolated.

### Example 3: General Procedure for Lewis Acids (0.192 mmol Scale)

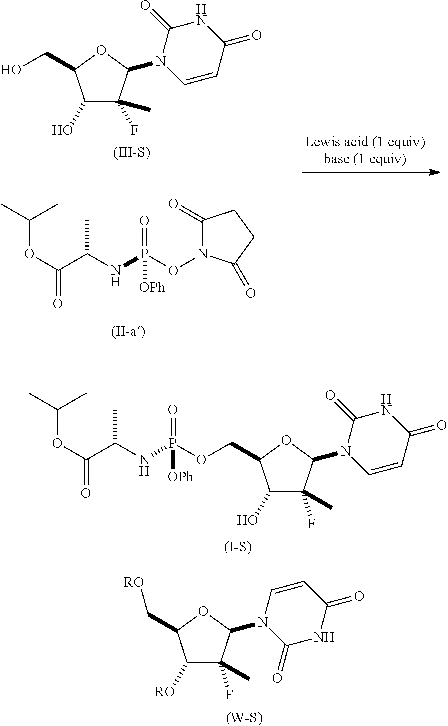

To a two-neck round bottom flask equipped with a reflux condenser and purged with nitrogen was added 2′-deoxy-2′-fluoro-2′C-methyluridine (III-S) (50 mg, 0.192 mmol, 1 equiv), 3 mL anhydrous THF and 4 Angstrom molecular sieves (powder, 1 heaped spatula tip), and the suspension was stirred for 5 min. N-hydroxysuccinimide phosphoramidate (II-a′) (148 mg, 0.384 mmol, 2 equiv unless specified otherwise) was added, followed by the Lewis acid (1 equiv), and Et₃N (26.8 microL, 0.192 mmol, 1 equiv, unless otherwise specified).

The mixture was heated at 40° C. for 1 to 22 hours and analyzed by HPLC, taking into account the response factor of each reactant and product.

The above experiment was repeated by varying the reaction conditions (Lewis acid, base, temperature, reaction time) as reported in entries 1 to 15 of Table 1 below. Table 1 reports also the percentage based on the total mole conversion of compounds (I-S) and (W-S) and the diastereoisomer ratio of compound (I-S) relative to its diastereoisomer (I-R) (different chirality of the “P” atom).

TABLE 1

| | | | | | | Total | | | |
|---|---|---|---|---|---|---|---|---|---|
| | | | | | | mole | | | |
| | Lewis acid, | Base, | (II-a′) | Temp/ | Time/ | convers./ | % | % | dr of |
| Entry | 1 eq | 1 eq | 1 eq | ° C. | h | % | (I-S) | (W-S) | (I-S) |
| 1 | Mn(acac)₃ | Et₃N | 1 | r.t.-40 | 17 | 8 | 8 | 0 | 62:38 |
| 2 | CuCl₂ | Et₃N | 1 | 40 | 20.5 | 6 | 6 | 0 | 78:22 |
| 3 | ZnI₂ | Et₃N | 1 | 40 | 20.5 | 12 | 12 | 0 | 80:20 |
| 4 | Fe (II) fumarate | Et₃N | 1 | 40-66 | 2.5 | 17 | 17 | 0 | 57:43 |
| 5 | Cu(acac)₂ | Et₃N | 1 | 40 | 1 | 12 | 12 | 0 | 54:46 |
| 6 | MgBr₂•OEt₂ | Et₃N | 1 | 40 | 22.5 | 9 | 7 | 2 | 97:3 |
| 7 | Mn(acac)₃ | none | 1 | 40 | 10 | 64 | 54 | 10 | 79:21 |
| 8 | Mn(acac)₃ | Et₃N | 1 | 40 | 5.5 | 58 | 52 | 5 | 86:14 |
| 9 | ZnCl₂ | Et₃N | 1 | 40 | 17 | 90 | 81 | 9 | 92:8 |
| 10 | ZnCl₂ | Et₃N | 2 | 40 | 21 | 100 | 86 | 14 | 95:5 |
| 11 | ZnBr₂ | Et₃N | 1 | 40 | 20 | 94 | 86 | 8 | 93:7 |
| 12 | ZnBr₂ | Et₃N | 2 | r.t. | 18 | 92 | 86 | 6 | 98:2 |
| 13¹⁾ | ZnBr₂ | Et₃N | 1 | 40 | 22 | 71 | 67 | 4 | 90:10 |
| 14 | ZnBr₂ | DBU | 1 | 40 | 22 | 91 | 84 | 7 | 93:7 |
| 15 | ZnBr₂ | pyridine | 1 | 40 | 18 | 41 | 40 | 1 | 72:28 |
| 16²⁾ | ZnBr₂ | Et₃N | 1.12 | r.t | over | 98% | | | 99.7:0.3 |
| | | (2 eq) | | | night | | | | |

¹⁾Reaction performed in DMF
²⁾Reaction according to example 2.3

As can be seen from the results reported in Table 1 with all tested Lewis acids according to the present invention, no prevalence of the wrong regioisomer (W-S) has been observed.

In some cases (entries 1 to 5), no formation of the wrong regioisomer has been detected.

Additionally, in all cases, a high percentage of the correct regioisomer (I-S) has been observed.

### Comparative Example 1: Synthesis of Compound (I-S) with Grignard

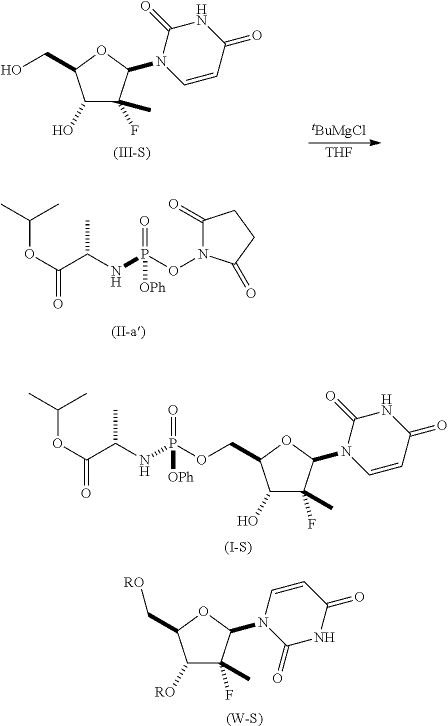

To a dry two-neck round bottom flask equipped with a reflux condenser and purged with nitrogen was added 2′-deoxy-2′-fluoro-2′C-methyluridine (III-S) (52.3 mg, 0.20 mmol, 1 equiv), 2 mL anhydrous THF and 4 Angstrom molecular sieves (powder, 1 heaped spatula tip), and the suspension was stirred for 5 min. Tert-butyl magnesium chloride (0.4 mL, 0.40 equiv, 2 equiv, 1 M in THF) was then added dropwise, upon which a thick white precipitate formed. The suspension was stirred for 10 min and N-hydroxysuccinimide phosphoramidate (II-a′) (154.5 mg, 0.40 mmol, 2 equiv, stored in a desiccator and periodically checked for hydrolysis) in anhydrous THF (1 mL) was added in one portion. The mixture was heated at 40° C. for 6 hours. HPLC analysis with individual response factor correction indicated 8% of unreacted nucleoside (III-S), 21% (I-S) (93:7 dr) and 71% of 3′,5′-bis-phosphoramidate impurity (W-S) (dr not determined). The results are given in Table 2 (entry 1). The above experiment was repeated with different reaction conditions (Grignard reagent, reaction time) as reported in entries 2 and 3 of Table 2. Table 2 reports also the percentage based on the total mole conversion of compounds (I-S) and (W-S) and the diastereoisomer ratio of compound (I-S) relative to its diastereoisomer (different chirality of the “P” atom)

TABLE 2

| | tert- | | | | | | |
|---|---|---|---|---|---|---|---|
| | BuMgCl | (II-a′) | | Total mole | % | % | dr of |
| Entry | 1 eq | 1 eq | Time/h | convers./% | (I-S) | (W-S) | (I-S) |
| 1 | 2 | 2 | 6 | 92 | 21 | 71 | 93:7 |
| 2 | 2 | 1 | 1 | 27 | 19 | 8 | 98:2 |
| 3 | 1 | 1 | 19.5 | 39 | 24 | 16 | 97:3 |

In comparison with the process according to the invention, it can be seen in Table 2-entry 1 that the wrong regioisomer (W-S) is formed in higher percentage with respect to valuable compound (I-S). In general with Grignard reagents either a high conversion is observed that however leads to the prevalence of the wrong regioisomer (W-S) or a low conversion is observed. With the Lewis acid reaction according to the present invention, no prevalence of the wrong regioisomer (W-S) has been observed.

### Comparative Examples 2: Synthesis of Compound 3 with Diethyl-Zinc

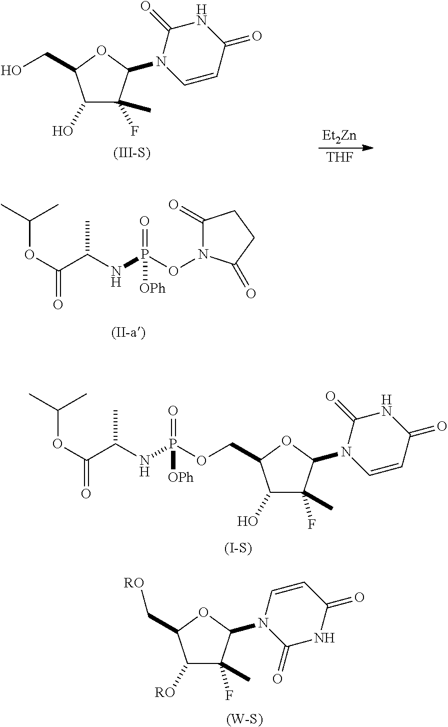

To a dry two-neck round bottom flask equipped with a reflux condenser and purged with nitrogen was added 2′-deoxy-2′-fluoro-2′C-methyluridine (III-S) (51.4 mg, 0.198 mmol, 1 equiv), 2 mL anhydrous THF, and Et₂Zn (198 microL, 0.198 mmol, 1 equiv, 1M in hexane) and the suspension was stirred for 10 min. N-hydroxysuccinimide phosphoramidate (II-a′) (76.1 mg, 0.198 mmol, 1 equiv) in anhydrous THF (1 mL) was then added in one portion. The mixture was heated at 40° C. for 19.5 hours. HPLC analysis with individual response factor correction indicated 39% of unreacted nucleoside (III-S), 48% (I-S) (92:8 dr) and 13% of 3′,5′-bis-phosphoramidate impurity (W-S) (dr not determined). The results are given in Table 3 (entry 1). The above experiment was repeated with different reaction times as reported in entries 2 to 4 of Table 3. Table 3 reports also the percentage based on the total conversion of compounds (I-S) and (W-S) and the diastereoisomer ratio of compound (I-S) relative to its diastereoisomer (different chirality of the “P” atom)

TABLE 3

| | Et₂Zn | | Total mole | % | % | dr of |
|---|---|---|---|---|---|---|
| Entry | 1 eq | Time/h | conversion/% | (I-S) | (W-S) | (I-S) |
| 1 | 0.5 | 5 | 47 | 36 | 11 | 88:12 |
| 2 | 1.25 | 2 | 63 | 38 | 25 | 77:23 |
| 3 | 3 | 4 | 15 | 10 | 5 | 41:59 |

In comparison with the process according to the invention, it can be appreciated that with the reaction according to the invention compound (I-S) is formed in higher percentage with respect to the wrong regioisomer (W-S).

### Comparative Example 3: Synthesis of Compound (I-S) with Tert-BuZnBr and Further Bases

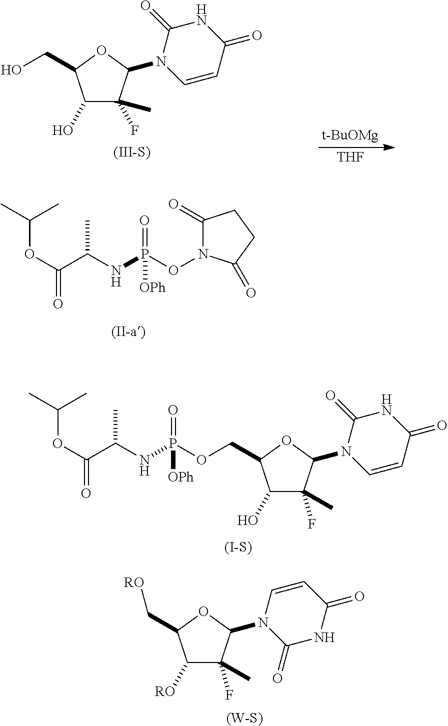

To a dry two-neck round bottom flask equipped with a reflux condenser and purged with nitrogen was added 2′-deoxy-2′-fluoro-2′C-methyluridine (III-S) (51.6 mg, 0.198 mmol, 1 equiv), 2 mL anhydrous THF, and 4 Angstrom molecular sieves (powder, 1 heaped spatula tip), and the suspension was stirred for 5 min. tert-BuZnBr (790 microL, 0.397 mmol, 2 equiv, 0.5 M in THF) was added dropwise, whereby the reaction mixture remained homogeneous. N-hydroxysuccinimide phosphoramidate (II-a′) (76.1 mg, 0.198 mmol, 1 equiv) in anhydrous THF (1 mL) was then added in one portion. The mixture was heated at 40° C. for 2 hours (a precipitate was formed after 1 hour reaction time) and analyzed by HPLC, calculating for the response factor of each reactant and product. HPLC indicated 22% of unreacted nucleoside (III-S), 66% (I-S) (82:18 dr) and 12% of 3′,5′-bis-phosphoramidate impurity (W-S) (dr not determined). The results are given in Table 4 (entry 1). The above experiment was repeated with different reaction conditions (base and reaction time) as reported in entries 2 to 5 of Table 4. Table 4 reports also the percentage based on the total conversion of compounds (I-S) and (W-S) and the diastereoisomer ratio of compound (I-S) relative to its diastereoisomer (different chirality of the “P” atom)

TABLE 4

| | | | | Total mole | % | % | dr of |
|---|---|---|---|---|---|---|---|
| Entry | Base | eq | Time/h | conversion/% | (I-S) | (W-S) | (I-S) |
| 1 | t-BuOMg | 2 | 2.5 | 55 | 34 | 21 | 90:10 |
| 2 | Me₃Al | 1 | 3 | 24 | 12 | 13 | 95:5 |
| 3 | Bu₂Mg | 1 | 3 | 50 | 24 | 24 | 95:5 |

| | | | |
|---|---|---|---|
| 4 | n-BuLi | 1 | no reaction |
| 5 | MgH₂ | 1 | no reaction |

In Table 4-entries 4 and 5 no reaction has been observed. Additionally in Table 4-entry 2 the wrong regioisomer (W-S) in former in higher percentage with respect to valuable compound (I-S) while in table Table 4-entry 4 the same % of (I-S) and (W-S) is obtained.

In comparison with the process according to the invention it can be appreciated that in the reaction according to the invention compound (I-S) is formed in higher percentage with respect to the wrong regioisomer (W-S).

### Comparative Example 4: Synthesis of Compound (I-S) in HCl

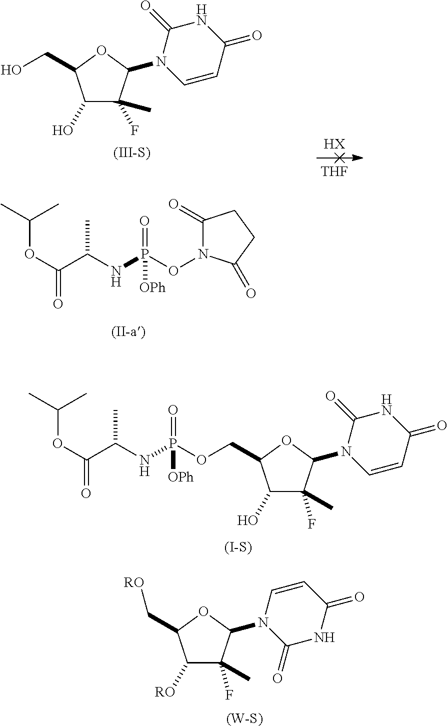

To a dry two-neck round bottom flask equipped with a reflux condenser and purged with nitrogen was added 2′-deoxy-2′-fluoro-2′C-methyluridine (III-S) (50.12 mg, 0.193 mmol, 1 equiv), 3 mL anhydrous THF, and 4 Angstrom molecular sieves (0.3 g, powder), and the suspension was stirred for 10 min. 4M HCl in dioxane (48 microL, 0.193 mmol, 1 equiv) was added, followed by N-hydroxysuccinimide phosphoramidate (II-a′) (74.0 mg, 0.193 mmol, 1 equiv). The mixture was heated at 40° C. for 1 hour, and analyzed by HPLC which indicated no conversion. Triethylamine (24.7 microL, 0.193 mmol, 1 equiv) was added and the reaction was further stirred overnight at 40° C. HPLC analysis with individual response factor correction indicated 94% of unreacted nucleoside (III-S) and 6% (I-S)(47:53 dr).

Using the same procedure but employing TFA as the Bronsted acid, similar results were obtained (13% conversion to (I-S), 57:43 dr after 19.5 h).

As can be seen, using an acid different from the present Lewis acid leads to a very low conversion with a dr ratio in favor of the wrong diastereoisomer (R configuration at the “P”).

### Example 4: Synthesis of Sofosbuvir Starting from Compound (II-b)

### 4.1 Lewis Acid Mediated and 2-Benzo[d]Oxazole as LG

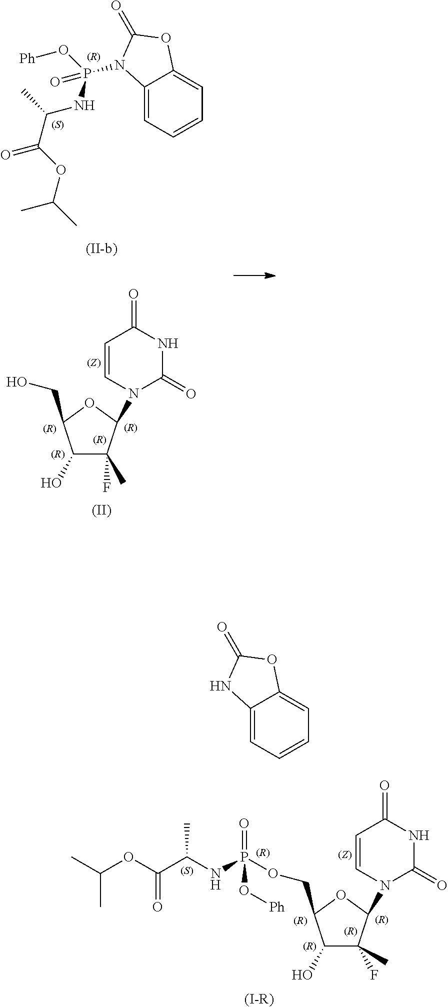

To a solution of 155.3 mg (0.48 mmol) of (2S)-isopropyl 2-(((2-oxobenzo[d]oxazol-3(2H)-yl)(phenoxy)phosphoryl)amino)propanoate in 2 ml THF 14 mg Mn(III)acetylacetonate (10 mol-%) were added. Afterwards 100 mg (0.38 mmol) deoxy-fluoro-methyl uridine were added and rinsed with 1 mL THF. To the reaction mixture 50 microL triethylamine were added and the mixture was heated to reflux for 44 h until no starting material was observed anymore. After cooling to room temperature the mixture was hydrolyzed with 7 mL aqueous ammonium chloride and extracted with 5 mL ethyl acetate. The organic phase contained a ½ mixture of two phosphor isomers of compound of formula (I) i.e. compound of formula (I-S) and diasteroisomer with the “P” having an R configuration (I-R).

### Comparative Example 5: Synthesis of Sofosbuvir Starting from Compound (II-b′) and Grignard Mediated

To a solution of 150 mg deoxy-fluoro-methyl uridine (0.58 mmol) in 3 mL THF, 1.2 mL tert-butyl magnesium chloride solution in THF (1 M, 1.2 eq) were added. The occurring suspension was stirred for 30 min. Afterwards 261 mg (1.2 mmol, 2.1 eq)) of (2S)-isopropyl 2-(((2-oxobenzo[d]oxazol-3(2H)-yl)(phenoxy)phosphoryl)amino)propanoate dissolved in 2 mL THF were added. After complete conversion the reaction was hydrolyzed by the addition 10 mL aqueous ammonium chloride and extracted with 20 mL ethyl acetate. The organic phase was washed with 5% sodium carbonate solution and brine. After removal of the solvent 180 mg oil containing 45 area-% of one sofosbuvir diastereomer were isolated.

### Comparative Example 6: Synthesis of Compound of Formula (I-S) Sofosbuvir Starting from Compound (II-b′) Diethylzink Mediated

To a solution of 51 mg deoxy-fluoro-methyl uridine (0.20 mmol) in 2 mL THF 200 microL diethylzinc solution in hexane (1 M, 1.02 eq) were added. The occurring suspension was stirred for 30 min. Afterwards 79.3 mg (0.20 mmol, 1.0 eq) of (2S)-isopropyl 2-(((2-oxobenzo[d]oxazol-3(2H)-yl)(phenoxy)phosphoryl)amino)propanoate dissolved in 2 mL THF were added. After complete conversion the reaction was hydrolyzed by the addition 10 mL aqueous ammonium chloride and extracted with 20 mL ethyl acetate. The organic phase was washed with water and the solvent was removed to dryness. 110 mg of a solid containing one sofosbuvir isomer were isolated (70 area-%).

### Example 5: Synthesis of Compound of Formula (I-S) Sofosbuvir Lewis Acid Mediated and with Penta-Fluorophenol as LG

5 g deoxyfluoromethlyuridine (19.3 mmol, 1 eq) was dissolved in 200 mL THF at room temperature. To the solution 9.6 g (S)-isopropyl 2-(((S)-(perfluorophenoxy)(phenoxy) phosphoryl)amino)propanoate (21.2 mmol, 1.1 eq) prepared according J. Org. Chem 2011, 76, 8311, 4.3 g zinc bromide (19.3 mmol, 1 eq) and 2.7 mL triethyl amine (19.3 mmol, 1 eq) were added. The mixture was stirred for 20.5 h until complete conversion was observed and formation of the double phosphorylated by product was below 4 area-%. The mixture was hydrolyzed with 200 mL 1 M aqueous HCl, and THF was removed via distillation. The aqueous phase was extracted with ethyl acetate and the organic phase was washed with sodium carbonate. The organic solvent was removed to dryness, obtaining 9.7 g of a white solid containing 68 area-% of sofosbuvir (64% yield).

### CITED PRIOR ART

WO 2005/003147
WO 2008/121634
WO 2011/123668
WO 2010/135569
WO 2011/123645
WO 2011/123672
WO 2014/047117
J. Med. Chem. 2005, 48, 5504

1. A process for the preparation of a compound of formula (I) including all isomers, stereoisomers, enantiomers and diastereomers thereof 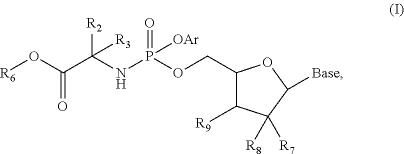
and salts thereof, the process comprising
a) providing a mixture comprising a compound of formula (II) 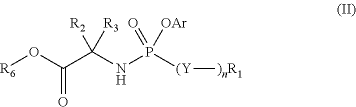
and a compound of formula (III) 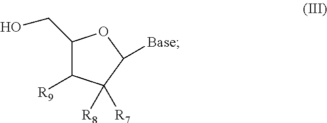
b) subjecting the mixture provided in a) to reaction conditions in the presence of one or more Lewis acids to the mixture, obtaining a mixture comprising the compound of formula (I);
wherein at each occurrence
Ar is phenyl, naphthyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
(Y—)ₙR₁ is a leaving group for nucleophilic substitution reaction, wherein n is 0 or 1 and wherein Y is O, N or S;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, NO₂, COOH, CHO, C(O)(C₁-C₆ alkyl), C(O)(aryl), COO(C₁-C₆ alkyl), COONH₂, COONH (C₁-C₆ alkyl) and CN;
R₆ is C₁-C₆ alkyl or C₃-C₁₀ cycloalkyl optionally substituted with at least one of C₁-C₆ alkyl and aryl;
Base is a purinyl residue or a pyrimidinyl residue linked to the furanose ring according to formula (III) through a carbon or nitrogen atom;
R₇ and R₈ are independently H, OH, F, Cl, Br, I, azide, nitrile, NH₂, NHR₂₆, NR₂₆R₂₄, (CO)—NH₂, (CO)—NHR₂₆, (CO)—NR₂₆R₂₄, C₁-C₆ alkyl optionally substituted with C₁-C₆ alkyl, or C₃-C₁₀ cycloalkyl optionally substituted with C₁-C₆ alkyl, wherein R₂₆ and R₂₄ are independently C₁-C₆ alkyl;
R₉ is H, OH, C₁-C₆ alkoxy, OC(O)R₂₅, or C₁-C₆ alkyl optionally substituted with C₁-C₆ alkyl or aryl, wherein R₂₅ is C₁-C₆ alkyl or aryl.

2. The process of claim 1, wherein, when n is 1,
R₁ is alkyl, aryl, or heteroaryl, each optionally substituted with one or more electron-withdrawing groups, or
R₁ is
a residue of formula (A) 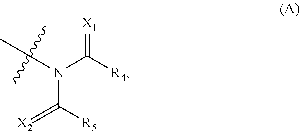
a residue of formula (B) 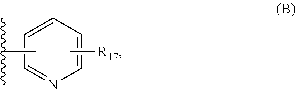
a residue of formula (C) 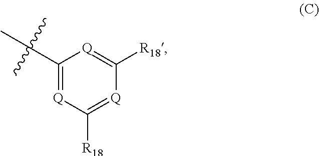
or a residue of formula (D) 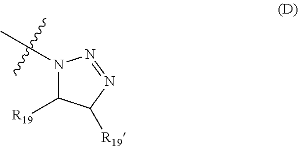
or wherein, when n is 0,
R₁ is a residue of formula (A1) 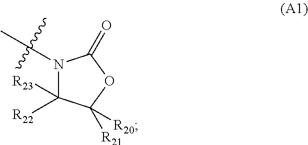
wherein at each occurrence
X₁ and X₂ are independently O or S;
R₄ and R₅ are independently H, OH, NH₂, C₁-C₆ alkyl or C₁-C₆ alkoxy, or R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, or a heterocycle comprising one or more heteroatoms independently being N, O or S;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, or a heterocycle comprising one or more heteroatoms independently being N, O or S.

3-8. (canceled)

9. The process of claim 1, wherein R₁ is a residue of formula (IIb) 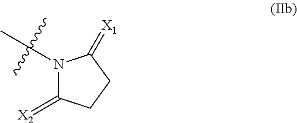
wherein X₁ is O and X₂ is O.

10-11. (canceled)

12. The process of claim 1, wherein the Lewis acid is selected from the group consisting of ZnBr₂, ZnCl₂, ZnI₂, MgBr₂, MgBr₂.OEt₂, CuCl₂, Cu(acetylacetonate)₂, and Fe(II) fumarate.

13-19. (canceled)

20. The process of claim 1, wherein the compound of formula (II) is a compound of formula (II-A)

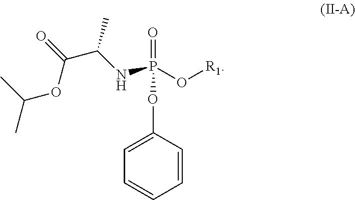

21. (canceled)

22. The process of claim 1, wherein the compound of formula (I) is the compound of formula (I-A)

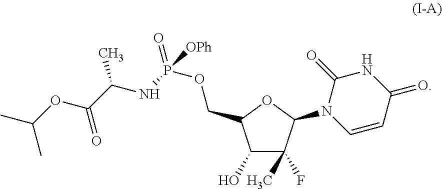

23. The process of claim 1, wherein the one or more Lewis acids according to b) comprise a twice positively charged ion, or a three times positively charged ion.

24-31. (canceled)

32. The process of claim 1, further comprising providing the mixture according to a) by a process comprising
(i) providing a mixture comprising a compound of formula (IV) 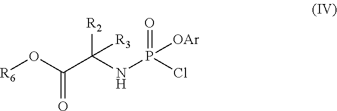
and a compound R₁(—Y)ₙH;
(ii) subjecting the mixture provided in (i) to reaction conditions, obtaining a mixture comprising the compound of formula (II).

33-59. (canceled)

60. A composition of which at least 99.90 weight-% consists of the compound of formula (II) 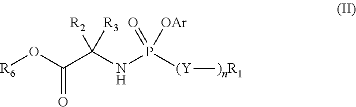
wherein
Ar is phenyl, naphthyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, COOH, CHO, C(O)(C₁-C₆ alkyl), C(O)(aryl), COO(C₁-C₆ alkyl), COONH₂, COONH(C₁-C₆ alkyl) and CN;
R₆ is C₁-C₆ alkyl or C₃-C₁₀ cycloalkyl optionally substituted with at least one of C₁-C₆ alkyl and aryl;
(Y—)ₙR₁ is a leaving group for nucleophilic substitution reaction, wherein n is 0 or 1 and wherein Y is O, N or S;
wherein, when n is 1,
R₁ is a residue of formula (A) 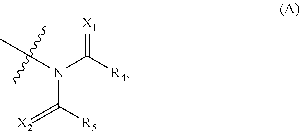
a residue of formula (B) 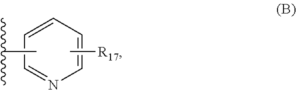
a residue of formula (C) 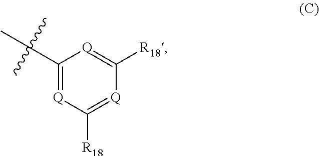
or a residue of formula (D) 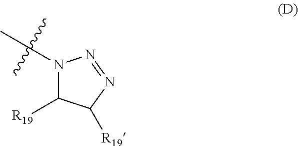
or wherein, when n is 0,
R₁ is a residue of formula (A1) 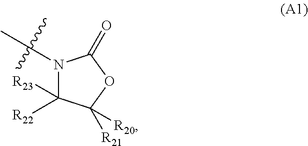
wherein at each occurrence
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, or a heterocycle comprising one or more heteroatoms independently being N, O or S;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, or a heterocycle comprising one or more heteroatoms independently being N, O or S;
and wherein
said composition has a content, based on the weight of the mixture, of less than 100 weight-ppm of an aryl-OH compound substituted with one or more electron-withdrawing groups.

61-65. (canceled)

66. The composition of claim 60, wherein R₁ is a residue of formula (IIb) 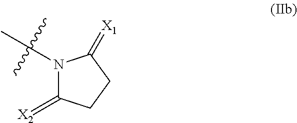
wherein X₁ is O and X₂ is O.

67-75. (canceled)

76. A compound of formula (II) 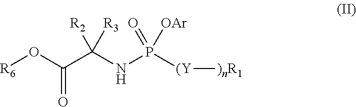
wherein
Ar is phenyl, naphthyl, quinolinyl, isoquinolinyl, quinazolinyl or quinoxalinyl, each optionally substituted with at least one of C₁-C₆ alkyl, C₁-C₆ alkoxy, C₁-C₆ cycloalkyl, aryl, halogen, COOH, CHO, C(O)(C₁-C₆ alkyl), C(O)(aryl), COO(C₁-C₆ alkyl), COONH₂, COONH(C₁-C₆ alkyl) and CN;
R₂ and R₃ are independently H or C₁-C₆ alkyl optionally substituted with at least one of OH, C₁-C₆ alkoxy, aryl, heteroaryl, C₁-C₆ alkyl, C₃-C₆ cycloalkyl, F, Cl, Br, I, COOH, CHO, C(O)(C₁-C₆ alkyl), C(O)(aryl), COO(C₁-C₆ alkyl), COONH₂, COONH C₁-C₆ alkyl) and CN;
R₆ is C₁-C₆ alkyl or C₃-C₁₀ cycloalkyl optionally substituted with at least one of C₁-C₆ alkyl and aryl;
(Y—)ₙR₁ is a leaving group for nucleophilic substitution reaction, wherein n is 0 or 1 and wherein Y is O, N or S;
wherein, when n is 1,
R₁ is alkyl, aryl, or heteroaryl, each optionally substituted with one or more electron-withdrawing groups; or
R₁ is a residue of formula (A) 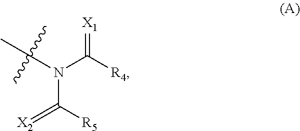
a residue of formula (B) 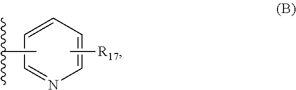
a residue of formula (C) 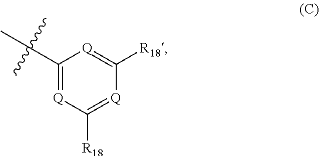
or a residue of formula (D) 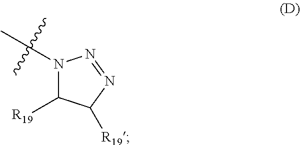
or wherein, when n is 0,
R₁ is a residue of formula (A1) 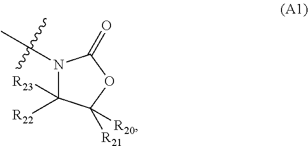
wherein at each occurrence
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, or a heterocycle comprising one or more heteroatoms independently being N, O or S;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, or a heterocycle comprising one or more heteroatoms independently being N, O or S.

77. The compound of formula (II) of claim 76, wherein (Y—)ₙR₁ is a leaving group for nucleophilic substitution reaction, wherein n is 0 or 1 and wherein Y is O, N or S;
wherein, when n is 1,
R₁ is a residue of formula (A) 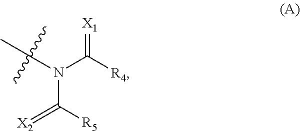
a residue of formula (B) 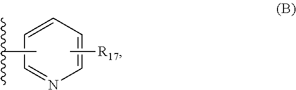
a residue of formula (C) 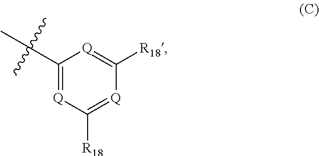
or a residue of formula (D) 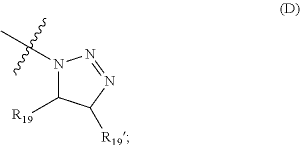
or wherein, when n is 0,
R₁ is a residue of formula (A1) 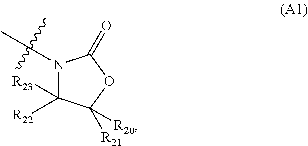
wherein at each occurrence
X₁ and X₂ are independently O or S;
R₄ and R₅ are independently H, OH, NH₂, C₁-C₆ alkyl or C₁-C₆ alkoxy, or
R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;
R₁₇ is an electron-withdrawing group;
R₁₈ and R_{18′} are independently F, Cl, Br, I, or C₁-C₆ alkoxy;
each Q is independently C or N, wherein at least one Q is N;
R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or
R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, or a heterocycle comprising one or more heteroatoms independently being N, O or S;
R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or
R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, or a heterocycle comprising one or more heteroatoms independently being N, O or S.

78-92. (canceled)

93. The process of claim 1 wherein the one or more Lewis acids according to b) comprise a Zn ion, a Mg ion, a Cu ion, or an Fe ion, or a Mn ion.
